SEQUENCE LISTING (187 sequences)

SEQ ID NO: 1 - protein, 368 aa, Trypanosoma brucei
MLRRCFTPSHLIPFLCGGCLSSRRQLVVGNSKNGTKPVASVDWTDLVGKELWSENRLAAGELLAMMDLCAKRVSEVYLSSMKPRVSCSTVGVSTTTFYSPVLHGDVVRMHGRLIYCGSSSMGIHIRFYRRSPSTITESLTGESYFTMVAIDENLRAVKTVPAVELTDENDVSYHNGYLAARNIAAEAKKISSDLQKKSITCSDVECTVNAAKPIRVPIASTRTSAHRIFLLYHLNNNRTIFGGELMRWMERHAVHCGRMFTGNRHISTVAMHNVEFHHPVFGTDWVTLEADIIYVHNTTMEVDVKLVVERDGHVVTTNRASFVLINLNEIGQKTIIPKGLDLSAATEEELLRFAEAKERYRRSRNSRR

SEQ ID NO: 2 - protein, 368 aa, Trypanosoma brucei
MLRRCFTPSHLIPFLCGGCLSSRRQLVVGNSKNGTKPVASVDWTDLVGKELWSENRLAAGELLAMMDLCAKRVSEVYLSSMKPRVSCSTVGVSTTTFYSPVLHGDVVRMHGRLIYCGSSSMGIHIRFYRRSPSTITESLTGESYFTMVAIDENLRAVKTVPAVELTDENDVSYHNGYLAARNIAAEAKKISSDLQKKSITCSDVECTVNAAKPIRVPIASTRTSAHRIFLLYHLNNNRTIFGGELMRWMERHAVHCGRMFTGNRHISTVAMHNVEFHHPVFGTDWVTLEADIIYVHNTTMEVDVKLVVERDGHVVTTNRASFVLINLNEIGQKTIIPKGLDLSAATEEELLRFAEAKERYQRSRNNRR

SEQ ID NO: 3 - protein, 353 aa, Trypanosoma brucei
MWRVFEQSAATVVGNSKNGTKPVASVDWTDLVGKELWSENRLAAGELLAMMDLCAKRVSEVYLSSMKPRVSCSTVGVSTTTFYSPVLHGDVVRMHGRLIYCGSSSMGIHIRFYRRSPSTITESLTGESYFTMVAIDENLRAVKTVPAVELTDENDVSYHNGYLAARNIAAEAKKISSDLQKKSITCSDVECTVNAAKPIRVPIASTRTSAHRIFLLYHLNNNRTIFGGELMRWMERHAVHCGRMFTGNRHISTVAMHNVEFHHPVFGTDWVTLEADIIYVHNTTMEVDVKLVVERDGHVVTTNRASFVLINLNEIGQKTIIPKGLDLSAATEEELLRFAEAKERYRRSRNNRR

SEQ ID NO: 4 - DNA, 1107 bp, Artificial Sequence
  TbACT1 codon-optimized coding sequence
atgctacgaaggtgctttaccccctcacatcttatcccattcttatgtggtggctgtctatcttctagacggcaattggtagttggaaacagtaagaatggtacgaaacctgtcgcatccgttgactggactgatctcgttgggaaggaattatggagtgaaaacaggttagcagccggtgaattgttggccatgatggatctttgtgcgaaaagagtgtccgaggtctacctcagttcgatgaaaccgagagttagctgcagcacagtaggagtttcaaccacaactttctactccccagtattgcacggtgacgttgtgagaatgcatggtagactaatctattgtggatcatccagtatggggatacatatccggttttatcgtagaagtccatcgacaattaccgagtcattgacaggcgagtcttactttactatggtggcaatcgatgaaaaccttcgagctgtcaagacggtccccgctgttgaattgacggacgagaacgacgtgagttaccacaacggatatctcgctgcacgtaatattgcagcggaggcgaagaaaatatcaagcgatcttcaaaagaaatccataacgtgctcagacgtcgagtgtacagttaacgcggcaaagccaatcagggtgccgattgcttccactcgaacatcggctcatcgtatttttttgctctaccacttgaataacaaccggaccattttcggaggtgaattgatgaggtggatggaacgacacgccgtgcactgcgggcgaatgttcaccggcaatcgtcatatatcaacagtggccatgcacaatgtagagtttcaccatccagtcttcggcacagactgggtgacgttggaggcagatatcatctatgtacataacacgactatggaggtcgatgttaagctagttgttgaaagggacggacatgttgtgaccactaacagagcttcgttcgtccttatcaaccttaatgaaattggacagaagacaattattcctaaggggttggatttgtctgcagccaccgaagaagaattattgagatttgccgaagctaaagaacgttacagacgaagccggaatagcagaagatga

SEQ ID NO: 5 - DNA, 1107 bp, Trypanosoma brucei
atgttgcgtcgctgttttaccccatcgcatttgatcccgtttttatgtggcgggtgtttgagcagtcggcggcaactcgtcgtaggcaatagtaaaaacggcacgaagcctgttgctagtgtggactggacagaccttgtgggaaaggaactgtggtcagaaaaccgactggcagcgggtgagttactggcaatgatggatttatgcgccaaacgcgtttcggaagtttatctcagctccatgaagccgcgtgtaagctgcagcacagtgggtgtaagtaccacaacattttactcccctgttctgcacggtgatgtggttcgcatgcatggacgcttaatttactgcggaagcagttccatgggaattcacatccgcttctacagacgatcaccatctaccataaccgaatctctcacgggtgaaagctatttcacgatggtggcaattgatgaaaatttgcgcgccgttaaaaccgttcccgcggttgagcttaccgatgaaaatgatgtgagttaccacaatggctacttggctgcacgcaatattgccgctgaggcaaaaaagatatcatctgatttgcagaagaaatccataacttgtagcgatgtggaatgcactgttaacgcggccaaacccattcgtgtaccgatcgcctccacgcgcacctctgcccatcgcatttttttgctttaccacctgaataataaccgaaccatctttggtggtgaattgatgcgttggatggagcgacatgccgttcattgcggtcgcatgttcacgggcaatcgtcacatctcaacggttgccatgcataatgtggagttccatcatcccgtgtttggtactgattgggtaacgttggaggcggatattatttatgttcacaacacaacaatggaggttgacgtcaaacttgtggtggaacgcgatggccatgtcgttaccacaaaccgtgcctcttttgtgttgattaatctaaacgagatcgggcagaagacgattattccgaaaggattagatttgagtgcagcgacggaggaggaactgctacgtttcgctgaggcaaaggagcggtatcagaggtcgcgcaataacaggcggtag

SEQ ID NO: 6 - DNA, 1062 bp, Trypanosoma brucei
atgtggcgggtgtttgagcagtcggcggcaactgtcgtaggcaatagtaaaaacggcacgaagcctgttgctagtgtggactggacagaccttgtgggaaaggaactgtggtcagaaaaccgactggcagcgggtgagttactggcaatgatggatttatgcgccaaacgcgtttcggaagtttatctcagctccatgaagccgcgtgtaagctgcagcacagtgggtgtaagtaccacaacattttactcccctgttctgcacggtgatgtggttcgcatgcatggacgcttaatttactgcggaagcagttccatgggaattcacatccgcttctacagacgatcaccatctaccataaccgaatctctcacgggtgaaagctatttcacgatggtggcaattgatgaaaatttgcgcgccgttaaaaccgttcccgcggttgagcttaccgatgaaaatgatgtgagttaccacaatggctacttggctgcacgcaatattgccgctgaggcaaaaaagatatcatctgatttgcagaagaaatccataacttgtagcgatgtggaatgcactgttaacgcggccaaacccattcgtgtaccgatcgcctccacgcgcacctctgcccatcgcatttttttgctttaccacctgaataataaccgaaccatctttggtggtgaattgatgcgttggatggagcgacatgccgttcattgcggtcgcatgttcacgggcaatcgtcacatctcaacggttgccatgcataatgtggagttccatcatcccgtgtttggtactgattgggtaacgttggaggcggatattatttatgttcacaacacaacaatggaggttgacgtcaaacttgtggtggaacgcgatggccatgtcgttaccacaaaccgtgcctcttttgtgttgattaatctaaacgagatcgggcagaagacgattattccgaaaggattagatttgagtgcagcgacggaggaggaactgctacgtttcgctgaggcaaaggagcggtatcggaggtcgcgcaataacaggcggtag

SEQ ID NO: 7 - protein, 403 aa, Candida viswanathii
MALPPVYIVSTARTPIGSFQGSLSSLTYSDLGAHAVKAALAKVPQIKPQDVDEIVFGGVLQANVGQAPARQVALKAGLPDSIVASTINKVCASGMKAVIIGAQNIICGTSDIVVVGGAESMSNTPYYLPSARSGARYGDAIMVDGVQKDGLLDVYEEKLMGVAAEKCAKDHGFSREDQDNFAINSYKKAGKALSEGKFKSEIAPVTIKGFRGKPDTVIENDEEIGKFNEERLKSARTVFQKENGTVTAPNASKLNDGGAALVLVSEAKLKQLGLKPLAKISGWGEAARTPFDFTIAPALAVPKAVKHAGLTVDRVDFFELNEAFSVVGLANAELVNIPLEKLNVYGGAVAMGHPLGCSGARIIVTLLSVLTQEGGRFGVAGVCNGGGGASAVVIEKIDADAKL

SEQ ID NO: 8 - protein, 403 aa, Candida viswanathii
MTLPPVYIVSTARTPIGSFQGSLSSLTYSDLGAHAVKAALAKVPQIKPQDVDEIVFGGVLQANVGQAPARQVALKAGLPDSIIASTINKVCASGMKAVIIGAQNIICGTSDIVVVGGAESMSNTPYYLPSARSGARYGDAVMVDGVQKDGLLDVYEEKLMGVAAEKCAKDHGFSREDQDNFAINSYKKAGKALSEGKFKSEIAPVTIKGFRGKPDTVIENDEEIGKFNEDRLKSARTVFQKENGTVTAPNASKLNDGGAALVLVSEAKLKQLGLKPLAKISGWGEAARTPFDFTIAPALAVPKAVKHAGLTVDRVDFFELNEAFSVVGLANAELVKIPLEKLNVYGGAVAMGHPLGCSGARIIVTLLSVLTQEGGRFGAAGVCNGGGGASAIVIEKIDSDAKL

SEQ ID NO: 9 - protein, 392 aa, Yarrowia lipolytica
MEPVYIVSTARTPIGSFLSSLSGQTYVDLGAHAVKAALAKTDIKPDQVDEIIFGNVLSAGVGQAPARQVALKAGLPDTIVATTVNKVCASGMKAIIQGAQAIMTGSADIVIAGGAESMSNVPHYVQARVANKYGNGSLVDGIQRDGLFDAYDGQAMGVAAEVCADTHSISREEQDEFAIGSYKKTQAAYAAGKFKDEIAPIELPGFRGKPGVVVSEDEEYKNLNEDKLKSARTVFKKDGTVTAPNASPINDGGAAVILASAAKVKELGLKPLLKIVSWGEAANEPVKFTTAPALAVPVALKRAGLEAKDIDFYEFNEAFSVVGIANTKLLGLDSSKVNVYGGAVAIGHPLGCSGARVIVTLNSVLHQEGGKYGCAAICNGGGGASSIIVEKC

SEQ ID NO: 10 - protein, 404 aa, Cyberlindnera jadinii
MSENSAYIVAVARTPIGSFQGSLSSQNYVDLGSHAVKAALAKVPEIKATDVEEIVFGNVVQANVGQNPARQVAIKSGMSVQTVASTVNKVCASGLKATIIAAQTIILGNADIVVAGGAESMTNAPYYVPAARSGARYGDSVLVDGLARDGLKDAYSDDAMGVCAEKCARDHGFSREDQDNFAIDSYKKAQAAHAAGKFKSEIAPVTIKGFRGKPDVVVEVDEESTKFNEQKLKTARTVFQKENGTVTAANASPINDGGAALVLVSHKKLVELNLKPIARILGWGEAAQNPEDFTTSPALAVPKALKHAGITIDDVDFFEFNEAFSVVGLANQKILGLPSEKLNVYGGAVAIGHPLGCSGARILVTLTSVLQQEGGKIGVAAICNGGGGASSMVLEKLSSEHVIL

SEQ ID NO: 11 - DNA, 1212 bp, Artificial Sequence
  CvERG10 IA codon-optimized coding sequence
atggcattgccaccagtttacattgtttcaacagcaagaacaccaattggatcatttcaaggatcattgtcatcattgacatacagtgatttgggagcacatgcagttaaggcagctttggcaaaggttccacaaattaagccacaagatgttgatgaaattgtttttggaggagttttgcaagcaaacgttggacaagcaccagcaagacaagttgcattgaaggcaggattgccagattcaattgttgcatcaacaattaacaaggtttgtgcatcaggaatgaaggcagttattattggagcacaaaacattatttgtggaacatcagatattgttgttgttggaggagcagaatcaatgtcaaacacaccatactacttgccatcagcacgatcaggagcaagatacggagatgcaattatggttgatggagttcaaaaggatggattgttggatgtttacgaagaaaagttgatgggagttgcagcagaaaagtgtgcaaaggatcatggattttcacgagaagatcaagataactttgcaattaactcatacaagaaggcaggaaaggcattgtcagaaggaaagttcaagtcagaaattgcaccagttacaattaagggatttcgaggaaagccagatacagttattgagaacgatgaagaaattggaaagtttaacgaagaaagattgaagtcagcaagaacagtttttcaaaaggaaaacggaacagttacagcaccaaacgcatcaaagttgaacgatggaggagcagcattggttttggtttcagaagcaaagttgaagcaattgggattgaagccattggcaaagatttcaggatggggagaagcagcaagaaccccatttgattttacaattgcaccagcattggcagttccaaaggcagttaagcatgcaggattgacagttgatagagttgatttttttgaattgaacgaagcattttcagttgttggattggcaaacgcagaattggttaacattccattggaaaagttgaacgtttacggaggagcagttgcaatgggacatccattgggatgttcaggagcacgaattattgttacattgttgtcagttttgacacaagaaggaggaagatttggagttgcaggagtttgtaacggaggaggaggagcatcagcagttgttattgaaaagattgatgcagatgcaaagttgtga

SEQ ID NO: 12 - DNA, 1212 bp, Candida viswanathii
atgaccctcccaccagtctacatcgtctccacagcaagaactccaattggttccttccaaggttcgttatcttccttgacctactccgatttgggtgcccacgccgttaaggcagctttagccaaggtcccacagatcaagccacaggacgtcgacgagatcgtctttggtggtgtcttgcaagccaacgttggtcaggcaccagccagacaagtcgccttgaaggcaggcttgccagactctatcattgcctccaccattaacaaagtctgtgcctccggtatgaaggccgtcattatcggtgcccaaaacatcatctgtggaaccagtgacatcgtcgttgttggtggtgccgaatccatgtctaacactccatactacttgccaagcgccagaagcggggccagatacggtgacgccgtcatggttgatggtgtccagaaggatggtttgttggatgtctacgaagaaaaattgatgggtgttgctgctgaaaagtgtgccaaggaccacggattcagcagagaagaccaggacaactttgccatcaactcttacaagaaggctggcaaggctttgagtgaaggtaagttcaaatcagaaatcgccccagtcaccattaaaggattcagaggcaagccagatactgttattgaaaatgatgaagagattggcaaattcaacgaagacagactcaagtctgccagaactgtcttccaaaaggaaaacggtactgttactgctccaaacgcttctaaattgaacgatggtggtgctgccttggtcttggtttctgaagccaagttgaagcaattgggcttgaagccattggccaagatctctggttggggtgaagctgccagaactccattcgatttcaccattgccccagctttggctgttccaaaggctgtcaagcacgctggtttgaccgttgacagagtcgacttctttgaattgaacgaagccttctccgttgttggtttggccaacgctgaattggtcaagatcccattggaaaaattgaacgtctacggtggtgctgtcgccatgggtcacccattgggttgcagtggtgctagaattattgtcaccttgttgtccgtcttgacccaagaaggcggtagatttggtgctgctggtgtctgtaacggtggtggtggtgcctctgccatcgtcattgaaaagattgactccgatgccaagttgtaa

SEQ ID NO: 13 - DNA, 1179 bp, Yarrowia lipolytica
atggagcccgtctacattgtttctactgctcgaacccccattggttcttttctgagctcgctgagcggccagacctacgtggatctgggagcccatgccgtgaaggccgcactcgccaagaccgacatcaagcccgaccaggtcgacgagatcatcttcggtaacgttctctccgccggcgtcggacaggcccctgctcgacaggttgccctcaaggctggtctccctgacaccattgtcgctaccaccgtcaacaaggtctgtgcctccggtatgaaggccatcatccagggtgcccaggccatcatgaccggatctgccgacattgtcattgctggcggtgccgagtccatgtccaacgtgccccactacgtgcaggcccgtgtcgctaacaagtacggcaacggctctctggtcgacggtatccagcgagacggtctgtttgacgcctacgatggccaggccatgggtgtggccgctgaggtctgtgctgacacccactccatctcccgagaggagcaggacgagtttgccattggctcttacaagaagacccaggctgcctatgccgccggcaagtttaaggacgagattgcccccattgagctgcccggcttccgaggcaagcctggtgtcgttgtctccgaggacgaggagtacaagaacctcaacgaggacaagctcaagtctgcccgaactgtttttaagaaggacggtaccgtgactgcccccaacgcctcccctatcaacgacggtggagctgccgtcattctggcctctgctgccaaggtcaaggagcttggtcttaagcccctgctcaagattgtctcttggggcgaggctgccaacgagcccgtcaagttcaccactgctcccgctctggccgtccccgttgccctcaagcgagctggtctcgaggccaaggacattgacttttacgagttcaacgaggccttctctgttgttggtatcgccaacaccaagctgcttggtctggactccagcaaggtcaacgtctacggaggtgctgttgccattggtcaccctctgggctgctctggtgcccgagtcattgtcactctcaactctgttctgcaccaggaaggcggcaagtacggatgtgctgccatctgcaacggtggtggtggcgcttcctctatcattgttgagaagtgttag

SEQ ID NO: 14 - DNA, 1215 bp, Artificial Sequence
  CjERG10 codon-optimized coding sequence
atgtccgagaattctgcttacattgttgctgttgctagaactcccattggttcttttcaaggttcgttgtcctcccaaaactatgtcgatcttggctcccacgcggtcaaggctgccttagccaaagttccagagatcaaggccactgacgtagaagagattgttttcgggaatgtcgtacaagccaacgtcgggcagaacccagcaagacaagtagctattaaaagtggtatgtcagtccagacggtggcatcaacggtgaataaggtgtgtgcttcaggattgaaagcaacaattattgctgctcagaccataatattgggtaatgctgacatagttgttgcgggaggagccgaaagcatgaccaatgcaccctattacgtgcctgcggcaaggagtggggcaaggtacggggactcagtgctagtcgatggtttggcgcgagatggattgaaagatgcgtattctgatgacgcaatgggtgtttgcgccgaaaaatgcgccagagatcacggattttcgcgggaagaccaagacaactttgcaatcgatagttataagaaagcccaggcggcccacgcagccggcaagttcaaaagtgaaatcgcacccgttacaatcaaaggcttccggggtaagccagacgttgtagttgaggtcgatgaggagtccacgaagttcaacgaacaaaagttgaagactgctcgtacagtctttcaaaaggaaaacggcactgtgacagctgccaatgcctcaccaattaatgatggcggagcggcactcgtccttgtctcacataaaaaattggttgaactcaacctaaagccgattgcgcgaatattgggttggggagaagctgcacagaacccggaggacttcacgaccagccctgcacttgcggtgccaaaggccctcaagcatgccggaatcacaatcgatgacgtagacttttttgagttcaacgaagcttttagtgtggttgggttggctaaccaaaagattttgggacttccatcagagaagttgaacgtgtacggcggggctgttgcaatcgggcatccattaggttgttcgggcgccagaatcctagtgacattaacctcggttcttcagcaagaaggcggaaaaatcggagtggcagcaatatgtaatggtggaggtggtgcaagctctatggtactagaaaagctcagcagcgaacatgttatcctttga

SEQ ID NO: 15 - protein, 218 aa, Clostridium
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 16 - protein, 224 aa, Artificial Sequence
  CbCTF1-P
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPAGRRAKL

SEQ ID NO: 17 - protein, 221 aa, Clostridium
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 18 - protein, 227 aa, Artificial Sequence
  CbCTF2-P
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAVGRRAKL

SEQ ID NO: 19 - protein, 217 aa, Clostridium saccharobutylicum
MNKIVKLEDLKHVFKDNMTIMVGGFLDCGTPENIIDMLVDLNVKNLTIISNDTAFPDKGIGKLIVNGQVSKVIASHIGTNPETGKKMNNGEIEVELSPQGTLIERIRAAGSGLGGVLTPTGLGTIVEEGKRKVTIGGKEYLLELPLCADVSIVKGSVVDEFGNTCYRATTKNFNPYMAMAGKIVIVEAENLVKCEQLKRENIMTPGVLVNYIVKEAA

SEQ ID NO: 20 - protein, 221 aa, Clostridium saccharobutylicum
MIQDKTIAKKIIAKRVAKELKEGQLVNLGIGLPSLVANYVPKEMNITFQSENGIVGMGKMAERDKEDEDIINAGGQCATLLPHGAFLDSSMSFSLIRGGHVDVAVLGALEVDEKGNLANWIVPNKIVPGMGGAMDLAIGAKRIIVAMQHTGKGKPKILKKCNLPLTAKSQVDLIVTELGVIKVISDGLLLKEINKDTTIDEIKSLTEADLIIPPDIKTMDV

SEQ ID NO: 21 - protein, 217 aa, Clostridium algidicarnis
MNKIVELKDVKHLFKDGMTIMIGGFLGCGSAHDIIDYIVDLNIKDLTVITNDTCYPGTGVGKLLLNGQIKKLIASYIGGNNDTGRLMNEGKLEAELIPQGTLAEKIRAGGSGLGAVITATGVGTIVEDGKDKLTIDGKEYLIELPLRADMSILRGTIVDESGNIFYRGTTKNFNPVMAMASDIVIVEAEKVVKLGNLDAENVHTPSVLVDYVIKELA

SEQ ID NO: 22 - protein, 222 aa, Clostridium algidicarnis
MIQDALIAKEIIAKRVAKEMKNGQLVNLGVGLPTMVANYIPEDVHVLLQSENGMIGMGESPESGFEDKDIVNAGGQCVTILPKGCFFDSSFSFALIRGGHVDVTVLGALEVDEKGNLANWIIPGKKVPGMGGAMDLVTGAKKVIVAMQHTGKGNPKILKECRLPLTAKSVVNMIVTELCVLEVTEDGLILTEIQKDTTLDEIRSFTEADFTVAKYLKVMDEN

SEQ ID NO: 23 - protein, 217 aa, Clostridium thermoalcaliphilum
MNKVVNIDEIKHLFKDGMTIMVGGFLGCGTPEKLIDVLIDLNIKDITLIANDTSFVDKGVGRLIVNNQIKKAIVSHIGTNPQTGKLMNEGKLKVELCPQGTLIERIRAGGYGLGGILTPTGIGTVVEEGKQKIVIDEKEYLLELPLRADIALIKGNTIDEFGNVSYKGTTRNFNPIIAMAADKVIAEAEKIVKTGEIDKELIGTPGVVVDYVIKGGE

SEQ ID NO: 24 - protein, 222 aa, Clostridium thermoalcaliphilum
MIIDETVGKEIIAKRVAKELKDGQLVNLGIGLPTLVANYIPKDINVTFQSENGMVGMGSLAQRGEENINITNAGGQYVNVLPQGSFFDTCMSFGLIRGGHVDVTVLGALEVDQSGNLANWIIPGKLVPGMGGAMDLVTGAKKVIVSMLHTSKGKPKIFKKCNLPLTAREKVNLIITELGVMEVTNKGLVLREINKDVTVDQIKSLTEADLIISDDLKIMDIA

SEQ ID NO: 25 - protein, 221 aa, Escherichia coli
MKNKKILLADVIPYFRDGMTIMFGGFMGVGTPPLLIEALLESGAKDLTIIGNDTAFVDTGVGPLISRGRVKKVIASHIGTNPETGRRMIAGEMQVELVPQGTLVERIRCGGAGLGGFLTPTGVGTVVEEGKQKIVQDGVEYLLELPLRADLALIHAYTADSVGNLTYQLTARNFNPIIALAADLVIAQTDQMVDVGCIQPDQVVTPGALIDYVVVSPEVCS

SEQ ID NO: 26 - protein, 218 aa, Escherichia coli
MDKNAMRERIARRVAKELHKGDLVNLGIGLPTQVANYVADDLGVIFQSENGLVGLGPTPDSDSIDADLTNAGGQPVTAVPGAAFFDSALSFAIIRGGHVDVTVLGALEVDQEGNLANWIIPGKMVPGMGGAMDLVNGARRVIIAMEHCDKRGQSKILPRCQLPLTAVGQVNLIVTEKAVIEVNNGLVLREIAADTSVEEVVAQTAAPLRISETLIQFS

SEQ ID NO: 27 - DNA, 657 bp, Artificial Sequence
  CbCTF1 codon-optimized coding sequence
atgaactcaaagattattagatttgaaaacttgagatccttctttaaggatggaatgacaattatgattggaggatttttgaactgtggaacaccaacaaagttgattgattttttggttaacttgaacatcaagaacttgacaattatctcaaacgatacatgttacccaaacacaggaattggaaagttgatttcaaacaaccaagttaagaagttgattgcatcatacattggatcaaacccagatacaggcaagaagttgtttaacaacgaattggaagttgaattgtcaccacaaggaacattggttgaaagaattagagcaggaggatcaggattgggaggagttttgacaaagacaggattgggaacattgattgaaaagggaaagaagaagattagtattaacggaacagaatacttgttggaattgccattgacagcagatgttgcattgattaagggatcaattgttgatgaagcaggaaacacattttacaagggaacaacaaagaactttaacccatacatggcaatggcagcaaagacagttattgttgaagcagaaaacttggtttcatgtgaaaagttggaaaaggaaaaggcaatgacaccaggagttttgattaactacattgttaaggaaccagcatga

SEQ ID NO: 28 - DNA, 675 bp, Artificial Sequence
  CbCTF1-P codon-optimized coding sequence
atgaactcaaagattattagatttgaaaacttgagatcattcttcaaggatggaatgacaattatgattggaggatttttgaactgtggaacaccaacaaagttgattgatttcttggttaacttgaacattaagaacttgacaattatttccaacgatacatgttacccaaacacaggaattggaaagttgatttcaaacaaccaagttaagaagttgattgcatcatacattggatcaaacccagatacaggaaagaagttgtttaacaacgaattggaagttgaattgtcaccacaaggaacattggttgaaagaattagagcaggaggatcaggattgggaggagttttgacaaagacaggattgggaacattgattgaaaagggaaagaagaagatttcaattaacggaacagaatacttgttggaattgccattgacagcagatgttgcattgattaagggatcaattgttgatgaagcaggaaacacattttacaagggaacaacaaagaactttaacccatacatggcaatggcagcaaagacagttattgttgaagcagaaaacttggtttcatgtgaaaagttggaaaaggaaaaggcaatgacaccaggcgttttgattaactacattgttaaggaaccagcaggtagacgagcaaagctttga

SEQ ID NO: 29 - DNA, 666 bp, Artificial Sequence
  CbCTF2 codon-optimized coding sequence
atgattaacgataagaacttggcaaaggaaattattgcaaagagagttgcaagagaattgaagaacggacaattggttaacttgggagttggattgccaacaatggttgcagactacattccaaagaactttaagattacatttcaatcagaaaacggaattgttggaatgggagcatcaccaaagattaacgaagcagataaggatgttgttaacgcaggaggagattacacaacagttttgccagatggaacattttttgattcatcagtttcattttcattgattagaggaggacatgttgatgttacagttttgggagcattgcaagttgatgaaaagggaaacattgcaaactggattgttccaggaaagatgttgtcaggaatgggaggagcaatggatttggttaacggagcaaagaaggttattattgcaatgcgacacacaaacaagggacaaccaaagattttgaagaagtgtacattgccattgacagcaaagtcacaagcaaacttgattgttacagaattgggagtgatcgaagttattaacgatggattgttgttgacagaaattaacaagaacacaacaattgatgaaattagatcattgacagcagcagatttgttgatttcaaacgaattgaggccaatggcagtttga

SEQ ID NO: 30 - DNA, 684 bp, Artificial Sequence
  CbCTF2-P codon-optimized coding sequence
atgattaacgataagaacttggcaaaggaaattattgcaaagagagttgcaagagaattgaagaacggacaattggttaacttgggagttggattgccaacaatggttgcagattacatcccaaagaactttaagattacatttcaatcagaaaacggaattgttggaatgggagcatcaccaaagattaacgaagcagataaggatgttgttaacgcaggaggagattacacaacagttttgccagatggaacattttttgattcatcagtttcattttcattgattagaggaggacatgttgatgttacagttttgggagcattgcaagttgatgaaaagggaaacattgcaaactggattgttccaggaaagatgttgtcaggaatgggaggagcaatggatttggttaacggagcaaagaaggttattattgcaatgagacataccaacaagggacaaccaaagattttgaagaagtgtacattgccattgacagcaaagtcacaagcaaacttgattgttacagaattgggagttattgaagtgatcaacgatggattgttgttgaccgaaattaacaagaacacaacaattgatgaaattagatcattgacagcagcagatttgttgatttcaaacgaattgaggccaatggcagttggtagacgagcaaagctttga

SEQ ID NO: 31 - DNA, 654 bp, Artificial Sequence
  CsCTF1 codon-optimized coding sequence
atgaacaaaatcgtaaagttggaagacctcaagcatgtgttcaaagataatatgaccattatggtgggagggtttctagactgtggaactccggaaaacatcattgatatgcttgtcgatcttaacgttaagaatcttacaatcatatcgaacgacacggcatttcctgacaaaggaattggtaaattgatcgttaacggacaggtgtcgaaagttatcgcatcacatattggcaccaatccggaaacaggaaagaaaatgaataacggtgagatcgaagtagaattgtcgccacaaggaaccctcattgaacgaatcagagctgctggatcaggattgggcggagtcctaactccgactgggctaggaaccatcgttgaggaaggaaagcgtaaagtcaccattggcggaaaagaatacttattggaactaccgttatgtgctgatgtcagtatcgtgaaaggaagtgtggttgacgagtttggaaacacctgctatagagcaacaaccaaaaattttaatccctacatggccatggctggcaaaattgtgattgtggaggctgagaatcttgtaaaatgtgagcagctcaagagagagaacataatgacacccggagttttggtaaactacatcgtgaaagaggccgcctga

SEQ ID NO: 32 - DNA, 666 bp, Artificial Sequence
  CsCTF2 codon-optimized coding sequence
atgattcaggacaaaactatcgccaagaaaattattgcaaagagagtcgcaaaggaattgaaggaagggcaattggttaaccttggcattggtcttccaagcttggttgctaactacgttcccaaggaaatgaatatcacttttcaatccgagaacggaatcgttggaatgggtaagatggccgaacgtgacaaagaagacgaggacatcataaatgcgggtggtcaatgtgccacattgcttcctcacggtgcgtttttggattcttcaatgagcttttcattaatacgtggcggacacgttgacgttgcggttcttggagccctagaagtggatgaaaaaggaaacctagcaaattggattgttccaaacaagattgtgcctggaatggggggcgccatggacctagctatcggtgcaaaaagaataattgtagccatgcaacacacaggtaaaggtaaaccaaagattttaaagaaatgtaacctaccccttacggccaagagtcaagtagatttgatagtcacagagctaggtgttataaaagtgatttcggacgggttattgcttaaggagataaataaggacacaactatcgacgagatcaaatccttaaccgaggcagatttgataataccaccagatatcaagacaatggacgtttga

SEQ ID NO: 33 - DNA, 654 bp, Artificial Sequence
  CaCTF1 codon-optimized coding sequence
atgaataaaatagtcgagcttaaggatgttaagcatttattcaaagatggaatgaccattatgataggcggctttctaggatgtggttccgcccacgacattatagactatatcgttgacctcaatattaaggacctcactgtgatcactaatgacacgtgttacccaggtacaggtgtcggtaaactcctattgaacgggcagatcaagaaacttatagcaagctatatcggtggaaataatgacacagggcgtttaatgaacgaggggaagttagaggctgaattgatcccacaaggcacgctcgcagaaaaaatcagagctggaggaagtggactaggggcagtcattaccgcaaccggagttggtactattgtagaagatggcaaagacaagttgacaatcgacggcaaggagtatttgatcgaattgccattgagagcggatatgtcaatattgagaggaacgatcgtggacgagtccggtaacattttctacagaggtaccacgaagaatttcaacccagtgatggccatggctagcgatattgttattgtggaggcagagaaggtcgtaaagttggggaacttggatgcggagaatgttcatactccatctgttttggttgactatgttataaaggaattagcttga

SEQ ID NO: 34 - DNA, 669 bp, Artificial Sequence
  CaCTF2 codon-optimized coding sequence
atgatccaagatgcattgattgctaaggaaatcatagccaaacgagtagctaaagagatgaagaatgggcaattggtaaaccttggtgtcgggctacccaccatggttgctaattacattccagaagatgtccacgttttgctccagagcgaaaacggcatgattggtatgggagaaagtccagagtcaggctttgaagataaagatattgtgaatgcgggaggccagtgcgtcaccatcttgccaaagggatgtttttttgattcctcatttagtttcgccctcattagaggagggcacgtcgatgttactgttttaggggctttagaagttgatgagaagggtaatttagcgaactggatcatacctggaaagaaggttccaggtatgggtggagcaatggatctcgttacgggtgccaagaaagttattgttgctatgcaacataccggaaaggggaacccgaaaattttgaaagaatgtagacttccgttaaccgcaaagagtgtggttaacatgatcgttacagagctatgcgtgttggaggtgaccgaagatgggctcatattaacagagattcaaaaagataccacactcgatgaaatcagaagttttactgaagcagacttcacggtcgcaaagtacctcaaggtgatggatgagaattga

SEQ ID NO: 35 - DNA, 654 bp, Artificial Sequence
  CtCTF1 codon-optimized coding sequence
atgaacaaggtcgttaatatagatgagataaagcatttgtttaaggatggtatgaccattatggttggggggttcctcggatgtggaacaccagagaaactaatcgacgtcttgattgacttaaacatcaaggatatcactctaatagccaacgacaccagcttcgtagataagggtgtcggccgattaatcgtcaataatcaaatcaaaaaagccattgtcagtcatatcggaactaacccacaaaccgggaaattgatgaacgaggggaagttaaaggtcgagttgtgtccacaaggtacgcttatagagcgtatccgagccggcggatacgggctcggaggtatcctaactccaacaggtattggaacggttgttgaagagggtaaacaaaaaattgtcatcgatgagaaggagtatttacttgaactccctttaagagcggacattgctttgatcaagggcaacactattgacgagtttggaaatgtgtcctacaagggtacaacaagaaattttaaccctatcatcgcgatggcagcagacaaagttatagcagaagccgaaaagattgtgaaaacaggtgagattgataaagagctaataggtacacccggagtcgtcgtcgattacgttatcaaaggcggagagtga

SEQ ID NO: 36 - DNA, 669 bp, Artificial Sequence
  CtCTF2 codon-optimized coding sequence
atgataattgatgagacagttggtaaggagatcatcgccaaacgagtggcaaaggagcttaaggacggtcaattggtcaacctaggcatcgggttacctacacttgtcgccaactatatacctaaagatatcaatgtcacgtttcaaagcgagaacggtatggttggcatggggtccttggcacaaagaggtgaagaaaacataaatatcaccaacgctggcgggcagtatgtgaacgtgcttccacagggaagtttctttgatacatgcatgagttttggactaatccgagggggtcacgtggatgtcaccgtgcttggcgcccttgaagttgaccagagcggtaatcttgcaaactggatcatcccaggcaaacttgttccaggtatggggggggcgatggacctagtgacaggtgcgaagaaggtaattgtcagcatgcttcatacgtccaagggaaaaccgaaaattttcaagaagtgcaatttgcccttgactgcgcgtgaaaaggtgaacttgatcattacagaattgggtgtgatggaagtgacaaacaagggccttgtccttcgagagattaataaggacgttaccgtggatcaaataaagtcactcaccgaggctgatttgatcatttcggacgacttaaagatcatggatatagcgtga

SEQ ID NO: 37 - DNA, 666 bp, Artificial Sequence
  EcCTF1 codon-optimized coding sequence
atgaagaacaagaaaattttgctagcagatgtaataccatacttccgagatggtatgaccataatgtttggcggttttatgggggttggtacgccacccctacttattgaagcccttttggagtccggtgccaaggatttaacgataataggcaacgacactgcattcgttgacacgggtgtggggccacttatttcaagggggcgagttaagaaagtaattgcatctcacatagggacaaatccagagactggaaggagaatgattgccggtgaaatgcaagtagagttagtgccacagggcacgttggttgagcgtatccgatgtggaggcgcagggcttggtggatttttgacgccaacaggcgtgggcaccgttgtagaggaagggaaacagaagatcgtacaagacggcgttgaatatttgttggagcttccattaagagctgacttggcccttattcatgcgtataccgccgattcagtaggtaacctcacttatcaactaacggcgaggaattttaatccgatcattgcgttggcggcggacttagtcatcgcgcagacggatcaaatggttgacgtaggctgtattcagcccgaccaagtagttacgccgggcgcattgatagactatgttgttgtgagcccggaagtttgttcttga

SEQ ID NO: 38 - DNA, 657 bp, Artificial Sequence
  EcCTF2 codon-optimized coding sequence
atggataagaacgctatgcgagaaagaattgctcgaagggttgcgaaggagttacacaaaggtgatctcgtgaatctcggaataggcctaccaacacaagtagcaaactatgttgcagatgatctcggagtgatctttcagagcgaaaatggacttgtcgggttgggccctacgccagattcagattcgatcgatgccgacctcacaaatgcgggtggacaaccggtgactgcagtgcctggggctgcgttcttcgacagtgcattgagcttcgcaataattcgaggtgggcacgtggacgtaaccgtacttggagcattggaagtggatcaggaaggcaaccttgctaattggataattccaggaaagatggttccgggaatgggaggggcaatggatctcgtcaacggcgcaagacgtgtgatcattgcaatggaacactgtgacaagaggggtcaatccaaaatattaccaagatgtcaactccctttaactgccgttggtcaggtgaacttgatagtcacagaaaaggcggtaattgaagtgaataacgggctagttctaagagagattgccgctgacacaagtgttgaggaagtggtagcccagaccgctgcaccgctacgaatttccgaaacgttgattcagttttcatga

SEQ ID NO: 39 - protein, 136 aa, Haemophilus influenzae
MLDNGFSFPVRVYYEDTDAGGVVYHARYLHFFERARTEYLRTLNFTQQTLLEEQQLAFVVKTLAIDYCVAAKLDDLLMVETEVSEVKGATILFEQRLMRNTLMLSKATVKVACVDLGKMKPVAFPKEVKAAFHHLK

SEQ ID NO: 40 - protein, 136 aa, Haemophilus haemolyticus
MSEKTFSLPVRVYYEDTDAGGVVYHARYLHFFERARTEYLRALNFTQQTLLQEQQLAFVVKSLAIDYCVAAKLDDLLTVETDVIEIKGATILFEQRLMRDTVMLSKATVKVASVHLGNMKPVALPKEVKAAFYNLK

SEQ ID NO: 41 - protein, 135 aa, Haemophilus parainfluenzae
MLNNQCFTFPVRVYYEDTDAGGVVYHARYLHFFERARTEYLRTLNFSQQSLLQEQQVAFVVKTMSIDYCIPAKLDDYLIVETEITAVKGATILFEQRLVRETVMLSKATVKVACVDLGKMKPVAIPEELKAAFLK

SEQ ID NO: 42 - protein, 140 aa, Rodentibacter trehalosifermentans
MDSKIFRFPVRVYYEDTDAGGVVYHARYLHFFERARTEYLRKQNFSQQVLMQEQQHAFVVKSMVIDYCVPAKLDDYLIVETELTAIKGATLLFEQRLMREAIMLSKATVKVAYVDLGKMKPAAIPREIKSAFSNDITFSE

SEQ ID NO: 43 - protein, 136 aa, Rodentibacter myodis
MDSKIFRFPIRVYYEDTDAGGVVYHARYLHFFERARTEYLRKQDFSQQKLLQEQQAAFVVKAMTIDYCVPAKLDDYLIVETEVTAIKGATILFEQRLLRETVILSKATVKVAYVDLGKMKPTAIPAEIKAAFSNNI

SEQ ID NO: 44 - protein, 140 aa, Rodentibacter genomospecies 2
MVSKVFRFPVRVYYEDTDAGGVVYHARYLHFFERARTEYLRKQNFSQQILLQEQQAAFVVKSMAIDYCIPAKLDDYLIVETEVTAIKGATILFEQRLMRETVILSKATVKVAYVDLGKMKPAAIPTEIKAAFSNNITFSE

SEQ ID NO: 45 - protein, 246 aa, Bacillus
MGQLFKSFDASEKTQLICFPFAGGYSASFRPLHAFLQGECEMLAAEPPGHGTNQTSAIEDLEELTDLYKQELNLRPDRPFVLFGHSMGGMITFRLAQKLEREGIFPQAVIISAIQPPHIQRKKVSHLPDDQFLDHIIQLGGMPAELVENKEVMSFFLPSFRSDYRALEQFELYDLAQIQSPVHVFNGLDDKKCIRDAEGWKKWAKDITFHQFDGGHMFLLSQTEEVAERIFAILNQHPIIQPGSRS

SEQ ID NO: 46 - protein, 244 aa, Bacillus atrophaeus
MSQLFRSFDTSEKTQLICFPFAGGYSASFRPLHAYLQGECEMMAAEPPGHGTNQTSAIEDLEELVRLYKQELNLRPDRPFVLFGHSMGGMITFRLAQKLEREGIFPQAVIISAIQPPNVKRKKVSHLEDEAFLEHIMELGGMPAELVENKEVMSFFLPSFRSDYRALERFEPRDLGQLKSPVHIFNGRKDEKCMRDAHGWDRWAQNPVYHEFDGGHMFLLSETEKVAEEIFSILNHCKPAVSHK

SEQ ID NO: 47 - protein, 242 aa, Bacillus halotolerans
MSQLFKSFDASEKTQLICFPFAGGYSASFRPLHAFLQGECEMLAAEPPGHGTNQTSAIEDLEQLTDLYKQELHLHPDRPFVLFGHSMGGMITFRLAQKLEREGIFPQAVIISAIQPPHIQRKKVSHLPDDQFLDHIIKLGGMPAELVENKEVMSFFLPSFRSDYRALEQFELNDMTPIQSPVHVFNGLNDEKCIRDAEGWKKWAKHIMYHEFDGGHMFLLSNTEEVAERIFAILNQHPISSS

SEQ ID NO: 48 - protein, 650 aa, Sinorhizobium meliloti
MQAERPLWVPDREIVERSPMAEFIDWCGERFGRSFADYDAFHDWSVSERGAFWTAVWEHCKVIGESGEKALVDGDRMLDARFFPEARLNFAENLLRKTGSGDALIFRGEDKVSYRLTWDELRALVSRLQQALRAQGIGAGDRVAAMMPNMPETIALMLATASVGAIWSSCSPDFGEQGVLDRFGQIAPKLFIVCDGYWYNGKRQDVDSKVRAVAKSLGAPTVIVPYAGDSAALAPTVEGGVTLADFIAGFQAGPLVFERLPFGHPLYILFSSGTTGVPKCIVHSAGGTLLQHLKEHRFHCGLRDGERLFYFTTCGWMMWNWLASGLAVGATLCLYDGSPFCPDGNVLFDYAAAERFAVFGTSAKYIDAVRKGGFTPARTHDLSSLRLMTSTGSPLSPEGFSFVYEGIKPDVQLASISGGTDIVSCFVLGNPLKPVWRGEIQGPGLGLAVDVWNDEGKPVRGEKGELVCTRAFPSMPVMFWNDPDGAKYRAAYFDRFDNVWCHGDFAEWTPHGGIVIHGRSDATLNPGGVRIGTAEIYNQVEQMDEVAEALCIGQDWEDDVRVVLFVRLARGVELTEALTREIKNRIRSGASPRHVPAKIIAVADIPRTKSGKIVELAVRDVVHGRPVKNKEALANPEALDLFAGLEELKS

SEQ ID NO: 49 - protein, 650 aa, Ensifer
MQADRPLWVPDSAILERSPMAQFMGWCGQRFGRTFADYDAFHDWSVAHRGDFWTGLWEHCDVIGERGERALINGERMLDARFFPDAKLNFAENLLRKSGGGDALIFRGEDKVSYRLSWDELRALVSRLQQALKGQGVGVGDRVAAMMPNMPETIALMLATASLGAIWSSCSPDFGEQGVLDRFGQIGPKLFIACDGYWYNGKRQDVDAKVRAVAKALGVPTVIVPYAGDSAALAAVIEGGATLPDFIAGFDAKPLAFERLPFGQPLYILFSSGTTGVPKCIVHSAGGTLLQHLKEHRFHCGLKEGERLFYFSTCGWMMWNWLASGLAAGATLCLYDGSPFCPDGNVLFDYAAAERFAVFGTSAKYIDAVRKAGLTPAKTHDLASLRLITSTGSPLSPEGFSFVYEGIKPDVQLASISGGTDIVSCFVLGNPLKPVWRGEIQGPGLGLAVDVWNDEGQPVRGEKGELVCTKAFPSMPVMFWNDPDGAKYRAAYFERFDNVWCHGDFAEWTAHGGIIIHGRSDATLNPGGVRIGTAEIYNQVEQMPEVLEALCIGQDWEDDVRVILFVRLAPGVELTEELTKAIKNRIRIGASPRHVPAKVIAVADIPRTKSGKIVELAVRDVVHGRPVKNKEALANPEALDLFAELDALKS

SEQ ID NO: 50 - protein, 650 aa, Sinorhizobium fredii
MQTDRPLWVPDAETLARSPMAEFMAWCEERFGRRFSDYDAFHQWSVTERGDFWTAVWEHCGVIGERGDQALINGERMLEARFFPEAKLNFAENLLRGSGDGDALIFRGEDKVRYRLSWDELRSLVSRLQQALKAQGIGVGDRVAAMMPNLPETIALMLATASVGAIWSSCSPDFGEQGVLDRFGQIGPKLFVVCDGYWYNGKRQNVDAKVRAVAKTLGVPTLVVPYAGDSPGLAAAIDGGVTLPDFIAGFEAKPLEFERLPFGHPLYILFSSGTTGVPKCIVHSAGGTLLQHLKEHRFHCGLKEGERLFYFTTCGWMMWNWLASGLAVGATLCLYDGSPFYPDGNVLFDFAAAERFAVFGTSAKYIDAVRKGGLTPATTHDLPALRLLTSTGSPLSPEGFSFVYEGIKSDVQLASISGGTDIVSCFVLGNPLKPVWRGEIQGPGLGLAVDVWNDEGKPVRGEKGELVCTKAFPSMPVMFWNDPEGAKYRAAYFERFDNIWCHGDFAEWTPHDGIVIHGRSDATLNPGGVRIGTAEIYNQVEQMPEVAEALCIGQDWQDDVRVILFVRLAPGVALTEELSQEIKTRIRTGASPRHVPAKIIPVSDIPRTKSGKIVELAVRDVVHGRPVKNKEALANPEALDLFAGLDALKS

SEQ ID NO: 51 - DNA, 411 bp, Artificial Sequence
  HiybgC codon-optimized coding sequence
atgttagacaacggcttcagttttcccgtccgggtttactacgaggatactgatgcgggaggggtcgtttatcacgcaagatatttgcacttttttgaaagagccagaaccgaatacctccgtacgttgaacttcacacaacaaacattgcttgaggagcaacagttggctttcgtcgtaaagacactcgctatcgactattgtgtggcagccaagttggatgatttgctaatggtagaaactgaagtctcagaagttaagggagccaccattttgttcgaacagcgacttatgaggaatacgctaatgttatccaaggctaccgtgaaagtggcgtgcgttgaccttggtaagatgaaacctgttgcatttccaaaggaggtgaaagctgcatttcatcatcttaaatga

SEQ ID NO: 52 - DNA, 411 bp, Artificial Sequence
  HhybgC codon-optimized coding sequence
atgtccgagaaaacattttctttgcctgtcagagtttactacgaggatacggacgctggaggggtcgtatatcacgctagatatctccatttctttgaaagggcgcgaaccgaatatcttcgtgcattaaacttcactcaacagaccttgctccaagagcagcaattggcgtttgtcgttaagagtttggcaattgactactgtgtggctgccaagctagatgaccttcttactgtcgaaaccgatgttatagaaatcaaaggtgcaacaattttgttcgaacaacgattaatgcgggatacagttatgttgagcaaggccacggtgaaagttgcctcagtgcatcttggcaatatgaagccagtggcattgcccaaggaggtaaaggctgccttttacaacctaaaatga

SEQ ID NO: 53 - DNA, 408 bp, Artificial Sequence
  HpybgC codon-optimized coding sequence
atgttgaataaccaatgctttacatttccggtcagagtttattatgaagatacggatgcaggcggcgttgtttatcatgcacgttatttgcacttctttgaaagggcccgaacggagtatttgagaactttgaacttttcccagcagtctcttctccaagagcaacaagttgcctttgtcgtgaagacaatgtccatcgactattgtatcccagcaaagctagacgactaccttatagtcgagacagagattacagcggttaagggagcgactattttatttgaacaacgtttagtccgagagactgtgatgcttagtaaagctacggttaaggtagcatgcgttgatcttggtaaaatgaaaccagttgccattccagaagaactcaaagccgctttcctaaagtga

SEQ ID NO: 54 - DNA, 423 bp, Artificial Sequence
  RtybgC codon-optimized coding sequence
atggattcgaaaattttcagattcccagtaagggtctactacgaggacaccgatgctggcggtgtagtttaccacgcccgatatttgcattttttcgaaagggcccgtactgagtacttgcgtaagcaaaacttctcccaacaagtgctaatgcaagaacagcaacacgcctttgtagtcaagtctatggtcatagactattgcgttccagccaaattggatgattacttgatcgtggaaacagagttaacggcgattaagggtgcaaccttgctctttgaacaacgattgatgagggaggcaatcatgttgtctaaggccacagtcaaggtagcttacgttgacctcggcaagatgaagcctgctgctattccccgagaaatcaaatccgccttcagtaatgatatcacattctcagagtga

SEQ ID NO: 55 - DNA, 411 bp, Artificial Sequence
  RmybgC codon-optimized coding sequence
atggacagtaagattttcagatttcccattagagtttactacgaagatacagacgcgggtggtgtcgtttaccacgctcgttatctacattttttcgaaagagcccgtaccgaatacttgcgaaaacaagatttctcacaacagaagttactccaggagcaacaggcggcttttgtggtcaaggcaatgaccattgactattgtgtccccgccaaattagatgattatttgatcgttgaaacagaggtcacagccattaagggcgctacaattttattcgagcagaggttgttgcgagaaacagtgattttaagtaaggccaccgttaaggttgcttacgttgatcttggaaagatgaagcccacagcgattccagccgaaattaaggcggcattctcaaataacatctga

SEQ ID NO: 56 - DNA, 423 bp, Artificial Sequence
  RgYBGC codon-optimized coding sequence
atggtcagtaaagtgtttagattcccagtgcgagtttactacgaggacaccgatgcagggggagtggtttaccatgcacgatatctccacttttttgaaagagcaagaaccgaatatctcagaaagcaaaacttcagtcaacagatactattgcaggaacaacaggcagcatttgtagttaaatctatggccattgactattgtattccagccaagctagatgactatttgattgttgaaaccgaggtgactgcaattaaaggggcaacgatcttgtttgagcagagattgatgagagagacagtgattctaagtaaagcaacagtgaaagttgcttacgtcgatctcgggaaaatgaagccggcagcgatcccaacagagatcaaagcggcattcagcaacaacataacttttagtgagtga

SEQ ID NO: 57 - DNA, 741 bp, Artificial Sequence
  BxSrfAD codon-optimized coding sequence
atgggccaactctttaagtcgtttgacgcctccgaaaaaacacagctaatctgttttccgttcgctggagggtactcagcatccttccgacctttgcatgccttcctccaaggagaatgtgaaatgctagccgcggaacctccaggccacgggaccaaccagacgtccgcgatagaggacttggaagagttaactgatctttataagcaagagttgaatctcagaccagatagaccgtttgttttatttggacactcaatgggtgggatgattacatttagattggctcagaagttggagagagaaggtatattccctcaagctgttattatttccgctatccagccaccacacatacaacgtaagaaagtctcacacttgccagacgatcaattcctagatcatatcatccaattgggaggcatgccagcggagttagttgaaaacaaggaggttatgtcattcttcttgccgtcattcaggtctgattatagggcattggaacaattcgaattgtatgacctagcccagatccaatctccagtacatgttttcaacggcttagatgacaaaaaatgtataagggacgccgaagggtggaagaaatgggccaaagacattacttttcaccagtttgatggtggacatatgtttttactatcacaaacggaggaggtcgcagaaagaatatttgctatccttaaccagcacccgatcatccaacccgggtcaagatcctga

SEQ ID NO: 58 - DNA, 735 bp, Artificial Sequence
  BaSrfAD codon-optimized coding sequence
atgagccagttattcaggtcattcgatactagtgaaaagacccagttgatatgctttcctttcgccggtggttacagtgcttcatttaggccattacacgcatatctacaaggcgaatgtgaaatgatggctgcagagccaccaggccacgggactaaccaaactagcgcgatcgaggatttggaggagcttgttaggctttacaaacaggaattgaacctccgtccagatagaccattcgtactcttcggccacagcatgggcggaatgattactttcagacttgcccaaaaacttgaacgtgaggggatcttcccgcaagcagttattatttcagcaattcagccaccaaacgtcaagcgtaaaaaagtttcgcatcttgaggatgaagcgtttcttgaacacataatggaattgggcggcatgcccgctgaattagtcgagaacaaggaggtgatgtctttcttcctcccatcgttccgaagtgactatagagcattagagcgttttgaacctagggatctagggcagctaaagtcaccggtgcacatcttcaatggccgaaaagatgaaaaatgtatgagagatgcccatggttgggaccgatgggcgcagaacccagtgtatcatgaattcgatggaggtcacatgttcctattgagtgaaaccgaaaaagtagccgaagaaatattttctatcttgaatcattgcaagcccgcagtttcacataaatga

SEQ ID NO: 59 - DNA, 729 bp, Artificial Sequence
  BhSrfAD codon-optimized coding sequence
atgagtcaactattcaagagtttcgacgcaagcgaaaaaacgcaacttatctgtttcccgtttgcgggtggctattcagcatcatttagaccattacatgcatttcttcagggggaatgcgaaatgttggcagcggaaccgcccggtcacggtacgaatcagactagcgcaattgaggatttggagcagttaactgatttgtacaagcaagaactacacctccacccagatcgaccatttgtcttgtttggtcattccatgggaggaatgattacctttagacttgcccaaaaattggaacgtgaaggcatctttccgcaggctgtgattatcagtgccatccaacccccacatatccagcgtaagaaggtgtctcaccttccagatgaccaattcttggaccatatcattaagctaggtggaatgccagccgaattggtggaaaataaagaagttatgtcattttttctaccatcatttagatcggactatagggcacttgaacagtttgagctaaacgacatgacgccaatccaatcgcccgtgcatgtgttcaacgggctaaacgacgagaaatgtatccgtgacgctgaagggtggaagaagtgggcaaaacatattatgtaccatgagttcgacgggggtcacatgttcctactttccaacacggaagaggtagcagagagaatctttgctatcttgaaccaacatccaatttcaagtagttga

SEQ ID NO: 60 - DNA, 1953 bp, Artificial Sequence
  SmACL codon-optimized coding sequence
atgcaggcagaaagaccgctttgggtgccagatagagaaatagtcgagcgtagtcccatggcagaatttatcgattggtgtggagagcgttttggccgttcatttgcagactatgatgcatttcacgactggtcggtgtcagagagaggtgcgttctggaccgctgtgtgggagcactgtaaggtcattggtgagtccggcgaaaaggcacttgtggacggtgatcgtatgttggatgcaagattttttcccgaggcacgtcttaactttgccgaaaatttgttgagaaaaacgggaagcggcgatgccctcatctttagaggagaggacaaggtttcgtacagattgacgtgggacgagttgagagctttagtctcccgattacaacaggccctacgtgcacaggggattggggcaggcgaccgagtggccgcaatgatgcctaatatgccagagacaatcgcgttgatgctagcgacagcttccgtcggcgctatttggtcatcctgctcacctgacttcggggaacagggagttttggatagatttggtcaaatcgcaccaaagttgttcatcgtttgtgacggttactggtacaatggaaaacgtcaggacgttgactcaaaggtccgagccgttgcgaaaagccttggggcaccaacggtgatcgtaccgtacgcaggtgatagtgctgccttggcccctactgtagaggggggtgtcacgcttgccgatttcattgctggattccaagctgggccattggtgttcgagagattgcccttcgggcatcccctttatattttgttttcttccggcaccactggtgtacccaagtgcatagttcactcagcaggtggtacattgcttcaacatcttaaggaacatagattccattgtggattgagggatggtgagcgtttgttttatttcacaacttgtggatggatgatgtggaactggctcgcttcaggccttgctgtcggtgcaactctatgtctttatgatggcagccccttctgtcccgacgggaatgtgctattcgactacgctgccgccgaacgatttgcagtgtttggcacatcggccaagtacattgatgctgttagaaaagggggttttacgccagcgcgaactcacgatcttagctccctaagattgatgacatcaacaggttccccgttgtctccagaaggattttcatttgtttacgaaggaataaagccggacgtgcaattggcatcaatctctggcgggactgacatcgtttcctgtttcgtactcggtaatccactaaaaccggtttggagaggagaaatacaaggaccggggttgggcctcgctgtagatgtttggaatgatgaaggtaaaccagtacgaggagagaagggtgaattggtctgtacacgtgctttcccatctatgcctgttatgttctggaatgatccagatggcgccaagtacagagcggcttactttgatcgatttgacaacgtctggtgtcatggagacttcgctgagtggacgccgcacggcgggattgtgattcacggaaggtcagatgcaactttgaacccaggcggtgtcagaattgggactgccgaaatatataatcaagtcgagcaaatggacgaggtcgccgaagcattgtgcattggacaggattgggaagacgacgtgagagtggtattgttcgtgaggttggcgagaggggttgagttaactgaagcgttaacacgagaaataaaaaacagaatccgatcaggcgctagcccaagacatgttccggcaaaaattatcgctgttgcagacataccaaggacaaagtccgggaaaatcgtggaattggctgtgagggatgtagtgcatggaagacctgtgaaaaataaggaagccttagcaaatcctgaggcgctcgatcttttcgctgggttggaggagctcaaatcctga

SEQ ID NO: 61 - DNA, 1953 bp, Ensifer
atgcaagcggacagaccgttgtgggttccggatagcgccattctagaaagatcaccgatggcacaatttatgggctggtgtggtcaacgttttggaagaactttcgctgattacgacgccttccacgattggtctgtcgctcacagaggagacttttggacaggtttatgggaacattgtgacgttattggggaaaggggagaaagagctttgataaacggagaacgtatgttggacgcccgttttttcccagatgcaaagttgaactttgccgagaacttactaagaaaaagtggcggcggtgatgcgctaattttccgtggtgaagacaaggtgtcttatagattgtcctgggacgagttgcgagctttggtttcgaggctccagcaagcacttaagggacagggggtgggtgtaggagacagagttgctgcaatgatgccgaacatgccagaaacgattgcattgatgctagccacagcctccttgggagccatttggtcatcatgcagccccgattttggcgagcagggagtcttggacagattcggccaaatagggccaaagttgttcattgcatgcgacggatactggtataacggcaagcgacaggatgtggacgcgaaggttagggcggtggcgaaggccttgggggtgcctactgtgattgttccatacgctggagatagtgccgctcttgctgccgtgattgagggtggtgccactttgccagattttattgccggtttcgacgcgaagccccttgcttttgaacgacttcctttcggacaacccttgtacattcttttcagctcggggaccaccggggtgccaaaatgtatagtccattcggccggaggcaccctccttcaacacctcaaggaacacagatttcactgtgggcttaaagaaggtgaacgactcttctacttcagcacgtgcggatggatgatgtggaactggttggcttcaggattagcggccggggctacactttgtctctacgatggatcaccattctgccccgacggaaatgtactctttgattatgcggcagctgagaggtttgcagttttcggaacctcggcaaagtatattgatgcagtgcgaaaagctggcctcacacctgctaagactcacgacctcgctagtttgagactcattactagtaccgggtccccactaagtcctgaaggattcagcttcgtctacgaagggataaaaccagatgtgcaattagcaagtatctcaggcggtactgacatcgtatcgtgttttgtcctaggcaatccgttgaaaccggtctggagaggagagatacaggggcccgggctaggtttggccgttgacgtttggaatgatgaaggccagccagttagaggtgaaaaaggagaacttgtgtgtactaaggccttcccaagtatgccagtgatgttctggaatgatccagatggtgctaaataccgagcagcctattttgagcgttttgacaacgtttggtgtcatggagattttgccgagtggacggcacacggtgggataatcattcatggaaggtcagatgcaacactcaacccgggtggagttaggatcggaacagcagaaatttataaccaagttgaacaaatgcctgaagttttggaagccttgtgcataggtcaggattgggaagatgatgtccgtgttatattgttcgtccgacttgcgcccggagtggaattaacagaggagcttactaaagccatcaagaaccgtattagaataggcgcatcacccaggcatgtcccagcaaaggtgatcgcggtagcagacatacctcgaactaagtccggtaagatagtagaattggcagtacgagatgttgttcacggaaggcccgtgaagaacaaggaggcgttggcaaaccccgaagcgctcgatttgtttgccgaattggacgccttgaagtcctga

SEQ ID NO: 62 - DNA, 1953 bp, Sinorhizobium fredii
atgcaaacagacagaccgctttgggtcccggacgcagaaacgctcgcccgttcaccaatggcggagtttatggcctggtgtgaggaaagattcggcagaaggttttccgactacgatgcattccatcaatggagtgtcactgaaaggggcgacttttggactgcagtatgggaacattgcggcgtaatcggtgagcgaggggatcaagcgttgatcaatggagagcgtatgctagaggcccgtttctttcctgaagcaaaattgaacttcgcggagaatctactacgaggctcgggagatggtgacgcattaattttcaggggtgaagacaaagttaggtacagactcagttgggatgaattaagaagtttggtgtcaaggcttcaacaagctctaaaggcccaaggcattggcgtgggggaccgagtggctgcaatgatgccaaaccttccagaaaccattgctttgatgttggcgaccgcgtcggtcggtgcaatttggtcgtcctgttcaccagattttggtgagcagggagtcctcgacagatttggtcaaattggacccaagcttttcgttgtttgtgacggctactggtacaatggaaaacgtcaaaacgtggatgcaaaagtaagagcggtcgctaaaacccttggggtcccgaccttggttgtgccatacgcaggggacagcccgggccttgccgctgctatcgatggaggagtaaccttgccagattttattgcaggtttcgaagcaaagccattagaattcgaaagattgccatttggtcatccattgtacattttgttttcaagcggcaccacgggtgtccctaaatgtatcgtacatagcgctggaggcacacttcttcagcatctaaaagagcatcgatttcattgcggtttgaaagaaggtgagcgacttttttactttacgacttgcggatggatgatgtggaactggctagcttccggactcgctgtaggggcaaccctatgtttgtatgatggaagtcccttttaccctgatggaaacgttttgtttgatttcgcagcagcagaacgtttcgctgtttttggcacaagtgccaagtacattgatgcagttagaaagggggggttgacgccagccacaactcacgacttgccagccttgaggctccttacttctaccgggtcacctttgtctcccgaaggtttttcctttgtttatgaagggattaaatcggatgtgcaacttgccagcatttctggcggaaccgacattgtgtcatgtttcgtcctcggaaacccattgaaaccggtatggagaggtgaaattcagggtccagggctcggattggcggtcgatgtttggaacgatgaaggaaagccggtcagaggtgaaaagggagaattagtgtgtacaaaggccttcccctccatgcctgtaatgttctggaatgatcccgagggcgcaaagtatagagccgcgtacttcgagcgatttgataatatctggtgtcatggggatttcgctgagtggactccacacgatggcatcgttattcatggtagatcagatgccactctaaacccgggtggtgttagaatcggtacggcggagatatacaaccaagtggaacaaatgcctgaggtggcagaagcattgtgtattggacaggattggcaagacgacgttagagtgattctttttgttagattggccccaggcgtagccctaaccgaagaattatcccaagaaattaagacacgaatcaggaccggagcctcccctcgacatgttcccgccaaaattattccagtgtccgacatccctcgtacgaagtctggaaagattgttgagttggcggtcagggacgtcgttcatggacgtccggtaaaaaacaaggaggcgttggcaaacccagaagctttggatctattcgccgggctcgacgcactaaagtcttga

SEQ ID NO: 63 - protein, 246 aa, Clostridium beijerinckii
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 64 - protein, 246 aa, Clostridium beijerinckii
MLESEVSKQITTPLAAPAFPRGPYRFHNREYLNIIYRTDLDSLRKIVPEPLELDGAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 65 - protein, 246 aa, Clostridium beijerinckii
MLESEVSKQITTPLAAPAFPRGPYRFHNREYLNIIYRTDLDALRKIVPEPLELDGAYVRFEMMAMPDTTGLGSYTECGQAIPVKYNEVKGDYLHMMYLDNEPAIAVGRESSAYPKKLGYPKLFVDSDTLVGTLKYGTLPVVTATMGYKHEPLDLKEAYTQIARPNFMLKIIQGYDGKPRICELICAENTDITIHGAWTGSARLQLFSHALAPLADLPVLEIVSASHILTDLTLGTPKVVHDYLSVK

SEQ ID NO: 66 - protein, 252 aa, Artificial Sequence
  CbADC1-P
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVKGRRAKL

SEQ ID NO: 67 - protein, 248 aa, Clostridium sp. BL-8
MLETEVIKQITTPISAPAFPRGPYKFHNREYLNIIYRTDLEALRKIVPEPLELDGPYVRFEMMAMPDTTGLGSYTECGQAIPVTFNGVSGDYLHMMYLDNEPAIAVGRESSAYPKKLGNPKLFVDSDTLVGTLKYGTLPVATATMGYKHEPLDLKEAYSQISRPNFMLKIIQGYDGKPRICELICAENTDITIHGAWTGSARLQLFSHALAPLADLPVLEIISASHILTDLTLGTPKIVYDYISKNQK

SEQ ID NO: 68 - protein, 248 aa, Clostridium gasigenes
MLESEIYKQITTPLTAPAFPKGPYKFHNREYLNIIYRTDFEALRKIVPEPLELDEDEPLVRFEMMAMPDVTGLGSYTECGQVIPVKYNGIKGDYLHMMYLDNHPAIAVGREVSAYPKKLGTPKLFVDSDTLVGTLNYGSLPVATATMGYKHEPLDIKEAYNQISRPSFMLKIVQGYDGKPRICELICTQNKDIIIHGAWTSCARLQLFSHALAPLADLPVLEVVSASHILTDLTLAPAHVVYDYLAKN

SEQ ID NO: 69 - protein, 245 aa, Clostridium acidisoli
MLESEVSKQITMPLTAPAFPRGPYRFHNREYLNIVYRTNADALRKAIPEPLTFDEPLVRFEMMAMPDVSGLGSYTECGQVIPVNFNGKKGDYLNMMYLDNHPAIAVGRELSSYPKKLGYPKLFVDSDTLVGTLDYGTLRVLTATMGYKHQSLDINTARNEICRPNFMLKIIPNYDGSPRICELVCTQITDITIHEAWTGPARLQLFDHAMAPFNDLPVKEIISSSHILTDLTLPTPTVVYDYLKK

SEQ ID NO: 70 - protein, 247 aa, Paenibacillus sp. ov031
MRKEEVVRQFTTPLDAGAFPLGPYRFYNREYLNILYRTDLESLRKIVPEPLEVTSPLVRFEIMKMPDVTGLGSYTECGQVIPVNFEGEEGDYLHAMYVDSFPAIASGREIGAYPKKMGKPSLYVDSDTLVGLMDYGSLRVATATMGYKHFPLDHSEALQEIVKPNYMLKKMPDYDGYPRICELVRSQITDIKIQGAWSGPARLELFAHALAPMADLPVLEVVSASHIITELSLPSPKVIYDYLADEK

SEQ ID NO: 71 - protein, 246 aa, Heyndrickx sporothermodurans
MKKEDVVKQFTTPLDAGAFPLGPYRFYNREYLNILYRTDLESLKKIVPEPLEVKDPLVRFEIMKMPDVTGLGSYTECGQVIPVSFEGEEGDYLHAMYVDSFPAIASGREIAAYPKKLGKPSLYVDSDTLVGLLDYGSLRVATATMGYKHFPLDHSEALKEIVKPNYMLKKMPDYNGYPRICELVRSEITDIKILGAWSGPARLELFAHALAPMADLPVLEVVSASHIITELSLASPTVIYDYLAEK

SEQ ID NO: 72 - protein, 245 aa, Variovorax sp. KK3
MKIEELFKQVSTPLGAPAFPRGPYRFHNREYLNITYRTDLEALRKVVPEPLEVDEPLVRFEVMRMPDTTGLGNYTECGQAIMVRLGQERGEYLHAMYVDNHPAIASGREVSAFPKKLGAPKLYVDSDTLVGTLDYGTLRIAVATMGYKHQALPVEEARAEICVPTFMLKLMRGYDRQLRICELVRSQIQDIEIKTAYKGPARLQLFEHVLAPMADFPVREVVAASHILADLSLTPPSVVHDYLAK

SEQ ID NO: 73 - protein, 246 aa, Clostridium cagae
MLKSEVSKQITTPLTAPAFPRGPYRFHNREYFNIIYRTDADALRKIVPEPLELGEDPLVRFEMMAMPDTSGLGSYTECGQAIPVSYNGKKGDYLHMMYLDNEPAIAVGRELSAYPKKLGYPKLFVDSDTLVGTLDYGKLRVAIATMGYKHKQLDLNEAKEQICRPNFMLKIIPNYDGTPRICELISAQIKDITVHEAWTGPARLQLFDHAMAPFNDLPVKEIVSSSHILTDLTLPSPEVIYDYLKK

SEQ ID NO: 74 - protein, 246 aa, Clostridium pasteurianum
MLKSEVSKQISMPLTAPAFPRGPYKFHNREYLNIVYRTDANALRKVVPEPLELNEPLVRFEMMAMPDVSGLGSYTECGQVIPVSFNGKKGDYLHMMYLDNHPAIAVGRELSSYPKKLGYPKLFVDSDTLVGTLDYGKLRVLTATMGYKHQSLDINEARNQICSPNYMLKIIPNYDGTPRICELVCAQITDITIHEAWTGPARLQLFDHAMAPFNDLPVKEIVSSSHILTDLTLPAPEVVYDYLKNS

SEQ ID NO: 75 - protein, 246 aa, Priestia megaterium
MQQDEVIKQFTTPLGAPAFPVGPYRFHNREYLNILYRTDIDSLRKIVPEPLEITDPLVRFEIMKMPDVTGLGSYTECGQVIPVSFNGEEGEYLHAMYVDSHPAIASGREIGAYPKKLGKPSLFVDSDTLVGTLDYGSLRVATATMGYKHCPLDLSEARKEICKPSYMIKKMPGYDGYPRICELVRSQITDITIHGAWSGPARLELFAHALAPMSDLPVYEVVSTSHILTDLTLASPRVVHDYLASK

SEQ ID NO: 76 - protein, 246 aa, Bacillus acidicola
MLKKDVRRQITTPLGGPAFPPGPYKFHNREYLNIVYRTDLDALRRIVPEPLEIEEPLVKFEIMYMPDVTGLGCYTESGQVIPVKFNGVEGEYLHSMYVDNHPAIAGGREASAYPKKLGAPRLFIDSDTLVGTLDYGSLRVATATMGYKHYPLDIEKAREEITKPTFMLKIVPNFDGTPKVCELVQTQITDLTIHGAWTAPARLQLFEHALAPMADLPVLEVVHASHILTDLTLSAAVPVYNYLNEK

SEQ ID NO: 77 - protein, 247 aa, Pelosinus fermentans
MLKSEVLKCFTTPLTGPAFPKTTYRFNNREYLNIVYRTDLTALRRVVPEPLEIIDPLVRFEMMRMPDVTGLGSYTESGQVIPVRFNGEEGDYLHAMYLDNLPAIAFGREVSAFPKLLATPKLYVDNDTLVGTLDYGSLRVATATMGYKHFPIDQTEAFAEIGRPTFMIKIMRGYTGEPKVCELVRTQTTDITIHGAWKGPARLQLFAHALAPLADLPVLEVVGASHILTDLTLAPVKMVYDYLAEKK

SEQ ID NO: 78 - protein, 248 aa, Clostridium estertheticum
MLESEISKQITTPLTAPAFPKGPYKFHNREYLNILYRTNLEALRKMVPEPLELDEDEPLVRFEMMAMPDVTGLGSYTECGQVIPVKYNGIKGDYLHMMYLDNHPAIAVGREISAYPKKLGTPKLFVDSDTLVGTLNYGSLRVATATMGYKHEPLDIKEAYNQISRPSFMLKILQGYDGKPRICEIICTQNKDITIHGAWTGCARLQLFSHALAPLADLQVLEVVSASHILTDLTLASPHVVYDYLAKN

SEQ ID NO: 79 - protein, 248 aa, Clostridium estertheticum
MLESEISKQITTPLTAPAFPKGPYKFHNREYLSILYRTNFVALRKMVPEPLELDEDEPLVRFEMMAMPDVTGLGAYTECGQIIPVKYNGIKGDYLHMMYLDNHPAIAVGRELSAYPKKLGTPKLFVDSDTLVGTLNYGSLRVANATMGYKHEPLDIKEAYNQISRPIFMLKILQGYDGKPRICEIICTQNKDITIHGAWTGCARLQLFSHALAPLADLQVLEVVSASHILTDLTLGSPYVVYDYLAKN

SEQ ID NO: 80 - protein, 246 aa, Brevibacterium
MQQDEVIKQFTTPLDAPAFPVGPYRFHNREYLNILYRTDLDALRKIVPEPLEITDSLVRFEVMKMPDVTGLGSYTECGQVIPVSFDGEEGEYLHAMYVDSFPAIASGREISAYPKKLGKPNLFVDSDTLVGTLDYGSLRVATATMGYKHRPLDLSEARKEICKPNFMIKKMPGYDGYPRICELVRSQITDITIHGAWSGPARLELFAHALAPMSDLPVLEVVSTSHIITDLTLSSPSVVHDYLTSK

SEQ ID NO: 81 - protein, 248 aa, Clostridium sp DL-VIII
MLENEVIKQITTPISAPAFPRGPYKFHNREYLNIIYRTDLEALRKIVPEPLELDGPYVRFEMMAMPDTTGLGSYTECGQAIPVTFNGVSGDYLHMMYLDNEPAIAVGRESSAYPKKLGNPKLFVDSDTLVGTLKYGTLPVATATMGYKHEPLDLKEAYSQISRPNFMLKVIQGYDGKPRICELICAENTDITIHGAWTGSARLQLFSHALAPLADLPVLEIISASHILTDLTLGTPKIVYDYLSKNQK

SEQ ID NO: 82 - protein, 246 aa, Clostridium saccharoperbutylacetonicum
MLESEVSKQITTPLAAPAFPRGPYRFHNREYLNIIYRTDLDALRKIVPEPLELDGAYVRFEMMAMPDTTGLGSYTECGQAIPVKYNGIKGDYLHMMYLDNEPAIAVGRESSAYPKKLGYPKLFVDSDTLVGTLKYGTLPVATATMGYKHEPLDLKEAYNQIARPNFMLKIIQGYDGKPRICELICAENTDITIHGAWTGSARLQLFSHALAPLADLPVLEIVSASHILTDLTLGTPKVVHDYLSLK

SEQ ID NO: 83 - DNA, 741 bp, Artificial Sequence
  CbADC1 codon-optimized coding sequence
atgttggaatcagaagtttcaaagcaaattacaacaccattggcagcaccagcatttccaagaggaccatacagattccataacagagagtacttgaacattatttacagaacagatttggatgcattgagaaagattgttccagaaccattggaattggatagagcatacgttagatttgaaatgatggcaatgccagatacaacaggattgggatcatacacagaatgtggacaagcaattccagttaagtacaacggagttaagggagattacttgcatatgatgtacttggataacgaaccagcaattgcagttggaagagaatcatcagcatacccaaagaagttgggatacccaaagttgtttgttgattcagatacattggttggaacattgaagtacggaacattgccagttgcaacagcaacaatgggatacaagcatgaaccattggatttgaaggaagcatacgcacaaattgcaagaccaaactttatgttgaagattattcaaggatacgatggaaagccaagaatttgtgagttgatttgtgcagaaaacacagatattacaattcatggagcatggacaggatcagcaagattgcaattgttttcacatgcattggcaccattggctgatttgccagttttggaaattgtttcagcatcacatattttgacagatttgacattgggaacaccaaaggttgttcatgattacttgtcagttaagtga

SEQ ID NO: 84 - DNA, 741 bp, Clostridium beijerinckii
atgttagaaagtgaagtatctaaacaaattacaactccacttgctgctccagcgtttcctagaggaccatatagatttcacaatagagaatatctaaacattatttatcgaactgatttagattctcttcgaaaaatagtaccagagccacttgaattagatggagcatatgttagatttgagatgatggctatgcctgatacaaccggactaggctcatacactgagtgtggtcaagccattccagtaaaatataatggggttaaaggtgactacttgcatatgatgtacctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 85 - DNA, 741 bp, Clostridium beijerinckii
atgttagaaagtgaagtatctaaacaaattacaactccacttgctgctccagcgtttcctagaggaccatatagatttcacaatagagaatatctaaacattatttatcgaactgatttagatgctcttcgaaaaatagtaccagagccacttgaattagatggagcatatgttaggtttgagatgatggctatgcctgatacaaccggactaggctcatatactgagtgtggtcaagccattccagtaaaatataatgaggttaaaggtgactacttgcatatgatgtacctagataatgaacctgctattgctgttggaagagaaagcagtgcttatcccaaaaagctcggctatccaaagctatttgttgattcagacaccctagttggcacccttaagtatggtacattaccagtagttactgcaacaatgggatataagcatgagcccctagatcttaaagaagcctatactcaaattgcaagacccaatttcatgctaaaaatcattcaaggttatgatggtaagccaagaatttgtgaactcatctgtgcagaaaatactgatataactatccacggtgcttggactggaagtgcacgtctacaattatttagccatgcactagctcctcttgctgatttacctgtattagagatcgtatcagcatctcatatcctaacagatttaactcttggaacacctaaggttgtacatgattatctttcagtaaaataa

SEQ ID NO: 86 - DNA, 759 bp, Artificial Sequence
  CbADC1-P codon-optimized coding sequence
atgttggaatcagaagtttcaaagcaaattacaacaccattggcagcaccagcatttccaagaggaccatacagatttcataacagagaatacttgaacattatttacagaacagatttggatgcattgagaaagattgttccagaaccattggaattggatagagcatacgttagatttgaaatgatggcaatgccagatacaacaggattgggatcatacacagaatgtggacaagcaattccagttaagtacaacggagttaagggagattacttgcatatgatgtacttggataacgaaccagcaattgcagttggaagagaatcatcagcatacccaaagaagttgggatacccaaagttgtttgttgattcagatacattggttggaacattgaagtacggaacattgccagttgcaacagcaacaatgggatacaagcatgaaccattggatttgaaggaagcatacgcacaaattgcacgaccaaactttatgttgaagattattcaaggatacgatggaaagccaagaatctgtgaattgatttgtgcagaaaacacagatattacaattcatggagcatggacaggatcagcaagattgcaattgttttcacatgcattggcaccattggctgatttgccagttttggaaattgtttcagcatcacatattttgacagatttgacattgggaacaccaaaggtggttcatgattacttgtcagttaagggtagacgagcaaagctttga

SEQ ID NO: 87 - DNA, 747 bp, Clostridium sp. BL-8
atgctagaaactgaagtaataaaacaaattacaactcccatatctgctccagcatttcctagaggaccatataaatttcataatagagaatatttaaatatcatttatcgtactgatttagaagctcttagaaaaatagttccagaaccacttgagttagacggcccttatgtgagatttgaaatgatggctatgccggatacaactggattgggctcatatacagaatgtggtcaggctatcccagtaacattcaatggagttagcggagattacttgcatatgatgtaccttgataatgagccagctatagctgttggaagagaaagtagcgcttatcctaaaaaacttggaaatccgaagttatttgttgattcagatacattggttgggacattaaaatatggcacattgccagtggccactgccacaatgggatataaacatgaacctctagatcttaaagaagcttattcacaaatttcaaggcctaattttatgttaaaaattattcaaggttatgatggtaagccaagaatttgtgaacttatatgcgcagaaaatactgatataacaatacatggtgcatggactggtagcgctcgtctacaattgtttagccacgcactagctcctttagctgacttaccagtattagaaattatatctgcatcacatatacttacagatttaactcttggaacacctaaaattgtttatgattatatttccaaaaatcaaaaatga

SEQ ID NO: 88 - DNA, 747 bp, Clostridium gasigenes
atgttagaaagtgaaatatataaacaaattacaacaccacttactgctccagcatttcctaaaggtccatataagtttcataatagggagtacttgaacataatataccgtactgactttgaggcacttcgaaaaatagtaccagaaccattagaattagatgaagatgagcccttggttagatttgaaatgatggctatgccagatgtgactggattgggatcctacactgaatgtggtcaggtaattccagtaaagtataatggaattaaaggtgattatctacatatgatgtacttagacaatcatccagctatcgctgttggaagagaggttagtgcctatcctaagaaacttgggactccaaagctatttgttgactcagataccttagttggtacattaaactatggatctttaccagttgctactgctacaatgggatataaacatgaacctctagatattaaggaagcttataatcaaatttctcgtcctagctttatgctgaaaattgtacaaggatatgatgggaaaccaagaatttgtgagcttatatgtacacagaataaggatattatcatacatggggcatggactagttgcgcacgtctgcaattatttagtcatgcattagctcctttggctgatttaccagtattagaagttgtatctgcatcacatattcttacagatttaactcttgcaccagctcatgttgtgtacgattatcttgcaaagaattag

SEQ ID NO: 89 - DNA, 738 bp, Clostridium acidisoli
atgttagaaagtgaagtatctaaacaaattacaatgccactaactgctccagcatttcctaggggaccttatagatttcataatcgtgaatatcttaatattgtctatcgtacaaatgctgatgctttacgtaaggccataccagagcccttaacttttgatgaacctttagttagatttgaaatgatggctatgcctgatgtaagtggtctaggttcctatactgaatgtggacaggttattccagtgaattttaatggtaaaaagggtgattatttaaatatgatgtatttagataatcaccccgctattgctgttggaagggagcttagttcatatcctaaaaagctaggctatccaaaattgtttgtcgactcagacacgttagttggtacattagactatggaacactaagagtgttaactgccactatgggatataaacatcaatctctagatataaatacagcgcgtaacgaaatttgtcgccctaattttatgttgaaaattatacctaattatgatggaagccccagaatttgcgaactagtatgtacacaaattacagatatcaccatacatgaggcatggacaggaccagcaagattacaattatttgatcatgctatggcaccatttaatgatttacctgtaaaagaaataatatctagctctcatattcttacggacttaactctcccaactcctacagttgtatatgattacctaaaaaaatag

SEQ ID NO: 90 - DNA, 744 bp, Paenibacillus sp. ov031
atgagaaaggaagaagtggttaggcaatttacaactccgctggatgcaggggcatttcctttgggaccatatcgtttctataatcgcgaatacttgaacattttatatagaactgatctggagagcttaagaaaaatcgttccggagccattggaagttacctctccgcttgtaaggtttgagatcatgaaaatgccggatgtaacaggccttggttcctatacagagtgtggacaggtgatacctgttaactttgaaggggaggaaggagattatctccacgcaatgtacgtagacagttttcctgcaatcgcatcaggcagggaaataggtgcctatcctaaaaaaatgggcaaacccagtttgtatgtagattccgacacattggttggacttatggattatggctctttaagagtggctaccgcgaccatgggatataagcattttccacttgatcattcggaggcgctacaagagattgttaaaccaaattatatgctgaaaaagatgcctgattatgacggatatccaagaatctgtgaactggtacggagccaaattacggatattaaaattcagggcgcctggtcaggtcctgcaagactggagctattcgctcatgctcttgcgccaatggcagatttgcctgtacttgaggttgtttcagcttcccatatcataacagagctttcgctgccttctccaaaagttatatatgattatttagctgatgaaaaataa

SEQ ID NO: 91 - DNA, 741 bp, Heyndrickx sporothermodurans
atgaaaaaggaagacgtggtaaagcagtttacaactccgttagatgcaggggcgtttccattaggaccgtatcgtttttataatcgcgaatacttaaacattttatatagaaccgacctggaaagcttaaagaaaattgtgccggaaccattggaggtgaaagatccgcttgtcagatttgaaatcatgaagatgccggatgtgacagggcttggatcctacacagagtgtggacaagtcatacctgttagttttgaaggggaggaaggggattatctccacgcaatgtatgtagatagctttcctgccatcgcatcaggcagggaaatcgcagcgtatcctaaaaagctgggcaagcctagtttatatgtagattccgataccttagtgggactcttggactatggaagtttgcgagtagcaacagcaacgatggggtataagcattttccgcttgaccattcagaggcgcttaaagaaatcgttaaaccgaattatatgttgaaaaaaatgccggactataatggatatccaagaatttgcgaattggttcgtagcgaaattactgatattaaaattctcggtgcctggtcaggtcctgcaagactcgagctgttcgcccatgcgcttgcaccgatggctgatttgccagtactagaggtcgtatcagcttctcatattattacagagctttctttggcatctccaactgttatatacgattatttagctgaaaagtaa

SEQ ID NO: 92 - DNA, 738 bp, Variovorax sp. KK3
atgaagatcgaagaactgttcaagcaggtcagcacgccgctgggtgcaccggcttttccgcgcggcccttaccgctttcacaaccgcgaatacctcaacatcacctaccgcaccgacctggaggcgctgcgcaaggtggtgcccgagccgctcgaagtagacgagccgctggtgcgcttcgaggtcatgcgcatgcccgacaccaccgggctgggcaactacaccgaatgcggccaggccatcatggtgcgcctgggccaggagcgcggcgagtacctgcacgcgatgtacgtcgacaaccacccggccatcgccagcggccgcgaagtcagcgcctttccgaagaagctgggcgcgccgaagctctacgtcgactccgacacactggtcggcacgctcgactacggcaccctgcgcatcgcggtcgccaccatgggctacaagcaccaggcgcttccggtcgaagaggcccgcgccgagatctgcgtgccgaccttcatgctgaagctcatgcgcgggtacgaccggcagctgcgcatctgcgagctggtgcgctcgcagatccaggacatcgagatcaagacggcgtacaagggaccggcgcggctgcagctgttcgagcacgtgctggcgccgatggccgactttccggtacgcgaagtggtggcggccagccatatcctggcggacctgtcgctgacgccgccttccgttgtccatgactacctggcgaaatga

SEQ ID NO: 93 - DNA, 741 bp, Artificial Sequence
  CcADC1 codon-optimized coding sequence
atgttgaaatccgaggtgtccaaacagattacaactccactaaccgcacccgcattcccacgagggccctatagattccataatcgagagtacttcaatattatctatcgaaccgacgcagacgcattgagaaaaatcgtaccagagcctcttgaattgggagaagatcccttggtcagattcgaaatgatggcaatgccagatacctcaggattgggaagttacacagagtgcggtcaggcgattcctgtctcctataacggtaaaaaaggtgattatttgcatatgatgtatctagacaacgaaccggctatagcagtgggtcgtgaactatctgcgtacccaaaaaagcttggttacccaaagttgtttgtcgattccgataccttggttgggacgttggactacggaaagctaagggtcgccatcgccacgatgggctacaaacacaagcagttggatttgaatgaggccaaggagcagatatgtagaccgaattttatgctcaaaattattcccaactacgatgggacgccacgtatatgtgaattaatctccgcacaaattaaagatattacggttcacgaagcttggacgggaccggctagactccaattgttcgatcatgctatggctcccttcaacgatctcccagtcaaagagatagtttcatccagtcacattcttacagaccttacgttgccttctcccgaggtaatttacgactacttgaagaagtga

SEQ ID NO: 94 - DNA, 741 bp, Artificial Sequence
  CpADC1 codon-optimized coding sequence
atgttgaagagtgaggtttctaagcaaatatcaatgccattaacagccccagcattcccaagggggccatacaagtttcacaacagagaatacttgaacattgtgtatcgaactgatgcaaatgccttgcgaaaggtggttccagagccacttgaacttaatgaaccattagtgaggttcgaaatgatggctatgccagatgtatctggcttaggttcctacactgaatgcggccaagtgatcccagtcagctttaacgggaaaaagggtgattacttgcatatgatgtacttagacaatcaccctgcaattgccgtcggaagagaattgagttcatatccaaagaaattgggttatccaaagttattcgtagattcagatacgttggtcggaactctagactatggaaaattaagagttttgacagccactatgggttacaaacatcaatctctcgatattaacgaggctcgtaaccagatttgttcgccaaattacatgcttaagataataccaaattacgacggtaccccacgtatttgtgagcttgtttgcgcccagattacggacattacaatacatgaggcttggacaggtcccgctaggttgcagttgtttgaccacgctatggcaccattcaatgacttgccggtaaaggaaattgtttccagttcgcacattttgacagacttaaccttaccggcacctgaagtcgtctatgattacttaaagaactcatga

SEQ ID NO: 95 - DNA, 741 bp, Artificial Sequence
  PmADC1 codon-optimized coding sequence
atgcaacaggatgaagttataaaacaattcacaaccccattgggggctccagcttttccagtgggtccttatagatttcacaacagagagtacttaaatattttatatcgtaccgatatagactcattgcgtaagatcgttcccgaaccattggagatcaccgatccattggttagattcgaaattatgaagatgccagacgtgacgggacttgggtcctatacggagtgtggacaagttattccggtgtcgttcaatggcgaagagggcgagtacttgcatgctatgtacgtggattctcatccggccatcgcaagcggaagagaaatcggcgcttaccccaagaagttgggaaagccatcactatttgtagactcggacactcttgtggggacgctcgactatggatcacttcgagtcgcaaccgcaactatgggatacaaacattgtcctttggacttaagcgaagccagaaaggagatatgtaagccatcatatatgataaaaaagatgcctggatacgatggctacccacgaatttgtgagttggtgcgatcacaaataaccgacattaccattcacggagcttggtcaggtcccgctcgtttggagctttttgctcacgccttggccccaatgtccgacctcccagtatacgaagttgtcagtacctcacatatccttacagaccttacgttagctagcccaagggtagttcatgattatctagcttcgaaatga

SEQ ID NO: 96 - DNA, 741 bp, Artificial Sequence
  BaADC1 codon-optimized coding sequence
atgttgaaaaaggacgttagacgtcaaatcactacccctcttggtggtccggccttcccacccggtccgtataaatttcataatagagagtatttgaacattgtatacaggaccgacttggacgccctcaggcgtatagttccagagcctttagaaatcgaggagccacttgttaagtttgagataatgtacatgcccgacgtgacaggcttgggctgttacactgaatcaggccaggtcattcccgttaaatttaacggtgtggagggagaatacttacatagtatgtatgttgacaatcacccagcaatcgcaggtggacgagaagccagcgcttatcctaagaaacttggcgctccaagactctttattgattctgatactttggtcggcacccttgattacggctcgctcagagtggcaactgcaaccatgggatataagcattatccactagatatagagaaagccagggaagagataacgaaaccgaccttcatgctaaaaatagtacccaattttgatggtaccccaaaggtttgcgaattagttcagacacaaattacggacttgaccatccacggagcgtggactgcaccggccagattgcaattgttcgaacacgctttggcgccgatggcggatctaccagtccttgaagttgttcacgcaagccacatcttgacggacctaacgttgagcgcggcggtgcccgtttataattaccttaacgagaaatga

SEQ ID NO: 97 - DNA, 744 bp, Artificial Sequence
  PfADC1 codon-optimized coding sequence
atgcttaagagtgaagtattgaagtgctttacaacaccattgacaggaccagcttttccaaaaacaacttacagatttaataacagagagtacttaaatatagtctacaggacagacctcacggcattgagaagagtcgttcctgaaccgttggaaataattgacccgcttgtccgttttgagatgatgcgaatgccagacgtcacgggattgggttcctatacggaatctggtcaggttatccctgtgagattcaatggtgaagagggggattaccttcatgcaatgtacctcgataatctcccagccatagcattcggaagggaagttagtgcttttccaaagttgctcgctacgccgaaactatatgttgataacgacacacttgtgggaacattagattacggatcacttagagtggctacagccacaatggggtacaaacattttcctatcgatcaaacagaggcttttgccgaaatcggcaggcccacctttatgattaaaatcatgaggggatacacaggagaaccaaaggtgtgtgagttggtaagaacacaaacaacagatatcaccatccacggagcatggaaaggcccagctcgacttcagctttttgcgcatgcattggctccattggcagatttgccagttctcgaagttgtaggtgcttcacatatattgactgacttgaccttggctcctgtcaaaatggtgtatgattacctcgcggaaaaaaagtga

SEQ ID NO: 98 - DNA, 747 bp, Artificial Sequence
  CeADC1 codon-optimized coding sequence
atgttagagagcgagattagtaaacaaatcactaccccgctcacggcgcctgcgttccctaaggggccatacaaattccacaaccgtgaatatttgaacattctttacagaacgaatttggaagcactcaggaagatggtcccagagcctcttgagcttgatgaagatgagccactagtccgtttcgaaatgatggctatgccagacgtcaccggattagggtcctataccgagtgtggacaagtcatcccagttaagtataacggaataaaaggcgattatctacatatgatgtatcttgataaccatccagcaattgcagtggggcgagagatatccgcttacccaaaaaaattgggtacaccaaagctattcgtggacagcgacactctcgtggggactttgaactatggaagcttgagagttgccactgctacaatgggatacaaacatgagccattggatatcaaggaggcctataatcaaatatccagaccaagctttatgttgaagattttgcaagggtatgatggtaaaccaagaatatgtgaaatcatttgcacccagaataaagatattacgatccatggcgcatggacaggttgtgcgcgtttgcagttgttttcccatgctctagcgccacttgctgaccttcaggttttagaagtagtttcagcatcccatattttgactgatcttactctcgccagcccacacgtcgtctacgactatttggctaagaactga

SEQ ID NO: 99 - DNA, 747 bp, Clostridium estertheticum
atgttagaaagcgaaatctctaaacaaattacaacaccacttactgctccagcattcccaaaaggaccatataagttccataatagggagtacttaagtatactataccgtactaactttgtggcacttcggaaaatggtgccagaaccattagaattagatgaggatgagccactagttagatttgaaatgatggctatgccagatgttactggattgggagcctacactgaatgcggacagattattccagtaaagtataatgggattaaaggtgattatctgcatatgatgtacttagacaatcacccagccattgctgttggcagagagcttagtgcttatcccaagaaactgggtactccaaagctatttgttgactcagatacattagttggtacactaaattatggctcattacgagttgccaacgctaccatgggttataaacatgaacctctagatattaaggaagcttataatcaaatttctcgtcctatctttatgctgaaaattttacaaggctatgatggaaaaccaaggatttgtgaaattatctgtacacagaataaggatattaccatacatggtgcatggactggctgtgcacgtctgcaattatttagtcatgcattagctcctttagctgatttacaagtcttagaagttgtatctgcatcacatattcttacagatttaactcttggatcaccttatgttgtgtacgattatcttgcaaaaaattag

SEQ ID NO: 100 - DNA, 741 bp, Artificial Sequence
  BxADC1 codon-optimized coding sequence
atgcaacaagatgaagtgatcaaacaattcacaacgccgctagatgcacccgcattcccagttggtccttatagatttcacaaccgagaatatcttaatatattgtatagaaccgacttagacgccctcagaaagatagttcctgagccattagaaattaccgattctcttgttagatttgaggtgatgaagatgcccgacgtgactggtttgggatcatatactgaatgtggtcaagttattcctgtttcttttgatggagaggagggtgaatatcttcacgcgatgtacgtggacagttttcccgcaatcgcatctgggagagagatttctgcctacccaaagaaattaggtaaaccaaacttgtttgttgatagtgacacattggtcgggaccctagattatgggtcactcagagtagcgacggctactatggggtacaagcacagaccgcttgatctcagcgaagccagaaaggaaatatgtaaaccaaactttatgataaaaaaaatgcctggttatgatggttatccgagaatttgtgaactagttcgaagccaaatcactgacatcacaattcacggagcgtggtcaggtccagccagattggagttgtttgcccatgctttggcacccatgtctgaccttcctgtacttgaggttgtttcaaccagccatatcatcaccgacttgacattatcttcaccatccgttgtgcatgactatctaacaagtaagtga

SEQ ID NO: 101 - DNA, 747 bp, Artificial Sequence
  CdADC1 codon-optimized coding sequence
atgttggagaatgaggttatcaagcaaattactacaccaatttcagcccctgcattcccgaggggaccctacaagtttcacaacagagaatacctaaacatcatctaccgaacggatcttgaagctctcagaaagatcgtgccggaaccacttgaattggatggcccatatgttagatttgaaatgatggcaatgccagatacaacgggtttgggtagctacactgaatgcggacaggctattccagtgaccttcaacggcgtaagcggtgattacctacatatgatgtatcttgacaatgaaccggcaatagcggtcgggagggagtcctccgcttatccaaaaaaattggggaatcccaaacttttcgttgatagtgacactctcgtcgggacgttgaagtacggaaccttaccagttgctacagccacaatgggatataaacacgaacctttggatttgaaggaggcttatagtcaaatttcacggcctaactttatgttgaaagtcattcaaggttacgacggtaagccccgaatatgtgagttgatttgtgcagaaaataccgacattacaatccatggagcctggactggatctgcgcgtcttcaattgttttcgcatgcattggcgccattagccgacctccccgtgttagagatcatctcggcctctcacatactcacggacctaaccctaggcacaccaaagatagtatacgattatctttcaaagaaccagaaatga

SEQ ID NO: 102 - DNA, 741 bp, Clostridium saccharoperbutylacetonicum
atgctcgaaagtgaagtaagcaaacaaatcaccacgcccttagcggcccctgccttcccgagaggaccatatagatttcataatagggagtatcttaatatcatctatcggacggatcttgacgctttgcgaaagatcgtccccgagccacttgagttagatggtgcctacgtgagatttgagatgatggcgatgccggacaccactggtttgggatcttacactgaatgtgggcaggcaattccagtaaagtacaacggaattaagggtgactatctccatatgatgtaccttgacaacgaaccagcaattgctgtgggacgagaatcatctgcttacccaaagaaattgggctacccaaaactttttgttgattcagacacattggtcggtacattgaagtatggcaccctaccggttgcaacagcaacaatgggttataaacatgaacccttggatttgaaagaggcatacaaccagatagccagacccaacttcatgctaaagataatacaagggtacgatggaaagccacgtatctgcgagctaatatgtgctgaaaatacggacattactattcacggagcatggaccgggtcggctaggttgcaattgttcagccatgcgttggcccctttagcagacctccctgttttggaaattgtgtccgctagtcacatcctaactgatcttacgttaggcacaccaaaagtcgttcacgattatctctccttgaagtga

SEQ ID NO: 103 - protein, 350 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVI

SEQ ID NO: 104 - protein, 357 aa, Artificial Sequence
  CbADH1-P
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVILGRRAKL

SEQ ID NO: 105 - protein, 353 aa, Neurospora crassa
MPQFEIPEQQWAQVVEKKGGPVVFKQIPVQKPGPDEVLINVKYSGVCHTDLHAMKGDWPLATKMPLVGGHEGAGVVVAKGELVTEVEVGDHAGIKWLNGSCLACSFCMQADEPLCPHALLSGYTVDGSFQQYAIAKAAHVAKIPKGCDLETTAPVLCAGITVYKGLKESGVRPGQCVAIVGAGGGLGSMAIQYANAMGLHAIAIDGGEEKGKNCRELGAQAYVDFTTTKDLVADVKAATPDGLGPHAVILLAVSEKPFHQAVDYVRSRGTIICIGLPAGAKFQAPVFDTVIRMITIKGSYVGNRQDTQEALDFFARGLIKVPIKTVGLSKLQEVYDLMEEGKIVGRYVVDTSK

SEQ ID NO: 106 - protein, 359 aa, Artificial Sequence
  NcADH1-P
MPQFEIPEQQWAQVVEKKGGPVVFKQIPVQKPGPDEVLINVKYSGVCHTDLHAMKGDWPLATKMPLVGGHEGAGVVVAKGELVTEVEVGDHAGIKWLNGSCLACSFCMQADEPLCPHALLSGYTVDGSFQQYAIAKAAHVAKIPKGCDLETTAPVLCAGITVYKGLKESGVRPGQCVAIVGAGGGLGSMAIQYANAMGLHAIAIDGGEEKGKNCRELGAQAYVDFTTTKDLVADVKAATPDGLGPHAVILLAVSEKPFHQAVDYVRSRGTIICIGLPAGAKFQAPVFDTVIRMITIKGSYVGNRQDTQEALDFFARGLIKVPIKTVGLSKLQEVYDLMEEGKIVGRYVVDTSKGRRAKL

SEQ ID NO: 107 - protein, 336 aa, Candida parapsilosis
MSIPSSQYGFVFNKQSGLNLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 108 - protein, 342 aa, Artificial Sequence
  CpADH1-P
MSIPSSQYGFVFNKQSGLNLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNPGRRAKL

SEQ ID NO: 109 - protein, 336 aa, Candida viswanathii
MAIPATQTGFFFTKQEGLNYRTDIPVRKPQAGQLLLKVNAVGLCHSDLHVIDKELECGDNYVMGHEIAGTVAEVGPEVEGYKVGDRVACVGPNGCGVCKHCLTGNDNVCKTAFLDWFGLGSDGGYEEYLLVRRPRNLVKVPDNVSIEEAAAITDAVLTPYHAVKTAKVKPTSNVLVIGAGGLGGNGIQIVKAFGGKVTVVDKKDKARDQAKALGADEVYSEIPASIEPGTFDVCLDFVSVQATYDLCQKYCEPKGIIIPVGLGATKLTIDLADLDLREITVTGTFWGTANDLREAFDLVSQGKIKPIVSHAPLKELPNYMEKLKQGAYEGRVVFHP

SEQ ID NO: 110 - DNA, 1056 bp, Clostridium beijerinckii
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 111 - DNA, 1074 bp, Artificial Sequence
  CbADH1-P codon-optimized coding sequence
atgaagggatttgcaatgttgggaattaacaagttgggatggattgaaaaggaaagaccagttgcaggatcatacgatgcaattgttagaccattggcagtttcaccatgtacatcagatattcatacagtttttgaaggagcattgggagatagaaagaacatgattttgggacatgaagcagttggagaagttgttgaagttggatcagaagttaaggattttaagccaggagatagagttattgttccatgtacaacaccagattggagatcattggaagttcaagcaggatttcaacaacattcaaacggaatgttggcaggatggaagttttcaaactttaaggatggagtttttggagaatacttccatgttaacgatgcagatatgaacttggcaattttgccaaaggatatgccattggaaaacgcagttatgattacagatatgatgacaacaggatttcatggagcagaattggcagatattcaaatgggatcatcagttgttgttattggaattggagcagttggattgatgggaattgcaggagcaaagttgagaggagcaggaagaattattggagttggatcaagaccaatttgtgttgaagcagcaaagttttacggagcaacagatattttgaactacaagaacggacatattgttgatcaagttatgaagttgacaaacggaaagggagttgatcgagttattatggcaggaggaggatcagaaacattgtcacaagcagtttcaatggttaagccaggtggaattatttcaaacattaactaccatggatcaggagatgcattgttgattccaagagttgaatggggatgtggaatggcacataagacaattaagggaggattgtgtccaggaggaagattgagagcagaaatgttgagagatatggttgtttacaacagagttgatttgtcaaagttggttacacatgtttaccatggatttgatcatattgaagaagcattgttgttgatgaaggataagcccaaggatttgattaaggcagttgttattttgggtagacgagcaaagctttga

SEQ ID NO: 112 - DNA, 1062 bp, Neurospora crassa
atgcctcagttcgagattccagaacagcaatgggcccaggtggtcgagaagaagggcggtcccgtcgtcttcaagcagatccccgttcagaagcccggccccgatgaggtccttatcaacgtcaagtactctggcgtctgccataccgatctccatgccatgaagggtgactggcccctcgctaccaagatgcctctggtcggcggccacgaaggcgccggtgtcgtcgtagccaagggtgagctcgttactgaggttgaggttggcgaccacgccggtatcaagtggctcaacggctcctgcctggcctgctcattctgcatgcaggccgacgagcctctgtgccctcacgccctcctgtccggctacactgtcgacggttccttccagcaatacgctatcgccaaggccgcccacgtcgccaagatccccaaggggtgcgacctcgagaccaccgcgcccgtcctctgcgccggtatcaccgtctacaagggtctcaaggagtccggtgttcgtcccggccagtgcgtcgccatcgtcggcgccggcggtggtctcggcagcatggccattcagtacgccaacgccatgggcctgcacgccatcgccattgacggtggcgaggagaagggcaagaactgccgcgagctgggcgcccaggcctacgtcgacttcactactaccaaagaccttgtcgcggacgtcaaggctgccacccccgacggcctcggtccccacgccgtcatccttctcgccgtgtccgagaagccgttccaccaagccgtcgattacgtccgttcccgcggaaccatcatctgcatcggtctccccgctggcgccaagttccaggcccccgttttcgacaccgtcatccgcatgatcaccatcaagggctcgtatgtcggtaacaggcaggatacccaggaggcccttgacttctttgcccgcggcttgatcaaggttcccatcaagacggtcggcctcagcaaattgcaggaagtctacgacttgatggaggagggcaagattgtcggcagatatgtcgtcgataccagcaaatag

SEQ ID NO: 113 - DNA, 1080 bp, Artificial Sequence
  NcADH1-P codon-optimized coding sequence
atgccacaatttgaaattccagaacaacaatgggcacaagttgttgaaaaaaagggaggaccagttgtttttaagcaaattccagttcaaaagccaggaccagatgaggttttgattaacgttaagtactcaggagtttgtcatacagatttgcatgcaatgaagggagattggccattggcaacaaagatgccattggttggaggacatgaaggagcaggagttgttgttgcaaagggagaattggttacagaagttgaagttggagatcatgcaggaattaagtggttgaacggatcatgtttggcatgttcattttgtatgcaagcagatgaaccattgtgtccacatgcattgttgtcaggatacacagttgatggatcatttcaacaatacgcaattgcaaaggcagcacatgttgcaaagattccaaagggatgtgatttggaaacaaccgcaccagttttgtgtgcaggaattacagtttacaagggattgaaggaatcaggagttagaccaggacaatgtgttgcaattgttggagcaggaggaggattgggatcaatggcaattcaatacgcaaacgcaatgggattgcatgcaattgcaattgatggaggagaagaaaagggaaagaactgtagagaattgggagcacaagcatacgttgattttacaacaacaaaggatttggttgcagatgttaaggcagcaacaccagatggattgggaccacatgcagttattttgttggcagtttcagaaaagccatttcatcaagcagttgattacgttagatcaagaggaacaattatttgtattggattgccagcaggagcaaagtttcaagcaccagtttttgatacagttattcgaatgattacaattaagggatcatacgttggaaacagacaagatacacaagaagcattggatttttttgcacgaggattgattaaggttccaattaagacagttggattgtcaaagttgcaagaagtttacgatttgatggaagaaggaaagattgttggacgatacgttgttgatacatcaaagggtagacgagcaaagctttga

SEQ ID NO: 114 - DNA, 1011 bp, Candida parapsilosis
atgtcaattccatcaagccagtacggattcgtattcaataagcaatcaggacttaatttgagaaatgatttgcctgtccacaagcccaaagcgggtcaattgttgttgaaagttgatgctgttggattgtgtcattctgatttacatgtcatttacgaagggttggattgtggtgataattatgtcatgggacatgaaattgctggaactgttgctgctgtgggtgatgatgtcattaactacaaggttggtgatcgtgttgcctgtgtcggacccaatggatgtggtgggtgcaagtattgtcgtggtgccattgacaatgtatgtaaaaacgcatttggtgattggttcggattggggtacgatggtgggtatcaacagtacttgttggttactagaccacgtaacttgtctcgtatcccagataacgtatctgcagacgtggctgcggcttcaactgatgctgtattgacaccatatcacgcaatcaagatggctcaagtgtcaccaacttcgaatatcttgcttattggtgctggtggattgggtggaaatgcaattcaagttgccaaggcatttggtgcgaaagttactgttttggacaaaaaaaaggaggctcgtgaccaagcaaagaagttgggtgctgatgcagtttatgaaacattgccagaatccatttctcctggctctttttcagcatgttttgattttgtttcagtgcaagctacatttgatgtatgtcaaaagtatgttgaaccaaagggtgtaattatgcccgtgggactcggtgctcctaatttatcgtttaatttgggagatttggcattgagagaaattcgaatcttgggtagtttttggggaactactaatgatttggatgatgttttgaaattggttagtgaaggtaaagttaaacccgttgtgagaagtgccaaattgaaggaattgccagagtatattgaaaaattgagaaacaatgcttatgaaggtagagttgtttttaatccatag

SEQ ID NO: 115 - DNA, 1029 bp, Artificial Sequence
  CpADH1-P codon-optimized coding sequence
atgtcaattccatcatcacaatacggatttgtttttaacaagcaatcaggattgaacttgagaaacgatttgccagttcataagccaaaggcaggacaattgttgttgaaggttgatgcagttggattgtgtcatagtgatttgcatgttatctacgaaggtttggattgtggagataactacgttatgggacatgaaattgcaggaacagttgcagcagttggagatgatgttattaactacaaggttggagatagagttgcatgtgttggaccaaacggatgtggaggatgtaagtactgtagaggtgcaattgataacgtttgtaagaacgcatttggagattggtttggattgggatacgatggaggataccaacaatacttgttggttacaagaccaagaaacttgtcaagaattccagataacgtttcagcagatgttgcagcagcatcaacagatgcagttttgacaccataccatgcaattaagatggcacaagtttcaccaacatcaaacattttgttgattggagcaggaggattgggaggaaacgcaattcaagttgcaaaggcatttggagcaaaggttacagttttggataagaagaaggaagcaagagatcaagcaaagaagttgggagcagatgctgtttacgaaacattgccagaatcaatttcaccaggatcattttcagcatgttttgattttgtttcagttcaagcaacatttgatgtttgtcaaaagtacgttgaaccaaagggagttattatgccagttggattgggagcaccaaacttgagttttaacttgggtgatttggcattgagagaaattagaattttgggatcattttggggaacaacaaacgatttggatgatgttttgaagttggtttcagaaggaaaggttaagccagttgttcgatcagcaaagttgaaggaattgccagaatacattgaaaagttgagaaacaacgcatacgaaggaagagttgtttttaacccaggtagacgagcaaagctttga

SEQ ID NO: 116 - DNA, 1011 bp, Candida viswanathii
atggctatccccgctactcaaactggattcttcttcaccaaacaagaaggtttaaactacagaaccgacattcctgtccgcaagccacaagccggtcagttgttgttgaaggtcaatgccgttggtctctgccactcggacttgcacgtgattgacaaggagcttgaatgtggtgacaactatgtcatgggccacgaaattgccggtaccgttgctgaagttggtcccgaagttgaaggctacaaggttggcgaccgtgtcgcttgtgttggtcctaacgggtgcggtgtctgtaagcactgcttgactggtaacgacaatgtctgtaagactgctttcctcgactggttcgggttgggctccgatggtgggtacgaagagtacttgttggtgagaagaccaagaaacttggttaaggtcccggacaacgtctcgattgaggaggctgctgctatcactgatgctgtgttgactccttaccatgctgtcaagactgccaaggtcaagccaaccagtaacgttttggttattggtgctggtggattaggtggtaacggtatccagattgtcaaggcttttggcggtaaggttactgttgtcgataagaaggataaggcacgtgaccaagctaaggctttgggtgctgatgaagtctacagtgaaatcccagcaagtattgaaccgggtacttttgatgtctgtcttgattttgtttccgtgcaagccacctatgatctctgccaaaagtactgtgagccaaagggtatcattatcccagttgggttgggtgctaccaagctcaccattgatttggcagatttggatctccgtgaaatcacggttactggtaccttctggggaactgccaatgacttgagagaggcgtttgatttggttagtcaaggtaagatcaagccgattgtttcacatgccccattgaaggagttgccaaactatatggagaagttgaagcagggagcatatgaaggaagagttgtcttccacccataa

SEQ ID NO: 117 - DNA, 528 bp, Artificial Sequence
  POX4 promoter
gagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacatg

SEQ ID NO: 118 - DNA, 568 bp, Artificial Sequence
  PEX11 promoter
gaggatgaagaagacgaagacgaattggatgaagatgaagcgtatgagtattatgagtactgtcggacgttggaaggtggcagagttaagcccgagaaagcaaggaaggagtgggagatgatgagtgatgcggccaagaggatgtgaaggctgcgtatctgtttttgatagctggtggtagccgaatagaggaaggcaagcttgttcatattggatgatgatggtagatggtggctgccaaagtggttgtaaatagaaaaaagtgggtttgggtctgttgatagttagtggtggcggctgtctgtgattacgtcagcaagtagcacctcggcagttaaaacagcagcaacagaaaaaaaatgtgtgaaagtttgattcccccacagtctaccacacccagagttccatttatccataatatcacaagcaatagaaaaataaaaaattatcaacaaatcacaacgaaaagattctgcaaaattattttcacttcttcttttgacttcctcttcttcttgttaggttctttccatattttccccttaaacccatacacaacgcagccatg

SEQ ID NO: 119 - DNA, 504 bp, Artificial Sequence
  ETF1 promoter
gactgggagaacagaccaaggatgcccatacaccattaataacaagcacaccttgataaatctctagtgttgacaaattggtgatttgaacatgactgtagagagagagacaagtaaccactgatggattggtggtggcaaataaccactttaaataaagaccaactcacacacaaagacagcaggtcgtgttcctatttccaattttcacaaggagaagaataaaaatttttcaatgagattaactaaagaaacagacaggcagcccacaagaagaaagaagaagaagaggaagaggaagaagaagaatgaagaagaagaagaagagaaaaaaaatttttccctctgcgttgcgttgggcttgggttgccagcacccaccatatataactctcatcaaatacccagtagagaaaaatttttcctccctctttttctttctttcttctccttcttttgctactctttctgtttttcatcaaaaagatatatataatcaatcatg

SEQ ID NO: 120 - DNA, 502 bp, Artificial Sequence
  TDH3 promoter
accaaacgttactttttttttgcaatcggatggtatgggtctggggttcacctgttttgtaaagctacagaaggtggcatatttctctgatcaggtgttttttatttcggctgctgctgctcgtggtggtgtagtggtagtggtgtgtgtgtgtgtgtgtgcgtgcgtgtggaaggacgctttttgctctctgactcctcccaatcagaagttgctatagtggtgaaacaacaatggatgataatgccccgggcggtgcgtgtccgacacaaaccactacattttttagctgggagcctactgccactacgacccacccacccatggtcaacaaaaaaattctgacaaattataaaataacccttgaattcccccttggaaaaatttttggtatttctctctctcttttcctttccctcttctttttctctccatcaatcaattgacgttcagtaactcaattaattacatcacatccctcaattaaagaatttaaacaatg

SEQ ID NO: 121 - DNA, 200 bp, Artificial Sequence
  POX4 terminator
gaatagaagagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgacctctcatgatttcaac

SEQ ID NO: 122 - DNA, 200 bp, Artificial Sequence
  ETF1 terminator
actaattaatgaataattaatgagtattgttttatatatatcttgaagtaataaataactaaaataaaaaaaaagagagagagagagtaagttgtagctttcattaacattatggttgtgtattaaaagaatagtagaattatttatatcagtacagcttggaaacctttgccactaacaacacccatccaatcaccctg

SEQ ID NO: 123 - DNA, 2313 bp, Artificial Sequence
  URA3::(PPOX4-TPOX4)
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagactatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagatcctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtcggagtgagacgggagagagaccaatagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacatgagaagagcccgggtctagatgtgtgctcttccgagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgatggtctctcgctcgtctccgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaa

SEQ ID NO: 124 - DNA, 1085 bp, Artificial Sequence
  URA3-A
tacagctcttccatgatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggtactggatcgcaagcaacgagaacgccgccgatgctccggcaagcaacacaaacgaggacttcaagatcgatctggtttggattgttggagaatttcaagaatctcaagatttactctaacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaataaaaaaagtgaaataaaaaatcaaacccaaagatcaacctccccatcatcatcgtcatcaaacccccagctcaattcgcaatggttagcacaaaaacatacacagaaagggcatcagcacacccctccaaggttgcccaacgtttattccgcttaatggagtccaaaaagaccaacctctgcgcctcgatcgacgtgaccacaaccgccgagttcctttcgctcagagaccataggtctcagatcgagggaagagctgtaca

SEQ ID NO: 125 - DNA, 1085 bp, Artificial Sequence
  URA3-B
tacagctcttccatggatctgagacctatggtctctgagcgaaaggaactcggcggttgtggtcacgtcgatcgaggcgcagaggttggtctttttggactccattaagcggaataaacgttgggcaaccttggaggggtgtgctgatgccctttctgtgtatgtttttgtgctaaccattgcgaattgagctgggggtttgatgacgatgatgatggggaggttgatctttgggtttgattttttatttcactttttttatttcttgggagttggggaatggatgagatatctcacgtgactagtatggcaatttattgtataaatctgtattccttactttgtttgttatataatacaaaatacttatttaccttcttcttcttgtttcacctcttcgtttgcttcttcctgtttagcttctggttgaccactttccataaactctttcttcaattgtggataaatctcatccattttatcacatgacgccttgtatatctgtgcattctcagtctggtccaagaacttgatgttctgtaacaccaagatcatgttcttgatcaattcaatacaattctcgttgtacccgtcgttagagtaaatcttgagattcttgaaattctccaacaatccaaaccagatcgatcttgaagtcctcgtttgtgttgcttgccggagcatcggcggcgttctcgttgcttgcgatccagtacccgatttcaaaagtgcagaagatccatttatcgaggttggaagcgttggatgtgtataatcattacaacgaggcgaagctcaagggtcagacgaagccgttcttcaaggtgtattacaactcgtacgtggataccttcaagcaagtgatgcggatgaagcaaagggcgaatgtgacgtggtttgaggcggcgtactctgggtttgtcaggaacgcactaaccaccatccctgctacatccattgcgttattggttttcgaaatcatgagaactaaactagcagacaacttcgaagactacaatgacgtcctagaagcttagtgtatgtatatagacctatttattacaatatgagggaagagctgtaca

SEQ ID NO: 126 - DNA, 1106 bp, Artificial Sequence
  URA3-C
acaggtctcagctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgaaacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagggaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggtactggatcgcaagcaacgagaacgccgccgatgctccggcaagcaacacaaacgaggacttcaagatcagagaccata

SEQ ID NO: 127 - DNA, 594 bp, Artificial Sequence
  GRE3-A
ctcgagttaattaaccacattccactccattgggcattttcgacctttggggttcatctgattgcaaaatgaagaaaaagaaacgctggcattgtttcggaataactttccccagtgtttgcagagaaatttttgccaattactttacctcctccacccgtttcaatttattatttaaacacaacaatagcgtcgtttcttgttccaactgcttaccattttcctgacacagtgtttagttggggaagcaattcaacattcaaaaaagattgcttgaatagtgtggctcgcaacgactttctctccgccctgcgaaaaactcccgtgctccctccccagaatcttactccgatgtttagacttttctcaacaactataaagggatcacgaatcctcagacattcttgccattcctttcctagaccattatcaacttaccaaacaacaaacatgtctactgctactgcttccccaactatcaaattgaactccggctacgaaatgccattggttgggagtgagacgggagagagaccaatggtctctcgctcgtctcacgcttaatgagaagagcatagctcttccgctggatcc

SEQ ID NO: 128 - DNA, 536 bp, Artificial Sequence
  GRE3-B
acagctcttccgaggcagatcgaacaccacccatacttgcaacagccaaagttgatcgagtacgtccagaagcaaggcatcgccatcaccgggtactcctccttcggtccacagtccttcttggagttggagtccaagagagccttggacacccctaccttgttccagcacgagactatcaagtccattgccgacaagcacggcaagtcgccagcccaggtcttgttgagatgggctacccagagaggcatcgctgtcattccaaagtccaactccccagacagattggctcagaacttgtctgtcgtcgactttgacttgacccaggaggacttggacgagattgccaagttggacattggcttgagattcaacgacccatgggactgggacaacattccaatctttgtttaaaggaacactccgtagttattatgtacatgcgattaatagatttatattgcgtctaagacggcttgtctgccgagaactagtaacttcaatctacttacaattaattaagctagaagagcaca

SEQ ID NO: 129 - DNA, 537 bp, Artificial Sequence
  GRE3-C
acagctcttccgaggctgctggaaagtcaccaacgctaccgctgccgaccagatctacaacgccatcaagaccggctacagattgttcgacggtgccgaggactacggcaacgagaaggaagtcggtgaaggtatcaacagagccatcaaggacggcttggtcaagagagaagagttgttcattacctccaagttatggaacaacttccacgacccaaagaacgtcgaggtcgctttgaacaagaccttgagcgacttgaacttggactacgtcgacttgttcttgatccacttcccaattgctttcaagtttgtcccaattgaagaaaaatacccacctggcttctactgcggagaaggcgacaagttccactacgaagacgtcccattgttggacacctggaaggctttggagaagttggttgacgccggcaagatcaagtccatcggtatctccaacttcaccggtgcattgatctacgacttgatcaggggcgccaccatcaagcccgccgttaattaagctagaagagcaca

SEQ ID NO: 130 - DNA, 594 bp, Artificial Sequence
  CAT2a
ctcgagttaattaacaccacacatacatcccccaacaatccaacatcccgaaggagtcaaaaaacaagagagatagagatgccggaaagggggttgttctttttcggagttgctggtccttgaagagaaagtattttctgagtggtaaccgcataccgtgggtattggacaatgagctcatcaagatccacgaggttcctcggttgaacgcttcattttttctttggattcctggttttttttgggccgtggtgcacacaccccccgtacattttcagcgccactgcttgacccccacaaaatttatcataagaaaataattggcactgattctcaacctcaacgaatatataaaatcaacaaaatttttcaactgagaattgtttttttcttttaaaactctcttttcttgttctttgttttcatataaaacgtttttaattttccctccttttttttattccatcatagagtttcttttcttcaattgagtaaattgtaggagagaggagatggagtgagacgggagagagaccaatggtctctcgctcgtctcacgcttaatgagaagagcatagctcttccgctggatcc

SEQ ID NO: 131 - DNA, 536 bp, Artificial Sequence
  CAT2b
acagctcttccgagcgttgcctattgtctgctgctgccgatggtaagggtgttgaccgtcacttgtttggtttgaagcaaatgattcaaccaggcgaaccaatccctgaaatcttcactgacccaatcttcagctattctcaaacctggtacatttcttcttcccaagtccatctgaattcttccaatcttggggttggtcgcaagtcattgatgacggtttcggtttggcttacttgatcaacaacgactcgatccacgttcacatttcttgtaagagaggcaacggcttgcaatctgaccacttgaaatggtacttggttgaaagtgctaatgaaatgaaggatgtttgactaagggattattgactgatgctaagcctaagttgtaagtccgcgggagaacaaacagttattatccgccttacggctcttttttttggctgcaacatttagcctcagaataaaaagcaacagaaccctggtccttttttagctcatttcatttcatttcagttaattaagctagaagagcaca

SEQ ID NO: 132 - DNA, 536 bp, Artificial Sequence
  CAT2c
acagctcttccgagactccagaatggacgctaccccaaccgtgcaattgaacaacaccatctacaagcaaatcttggaaaccgatccaaacgacttgattgttgaaattggctcttttgctccaagattcggcaatgctgaaatcttgcctttcgacattaacccaaccaccagagccaacatcaaagacgctattgccaagtttgacgccaccattgctgcccacgacgaagaaatcttccaacactatggttacggtaagggcttgatcaagaagttcaacgtctccccagatgcctacgtgcaattgttgatgcaattggcatactttaagtacaccggcaagatcagaccaacttatgaatccgccgccaccagaaagttcttgaagggtagaaccgaaaccggtagaactgtctccaacgaatccaagaagtttgttgagacttggtccgatccaaaggccagcagcgccgacaaggttgccactttccaagctgctgctaagcaacacttaattaagctagaagagcaca

SEQ ID NO: 133 - DNA, 575 bp, Artificial Sequence
  5′ADH5a
ctcgagttaattaacgacttattagggagcaaaattgcccgcacattgtctaggctttgtggcacgtttttttcccacccccacattaaacaacattcgatttttccaatgcactttttattatttgcaccattgtctcccactctgttatcagctgtgtatatgtgcagtttgctaaagctggcaattgtctacgtaaatattactttgggaaactgggagttgggcccatggcggcataaaacagcgtgattgcacttgccactcccacaagtcttgtccccacgcctcgttggagcaaccctattcccccgaatcgctcctgtatatttaaaatactccctatcccccctccttttccgagcttattttaagttgtttattgtctaacaacaaagggaaagcaagaattgccttggtataaaaaataggagactccgatagcccccagaacatataaataaaattgcaaaccttctttctagtatataaaatggagtgagacgggagagagaccaatggtctctcgctcgtctcacgcttaatgagaagagcatagctcttccgctggatcc

SEQ ID NO: 134 - DNA, 536 bp, Artificial Sequence
  3′ADH5a
acagctcttccgagtggagcccccttatatatatatattctataccgtaggaactagcaaaagaaaaagagaatagcatagtgaacatactgcaacaagaaggaatacttccgttttcagtaatttttgcgtctgccgcacaatatgtccgagtttgtaaaaggtgcataaactgagcagaaaaacaaaaaaagaaacactggccacacagagaaaattgagtaacttcctaagctcgaattatgcagctcgtgcgtcaacctatgtgcaggaaagaaaaaatccaaaaaaatcgaaaatgcgactttcgattttgaataaaccaaaaagaaaaatgtcgcacttttttctcgctctcgctctctcgacccaaatcacaacaaatcctcgcgcgcagtatttcgacgaaaccacaacaaataaaaaaaacaaattctacaccacttctttttcttcaccagtcaacaaaaaacaacaaattatacaccatttcaacgatttttgctcttataaattaattaagctagaagagcaca

SEQ ID NO: 135 - DNA, 595 bp, Artificial Sequence
  5′ADH5b
ctcgagttaattaacttgcgacttattaggaagcaaaattgcctgcacactgcttaggctttgtgacactttttttcttagtttagagctcttccctcccacccccacattaaacaacattcgatttttccaatgcacctttattatttgcgccattgtctcccactctgttatcagctgtgtatatgtgcagtttgctaaagctggtgattgtctacgtaaatattactttgggaaactgggagttgggcccatggcggcataaaacagcgtgattgcacttgtcatccccacaagtcttgtcctcacgcctcattggagcaatcatattcccccgaatcgctcctgtatatttaaaatactccctatccccccttttccgggcttattttaagttgtttattgtctaacaacaaaggaaaagcaagaattgccttggtataaaaaaaaggagtctccgatagccccaaaacacataaataaaattgcaaacctttttttttagtatataaaatggagtgagacgggagagagaccaatggtctctcgctcgtctcacgcttaatgagaagagcatagctcttccgctggatcc

SEQ ID NO: 136 - DNA, 538 bp, Artificial Sequence
  3′ADH5b
acagctcttccgagactagtaacgttttgattattggtgccggcgggttgggtggtaacggtatccagactgtcaaggcttttggcggtaaggttactgttgtcgataagaaggagaaggcacgtgaccaagctaaggttttgggtgctgatgaagtctacagtgaaattccaacaagtgttgagccaggtacttttgatgtctgtcttgattttgtttccgtgcaagccacctttgatctctgccaaaagtactgtgagccaaagggtatcattatcccagttgggttgggtgctaccaagctcaccattgacttggctgatttggatctccgcgaaatcacggttactggtaccttctggggaacagccaacgacttgagagaggcgtttgatttggttagtcagggtaagatcaagccgattgtttctcatgccccattgaaggagttgccaaactatatggagaagttgaagcagggagcttatgaaggaagagttgtcttccacccataatttaattaagctagaagagcaca

SEQ ID NO: 137 - DNA, 11 bp, Artificial Sequence
  5′ Addition
acagctcttcc

SEQ ID NO: 138 - DNA, 13 bp, Artificial Sequence
  5′ Addition
atggtctcaggag

SEQ ID NO: 139 - DNA, 13 bp, Artificial Sequence
  5′ Addition
atggtctcagtca

SEQ ID NO: 140 - DNA, 14 bp, Artificial Sequence
  5′ Addition
agagctcttccgag

SEQ ID NO: 141 - DNA, 20 bp, Artificial Sequence
  3′ Addition
taagagggaagagctgtaca

SEQ ID NO: 142 - DNA, 32 bp, Artificial Sequence
  3′ Addition
agaagagcgctcttccgctgtcatgagaccat

SEQ ID NO: 143 - DNA, 32 bp, Artificial Sequence
  3′ Addition
agaagagcgctcttccgctcgcttgagaccat

SEQ ID NO: 144 - DNA, 86 bp, Artificial Sequence
  3′ Addition
gctagaagagcagatcgacccacagggaggaagtaaactgctattcgtcgccgatggtggtaactaattatgttccttgccactat

SEQ ID NO: 145 - DNA, 23 bp, Artificial Sequence
  Oligo o1
ggaagtgccattccgcctgacct

SEQ ID NO: 146 - DNA, 76 bp, Artificial Sequence
  Oligo o3
tgtacagctcttccctcttatcaaagctttgctcgtctacccttaactgacaagtaatcatgaacaacctttggtg

SEQ ID NO: 147 - DNA, 27 bp, Artificial Sequence
  Oligo o8
ccttcggagtttcgtatccttatcacc

SEQ ID NO: 148 - DNA, 35 bp, Artificial Sequence
  Oligo o9
cttttattgtatcgttgtttctgtgtgcataacac

SEQ ID NO: 149 - DNA, 23 bp, Artificial Sequence
  Oligo o10
ggatcgcaagcaacgagaacgcc

SEQ ID NO: 150 - DNA, 23 bp, Artificial Sequence
  Oligo o11
ggcgttctcgttgcttgcgatcc

SEQ ID NO: 151 - DNA, 28 bp, Artificial Sequence
  Oligo o12
caagagctgttttcccatctatcgttgg

SEQ ID NO: 152 - DNA, 34 bp, Artificial Sequence
  Oligo o13
gtataattcttttctgacatccaagtcacacttc

SEQ ID NO: 153 - DNA, 37 bp, Artificial Sequence
  Oligo o14
gttgtcacaacaagtattaaagaattcaaccaaatcc

SEQ ID NO: 154 - DNA, 26 bp, Artificial Sequence
  Oligo o15
ctgggtccaagctttcatccaaaacc

SEQ ID NO: 155 - DNA, 34 bp, Artificial Sequence
  Oligo o16
gatttgaaaacttgagatccttctttaaggatgg

SEQ ID NO: 156 - DNA, 32 bp, Artificial Sequence
  Oligo o17
cccttgtaaaatgtgtttcctgcttcatcaac

SEQ ID NO: 157 - DNA, 28 bp, Artificial Sequence
  Oligo o18
cagaaaacggaattgttggaatgggagc

SEQ ID NO: 158 - DNA, 33 bp, Artificial Sequence
  Oligo o19
cctcaattcgtttgaaatcaacaaatctgctgc

SEQ ID NO: 159 - DNA, 33 bp, Artificial Sequence
  Oligo o20
ggatagagcatacgttagatttgaaatgatggc

SEQ ID NO: 160 - DNA, 27 bp, Artificial Sequence
  Oligo o21
ccaaaactggcaaatcagccaatggtg

SEQ ID NO: 161 - DNA, 35 bp, Artificial Sequence
  Oligo o22
cacaggaattggaaagttgatttcaaacaaccaag

SEQ ID NO: 162 - DNA, 26 bp, Artificial Sequence
  Oligo o23
caaagctttgctcgtctacctgctgg

SEQ ID NO: 163 - DNA, 24 bp, Artificial Sequence
  Oligo o24
gctcgtctaccaactgccattggc

SEQ ID NO: 164 - DNA, 28 bp, Artificial Sequence
  Oligo o25
caaagctttgctcgtctacccttaactg

SEQ ID NO: 165 - DNA, 53 bp, Artificial Sequence
  Oligo o34
tgtacagctcttccctcttacaaaataacaactgccttaatcaaatccttggg

SEQ ID NO: 166 - DNA, 54 bp, Artificial Sequence
  Oligo o35
tgtacagctcttccctcttactttgatgtatcaacaacgtatcgtccaacaatc

SEQ ID NO: 167 - DNA, 56 bp, Artificial Sequence
  Oligo o36
tgtacagctcttccctcttaatctgcatcaatcttttcaataacaactgctgatgc

SEQ ID NO: 168 - DNA, 50 bp, Artificial Sequence
  Oligo o37
tgtacagctcttccctcttatgggttaaaaacaactcttccttcgtatgc

SEQ ID NO: 169 - DNA, 29 bp, Artificial Sequence
  Oligo o38
gaagcagttggagaagttgttgaagttgg

SEQ ID NO: 170 - DNA, 27 bp, Artificial Sequence
  Oligo o39
cgatcaactccctttccgtttgtcaac

SEQ ID NO: 171 - DNA, 30 bp, Artificial Sequence
  Oligo o40
ctcaggagtttgtcatacagatttgcatgc

SEQ ID NO: 172 - DNA, 31 bp, Artificial Sequence
  Oligo o41
ccttttcttctcctccatcaattgcaattgc

SEQ ID NO: 173 - DNA, 26 bp, Artificial Sequence
  Oligo o42
gcaagcaaacgttggacaagcaccag

SEQ ID NO: 174 - DNA, 38 bp, Artificial Sequence
  Oligo o43
gacttcaatctttcttcgttaaactttccaatttcttc

SEQ ID NO: 175 - DNA, 31 bp, Artificial Sequence
  Oligo o44
gagaaacgatttgccagttcataagccaaag

SEQ ID NO: 176 - DNA, 27 bp, Artificial Sequence
  Oligo o45
cttgaattgcgtttcctcccaatcctc

SEQ ID NO: 177 - DNA, 45 bp, Artificial Sequence
  Oligo o56
atggtctcagtcaagtagagcgatcttccgctcgcttgagaccat

SEQ ID NO: 178 - DNA, 45 bp, Artificial Sequence
  Oligo o57
atggtctcaagcgagcggaagatcgctctacttgactgagaccat

SEQ ID NO: 179 - DNA, 28 bp, Artificial Sequence
  Oligo o106
gatctcgttgggaaggaattatggagtg

SEQ ID NO: 180 - DNA, 25 bp, Artificial Sequence
  Oligo o107
cactcgacgtctgagcacgttatgg

SEQ ID NO: 181 - DNA, 25 bp, Artificial Sequence
  Oligo oF1
caggagacaagatcattcgctgcgg

SEQ ID NO: 182 - DNA, 19 bp, Artificial Sequence
  Oligo oF2
gctcggcgtgttgtgggcg

SEQ ID NO: 183 - DNA, 23 bp, Artificial Sequence
  Oligo oF3
gggaaatattgtgcggcagccgc

SEQ ID NO: 184 - DNA, 25 bp, Artificial Sequence
  Oligo oF4
ctacagaaccgacattcctgtccgc

SEQ ID NO: 185 - DNA, 28 bp, Artificial Sequence
  Oligo oF5
gggaccttaaccaagtttcttggtcttc

SEQ ID NO: 186 - protein, 3 aa, Artificial Sequence
  C-terminal signal
SKL

SEQ ID NO: 187 - protein, 6 aa, Artificial Sequence
  C-terminal signal
GRRAKL

(19) United States
(12) Patent Application Publication
(10) Pub. No.: US 2024/0218383 A1
(43) Pub. Date: Jul. 4, 2024

(54) RECOMBINANT YEASTS FOR PRODUCING ACETONE AND/OR ISOPROPANOL FROM FATTY ACID FEEDSTOCKS

(71) Applicant: CIRCULAR INDUSTRIES HOLDING PTE LTD., Singapore (SG)

(72) Inventor: José Miguel Laplaza, Milwaukee, WI (US)

(73) Assignee: CIRCULAR INDUSTRIES HOLDING PTE LTD., Singapore (SG)

(21) Appl. No.: 18/557,456

(22) PCT Filed: May 11, 2022
(86) PCT No.: PCT/SG2022/050305

§ 371 Date: Oct. 26, 2023

Related U.S. Application Data
(60) Provisional application No. 63/187,462, filed on May 12, 2021.

(51) Int. Cl. C12N 15/81; C12N 9/04; C12N 9/10; C12N 9/16; C12N 9/88; C12P 7/04; C12P 7/28; C12R 1/72

(52) U.S. Cl. CPC C12N 15/815; C12N 9/0006; C12N 9/1029; C12N 9/16; C12N 9/88; C12P 7/04; C12P 7/28; C12R 2001/72; C12Y 101/0108; C12Y 203/01009; C12Y 301/02001; C12Y 301/02011; C12Y 401/01004

(57) ABSTRACT

Recombinant yeasts bioengineered to overexpress genes for utilization of fatty acids to produce acetone and isopropanol, and methods of use thereof. The yeasts are modified to express, constitutively express, or overexpress an acetyl-CoA thioesterase, an acetyl-CoA C-acetyltransferase, an acetoacetyl-CoA transferase, an acetoacetyl-CoA thioesterase, an acetoacetate decarboxylase, an isopropanol dehydrogenase, or any combination thereof. The methods include cultivating the recombinant yeasts to convert any fatty acid-containing feedstocks into acetone and/or isopropanol.

### FIELD OF THE INVENTION

The present invention relates to strains of recombinant yeasts suitable for producing acetone and/or isopropanol from fatty acid feedstocks, and methods of using the recombinant yeasts for producing acetone and/or isopropanol from fatty acid feedstocks.

### BACKGROUND

Isopropanol and acetone are essential commodity chemicals in the global chemical industry. They are used as solvents and as chemical intermediates. For example, isopropanol is used to make isopropyl acetate. Acetone is an intermediate to make methyl methacrylate, bisphenol A, methyl isobutyl alcohol, and methyl isobutyl ketone. Global production of acetone and isopropanol were 7.3 and 2.2 million tons in 2020.

Most of the isopropanol and acetone in the market are made from non-renewable sources. Isopropanol is mostly produced by the indirect and direct hydration of propene, although it can also be made by acetone's hydrogenation. Acetone is commonly made by the cumene process. This process synthesizes acetone from benzene and propylene.

Production of isopropanol and acetone from fermentation has been of interest for many years. Acetone can be made from sugars by the ABE (acetone butanol ethanol) fermentation of sugars using some Clostridium clade members, such as Clostridium beijerinckii and Clostridium acetobutylicum. Acetone is produced at half the amount of butanol during the fermentation. Isopropanol can also be made from sugar with a similar process to the ABE fermentation, where the acetone is replaced by isopropanol. This process is referred to as IBE fermentation and is performed with different wild-type Clostridium strains or metabolically engineered ABE-producing Clostridium strains.

Researchers have also engineered Escherichia coli and Candida utilis to produce acetone and isopropanol from sugar. Recent work in this area demonstrates a commercial interest in a fermentation-based renewable process to create these two solvents. One approach to competing commercially with the non-renewable source processes is to use lower value cellulosic sugar or syngas as a feedstock. To our knowledge, there is no current commercial process for isopropanol or acetone using either cellulosic sugar or syngas as a feedstock.

The biosynthesis of the isopropanol and acetone in Clostridium starts with acetyl-CoA (see FIG. 1). When grown in dextrose, two molecules of acetyl-CoA are produced per molecule of sugar by glycolysis. Two acetyl-CoA are used to create one acetoacetyl-CoA and a free molecule of CoA. An acetyl-CoA C-acetyltransferase catalyzes this reaction. The acetoacetyl COA is converted to acetoacetate by transferring the CoA to a butyrate molecule catalyzed by a butyrate-acetoacetate CoA-transferase. Next, an acetoacetate decarboxylase decarboxylates the acetoacetate to produce acetone. Acetone is then reduced to isopropanol using an isopropanol dehydrogenase. NADPH provides the hydrogen for the reaction.

Tamakawa et al. 2013 engineered Candida utilis, a yeast, to produce isopropanol from dextrose. The strain expressed the genes that encode the aceto-acetyl-CoA transferase (ctfA and ctfB) from Clostridium acetobutylicum, and the acetoacetate decarboxylase (adc) and the isopropanol dehydrogenase (sadh) genes from Clostridium beijerinckii. In addition, they overexpressed the native aceto acetyl-CoA transferase and the acetyl-CoA synthase genes to increase production.

The maximum theoretical yields of acetone and isopropanol on a g/g basis from sugar is 0.48 and 0.44 (Dellomarco et al. 2010). In contrast, the theoretical yields from fatty acids is 1.30 and 1.20, respectively. Although vegetable oil prices are higher than sugar, the economics still favor vegetable oil as a feedstock. This advantage is even more pronounced when you consider that cheap sugars such as cellulosic sugars are still expensive to use due to the processing required to make them available and compatible with fermentation. In contrast, low-value oil streams such as used frying oil and fatty acid distillates require minimum processing to be used as a feedstock in fermentation. Besides having the right feedstock for fermentation, an organism that can consume that feedstock is essential.

Yeasts that produce acetone and isopropanol from fatty acid-containing feedstocks are needed.

### SUMMARY OF THE INVENTION

The invention addresses the aforementioned needs of acetone and isopropanol production by providing recombinant yeasts. The recombinant yeast can be used, for example, in methods for converting fatty acids, fatty acid esters, fatty acid alcohols, vegetable oil, and other fatty acid-containing feedstocks into acetone and isopropanol.

One aspect of the invention is directed to recombinant yeasts. The recombinant yeasts are modified to express, constitutively express, or overexpress any one or more of an acetyl-CoA thioesterase, an acetyl-CoA C-acetyltransferase, an acetoacetyl-CoA transferase, an acetoacetyl-CoA thioesterase, an acetoacetate decarboxylase, and an isopropanol dehydrogenase. In some cases, the recombinant yeasts are modified to express any one or more of an acetoacetyl-CoA transferase and an acetoacetate decarboxylase. In some cases, the recombinant yeasts are modified to express any one or more of an acetoacetyl-CoA transferase, an acetoacetate decarboxylase, and an isopropanol dehydrogenase. The recombinant yeasts of the invention can be used for converting fatty acids into acetone and isopropanol to be used as essential commodities chemicals.

Also provided herein are methods of producing a product. Some versions comprise contacting the substrate comprising a first organic with the yeast of the invention, wherein the yeast consumes the first organic and produces a second organic. In some versions, the first organic comprises fatty acid. In some versions, the second organic comprises one or more of acetone and isopropanol. The method may find use for converting fatty acids, fatty acid esters, fatty acid alcohols, vegetable oil, and any other fatty acid-containing feedstocks into acetone and isopropanol.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Biosynthesis of acetone and isopropanol in Clostridium.

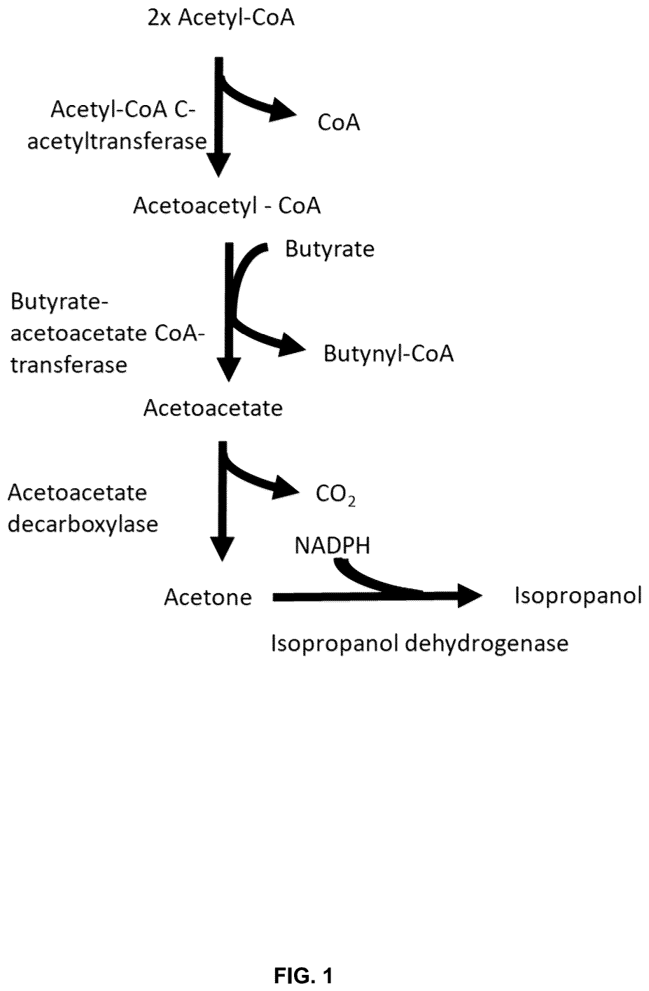

FIG. 2. Acetone pathway in a recombinant yeast expressing an acetoacetyl-CoA transferase (E3 activity) and an acetoacetate decarboxylase (E5 activity).

FIG. 3. Acetone pathway in a recombinant yeast expressing an acetyl-CoA thioesterase (E1 activity), an acetoacetyl-CoA transferase (E3 activity), and an acetoacetate decarboxylase (E5 activity).

FIG. 4. Acetone pathway in a recombinant yeast expressing an acetoacetyl-CoA thioesterase (E4 activity) and an acetoacetate decarboxylase (E5 activity).

FIG. 5. Acetone pathway in a recombinant yeast expressing an acetoacetyl-CoA transferase (E3 activity) and an acetoacetate decarboxylase (E5 activity) in the peroxisome.

FIG. 6. Acetone pathway in a recombinant yeast expressing an acetyl-CoA thioesterase (E1 activity), an acetoacetyl-CoA transferase (E3 activity), and an acetoacetate decarboxylase (E5 activity) in the peroxisome.

FIG. 7. Acetone pathway in a recombinant yeast expressing an acetoacetyl-CoA thioesterase (E4 activity) and an acetoacetate decarboxylase (E5 activity) in the peroxisome.

FIG. 8. Acetone and isopropanol pathway in a recombinant yeast expressing an acetoacetyl-CoA transferase (E3 activity), an acetoacetate decarboxylase (E5 activity), and an isopropanol dehydrogenase (E6 activity).

FIG. 9. Acetone and isopropanol pathway in a recombinant yeast expressing an acetyl-CoA thioesterase (E1 activity), an acetoacetyl-CoA transferase (E3 activity), an acetoacetate decarboxylase (E5 activity), and an isopropanol dehydrogenase (E6 activity).

FIG. 10. Acetone and isopropanol pathway in a recombinant yeast expressing an acetoacetyl-CoA thioesterase (E4 activity), an acetoacetate decarboxylase (E5 activity), and an isopropanol dehydrogenase (E6 activity).

FIG. 11. Acetone and isopropanol pathway in a recombinant yeast expressing an acetoacetyl-CoA transferase (E3 activity), an acetoacetate decarboxylase (E5 activity), and an isopropanol dehydrogenase (E6 activity) in the peroxisome.

FIG. 12. Acetone and isopropanol pathway in a recombinant yeast expressing an acetyl-CoA thioesterase (E1 activity), an acetoacetyl-CoA transferase (E3 activity), an acetoacetate decarboxylase (E5 activity), and an isopropanol dehydrogenase (E6 activity) in the peroxisome.

FIG. 13. Acetone and isopropanol pathway in a recombinant yeast expressing an acetoacetyl-CoA thioesterase (E4 activity), an acetoacetate decarboxylase (E5 activity), and an isopropanol dehydrogenase (E6 activity) in the peroxisome.

FIG. 14. Acetone and isopropanol pathway in a recombinant yeast expressing an acetyl-CoA thioesterase (E1 activity) in the peroxisome and expressing an acetoacetyl-CoA transferase (E3 activity), an acetoacetate decarboxylase (E5 activity), and an isopropanol dehydrogenase (E6 activity) in the cytoplasm.

FIG. 15. Acetone and isopropanol pathway in a recombinant yeast expressing an acetyl-CoA thioesterase (E1 activity) in the peroxisome and expressing an acetoacetyl-CoA thioesterase (E4 activity), an acetoacetate decarboxylase (E5 activity), and an isopropanol dehydrogenase (E6 activity) in the cytoplasm.

FIG. 16. Acetone and isopropanol pathway in a recombinant yeast expressing an acetoacetyl-CoA thioesterase (E4 activity) in the peroxisome and expressing an acetoacetate decarboxylase (E5 activity) and an isopropanol dehydrogenase (E6 activity) in the cytoplasm.

FIG. 17. Acetone and isopropanol pathway in a recombinant yeast expressing an acetoacetyl-CoA thioesterase (E4 activity) and an acetoacetate decarboxylase (E5 activity) in the peroxisome and expressing an isopropanol dehydrogenase (E6 activity) in the cytoplasm.

FIG. 18. Acetone and isopropanol pathway in a recombinant yeast expressing an acetoacetyl-CoA thioesterase (E4 activity) and an isopropanol dehydrogenase (E6 activity) in the peroxisome and expressing an acetoacetate decarboxylase (E5 activity) in the cytoplasm.

FIG. 19A. GC analysis of acetone standard.

FIG. 19B. GC analysis of acetone from the exemplary YU5 yeast.

FIG. 19C. GC analysis of acetone from the exemplary YU6 yeast.

### DETAILED DESCRIPTION OF THE INVENTION

An aspect of the invention encompasses recombinant yeasts. The recombinant yeasts are preferably engineered to have enhanced activities to produce acetone and isopropanol with respect to their native counterparts.

The recombinant yeasts are preferably derived from yeasts that consume fatty acids, such as Candida viswanathii, Candida tropicalis, Candida utilis, Yarrowia lipolytica, and Arxula adeninivorans. Candida viswanathii is a particularly preferred yeast in this regard. Candida viswanathii consumes alkanes and fatty acids at a remarkably high rate. There are well-established protocols and tools for its genetic manipulation, and Candida viswanathii has been engineered to produce diacids, cannabinoids, 3-hydroxy propionic acid, and carotenoids. It has also been used commercially to produce diacids from fatty acids and alkanes, demonstrating its ability to scale-up and function in an industrial setting. Genetic manipulations in Candida viswanathii are made as described in various patents and patent applications (CA3069708A1, U.S. Pat. No. 9,938,544B2, U.S. Pat. No. 9,957,512B2), or using other well established molecular techniques.

The recombinant yeasts of the invention comprise one or more recombinant nucleic acids configured to express one or more enzymes. The one or more recombinant nucleic acids are preferably configured to constitutively express or to overexpress the one or more enzymes. The one or more recombinant nucleic acids preferably comprise one or more recombinant genes configured to constitutively express or to overexpress the one or more enzymes. If a cell endogenously expresses a particular enzyme, the nucleic acid expressing that enzyme may be modified to exchange or optimize promoters, exchange or optimize enhancers, or exchange or optimize any other genetic element that results in increased or constitutive expression of the enzymes. Alternatively or additionally, one or more additional copies of a gene or coding sequence thereof may be introduced to the cell for enhanced expression of the enzymes. If a cell does not endogenously express a particular enzyme, one or more copies of a recombinant nucleic acid configured to express that enzyme may be introduced to the cell for expression of the enzyme. The recombinant nucleic acid may be incorporated into the genome of the cell or may be contained on an extra-chromosomal plasmid. Techniques for genetic manipulation are described in further detail below. The genetically modified yeasts of the invention are also referred to herein as “recombinant,” “engineered,” or “bioengineered” yeasts, or other designations.

The recombinant yeasts of the invention may comprise one or more recombinant nucleic acids configured to express any one or more of the following enzymes in any combination: an acetyl-CoA thioesterase, an acetyl-CoA C-acetyltransferase, an acetoacetyl-CoA transferase, an acetoacetyl-CoA thioesterase, an acetoacetate decarboxylase, and an isopropanol dehydrogenase. The one or more recombinant nucleic acids preferably comprise one or more recombinant genes configured to express the above-referenced enzymes. In some versions, each enzyme is expressed from a separate recombinant gene. In some versions, one of more of the enzymes is expressed from a single gene in the form of an artificial operon. In some versions, one or more of the enzymes may be localized in the peroxisome.

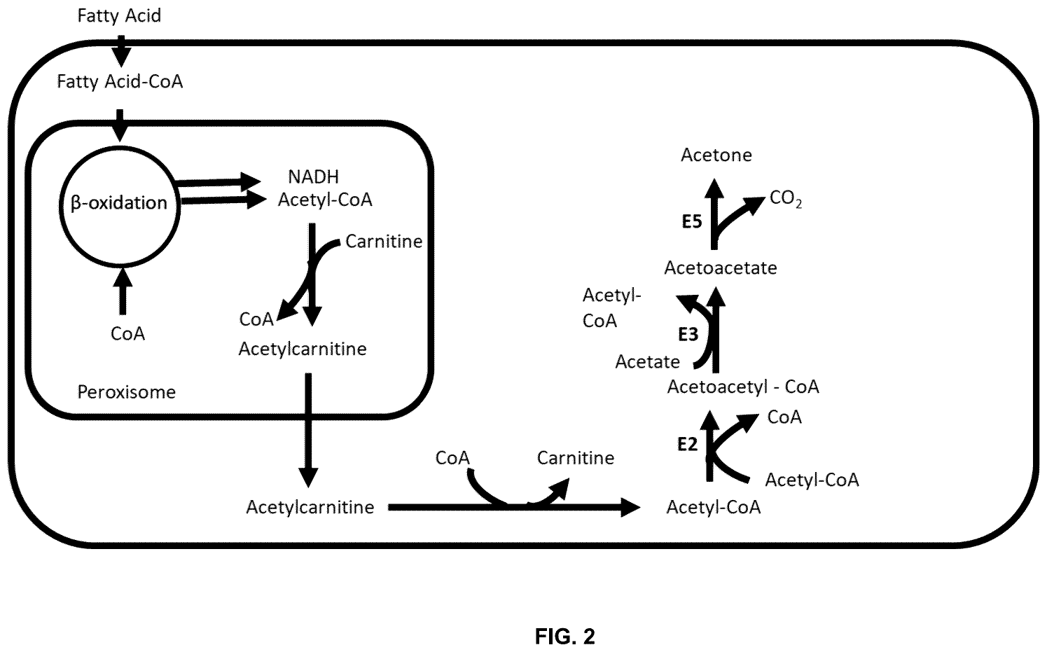

In one embodiment, as illustrated in FIG. 2, a recombinant yeast is constructed that expresses an acetoacetyl-CoA transferase (E3 activity) and an acetoacetate decarboxylase (E5 activity). Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

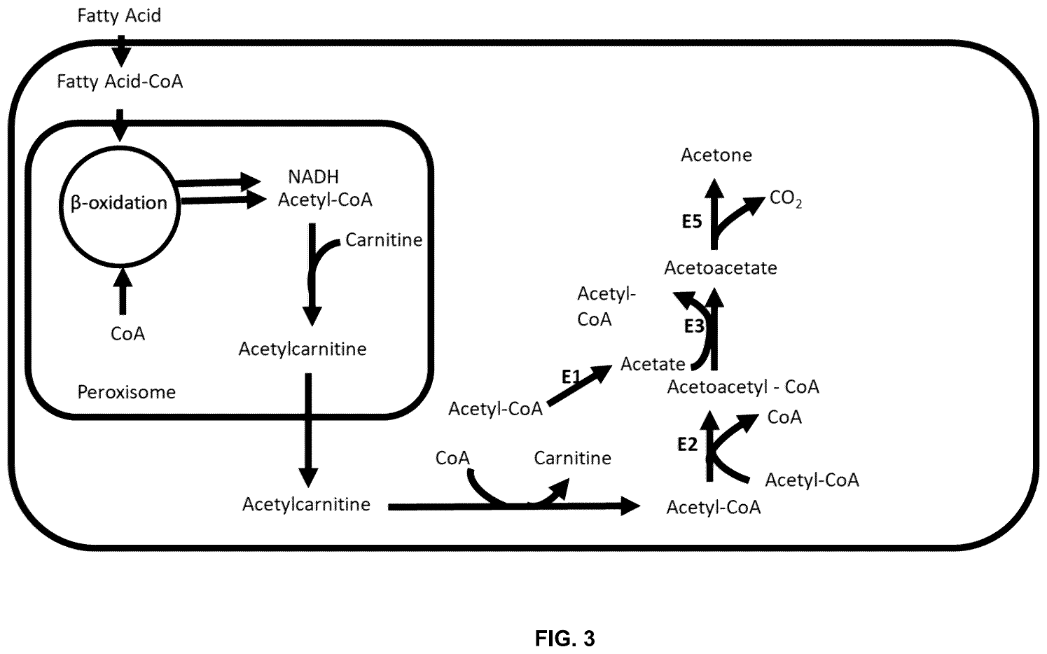

In another embodiment, as illustrated in FIG. 3, a recombinant yeast is constructed that expresses an acetyl-CoA thioesterase (E1 activity), an acetoacetyl-CoA transferase (E3 activity), and an acetoacetate decarboxylase (E5 activity). Genes encoding these enzymes can be placed under a strong or a fatty acid inducible promoter and integrated into the genome. Other genetic manipulations may be used to increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

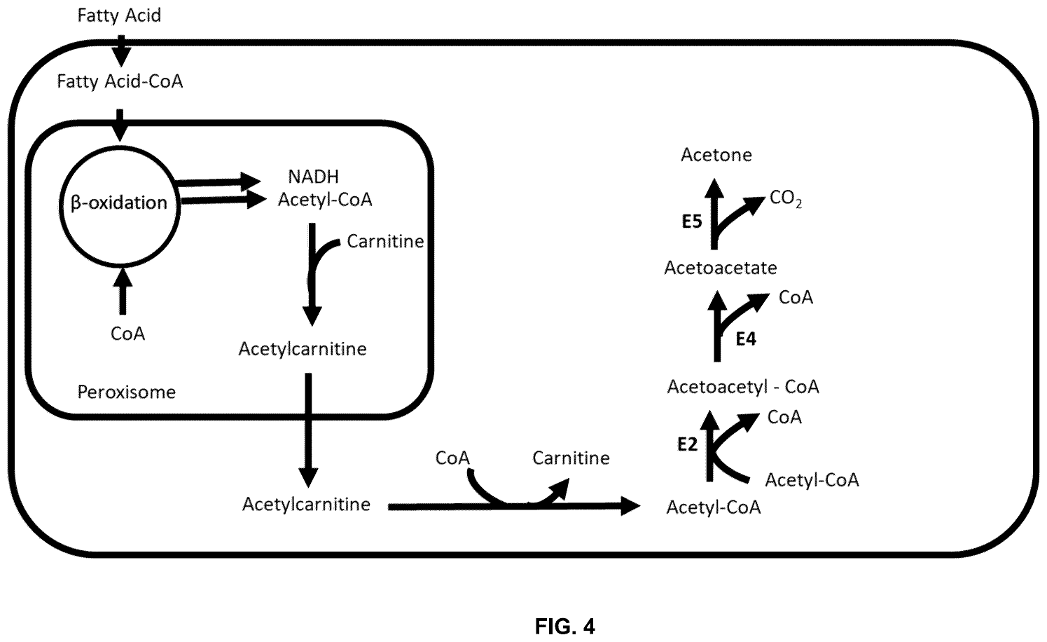

In another embodiment, as illustrated in FIG. 4, a recombinant yeast is constructed that expresses an acetoacetyl-CoA thioesterase (E4 activity) and an acetoacetate decarboxylase (E5 activity). Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

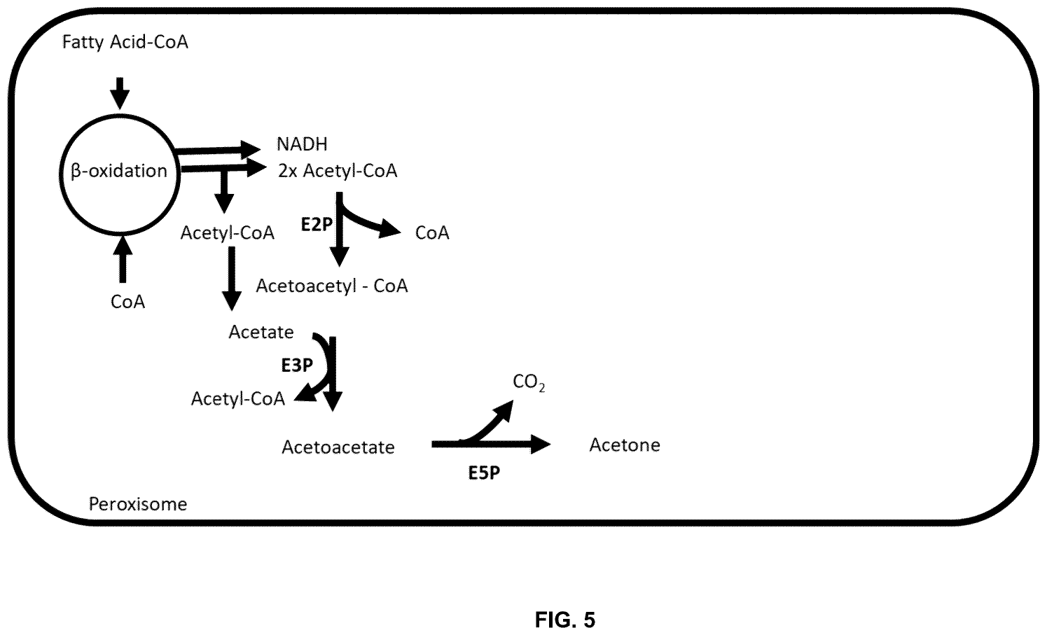

In another embodiment, as illustrated in FIG. 5, a recombinant yeast is constructed that expresses an acetoacetyl-CoA transferase (E3 activity) and an acetoacetate decarboxylase (E5 activity) in the peroxisome. These enzymes are targeted to the peroxisome by adding an N-terminal or C-terminal signal. An example of a C-terminal signal is three amino acids Serine Lysine and Leucine (SKL) (SEQ ID NO:186) or GRRAKL (SEQ ID NO:187). Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

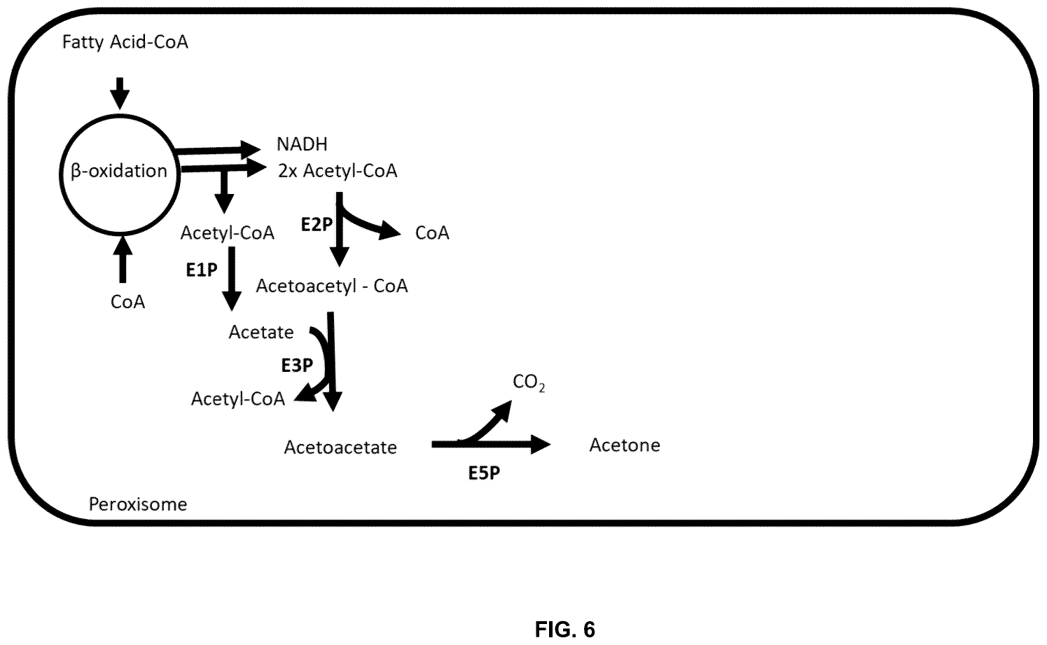

In another embodiment, as illustrated in FIG. 6, a recombinant yeast is constructed that expresses an acetyl-CoA thioesterase (E1 activity), an acetoacetyl-CoA transferase (E3 activity), and an acetoacetate decarboxylase (E5 activity) in the peroxisome. These enzymes are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

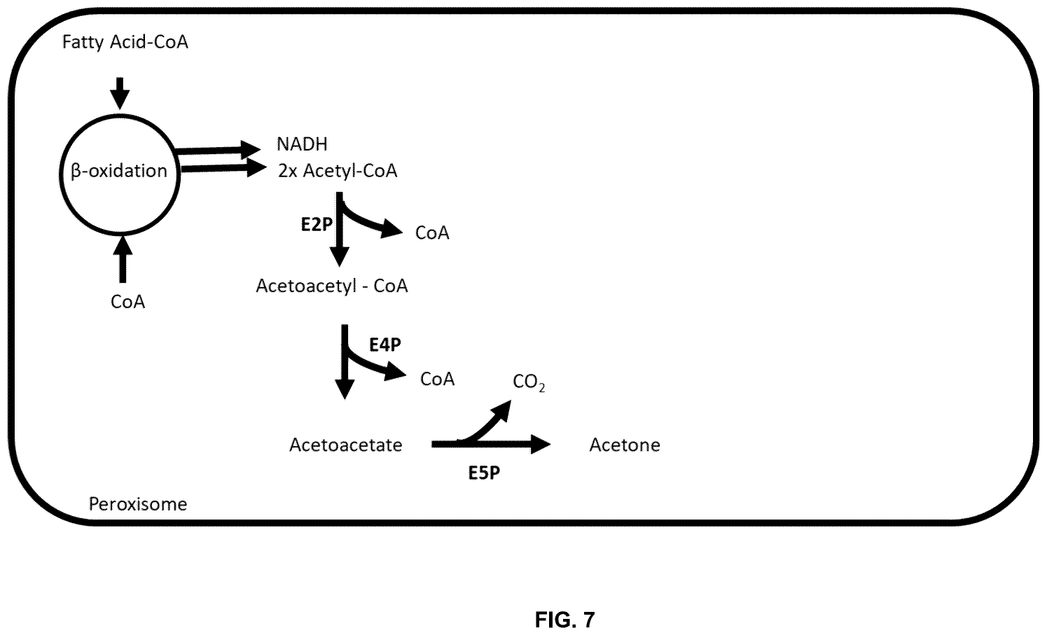

In another embodiment, as illustrated in FIG. 7, a recombinant yeast is constructed that expresses an acetoacetyl-CoA thioesterase (E4 activity) and an acetoacetate decarboxylase (E5 activity) in the peroxisome. These enzymes are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

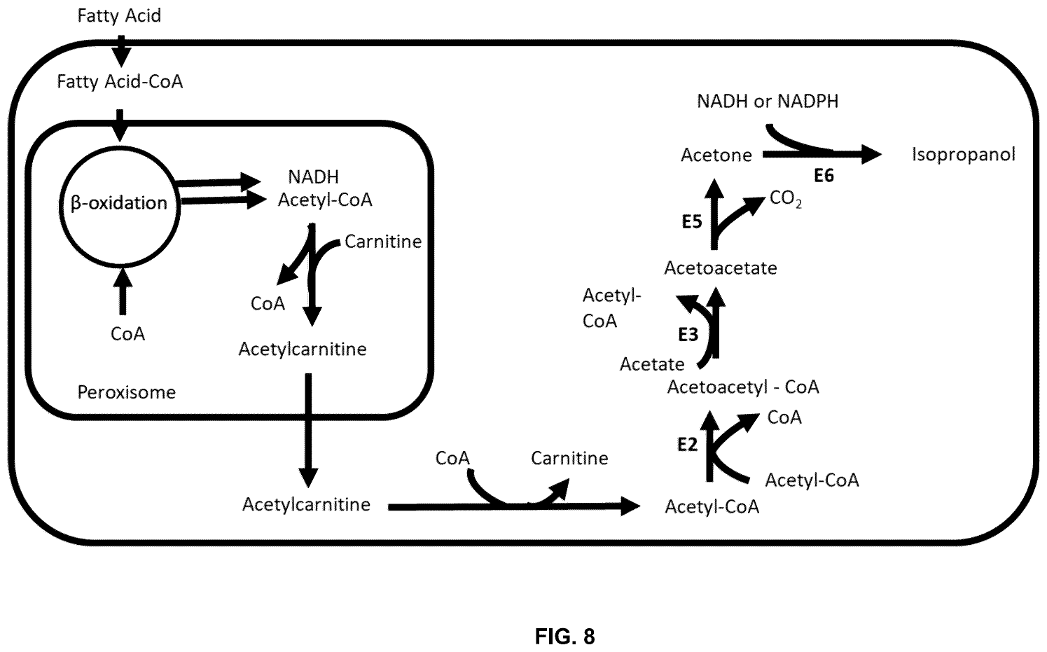

In another embodiment, as illustrated in FIG. 8, a recombinant yeast is constructed that expresses an acetoacetyl-CoA transferase (E3 activity), an acetoacetate decarboxylase (E5 activity), and an isopropanol dehydrogenase (E6 activity). Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

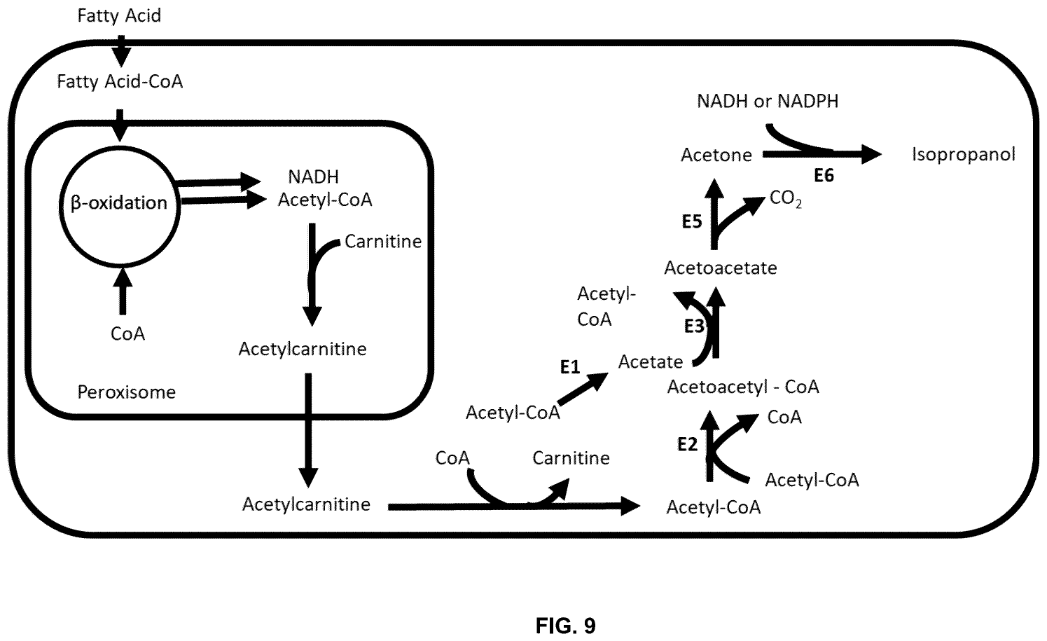

In another embodiment, as illustrated in FIG. 9, a recombinant yeast is constructed that expresses an acetyl-CoA thioesterase (E1 activity), an acetoacetyl-CoA transferase (E3 activity), an acetoacetate decarboxylase (E5 activity), and an isopropanol dehydrogenase (E6 activity). Genes encoding these enzymes can be placed under a strong or a fatty acid inducible promoter and integrated into the genome. Other genetic manipulations may increase flux towards the acetoacetylCOA intermediate. For example, an acetyl-CoA C-acetyltransferase (E2 activity) may be overexpressed. An endogenous enzyme or a similar enzyme from a different organism may be used as the enzyme source.

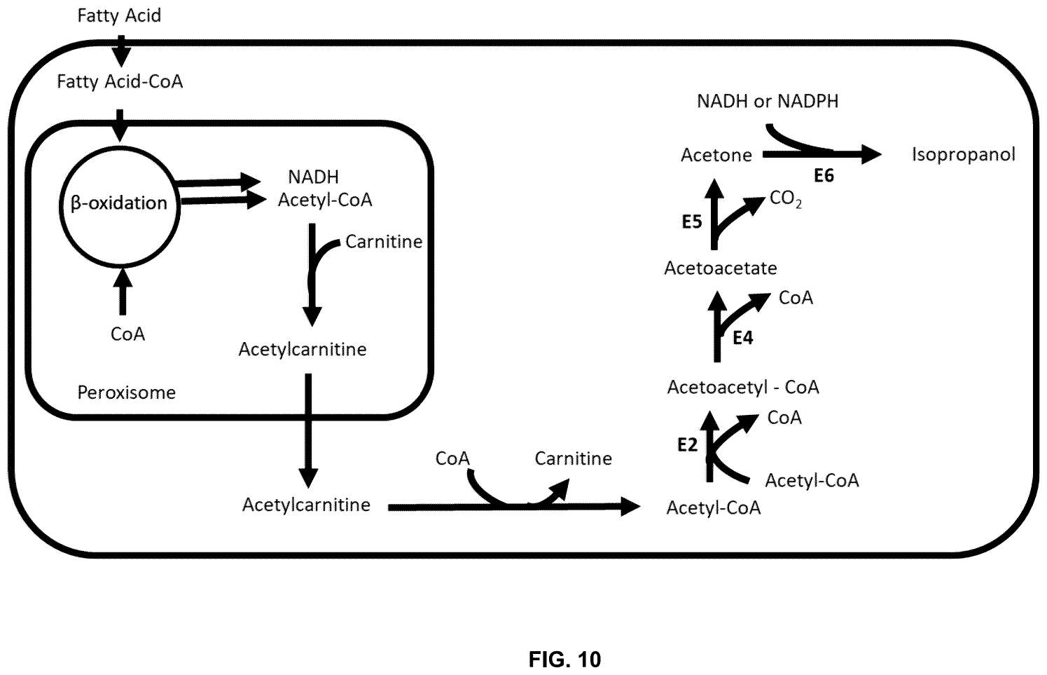

In another embodiment, as illustrated in FIG. 10, a recombinant yeast is constructed that expresses an acetoacetyl-CoA thioesterase (E4 activity), an acetoacetate decarboxylase (E5 activity), and an isopropanol dehydrogenase (E6 activity). Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

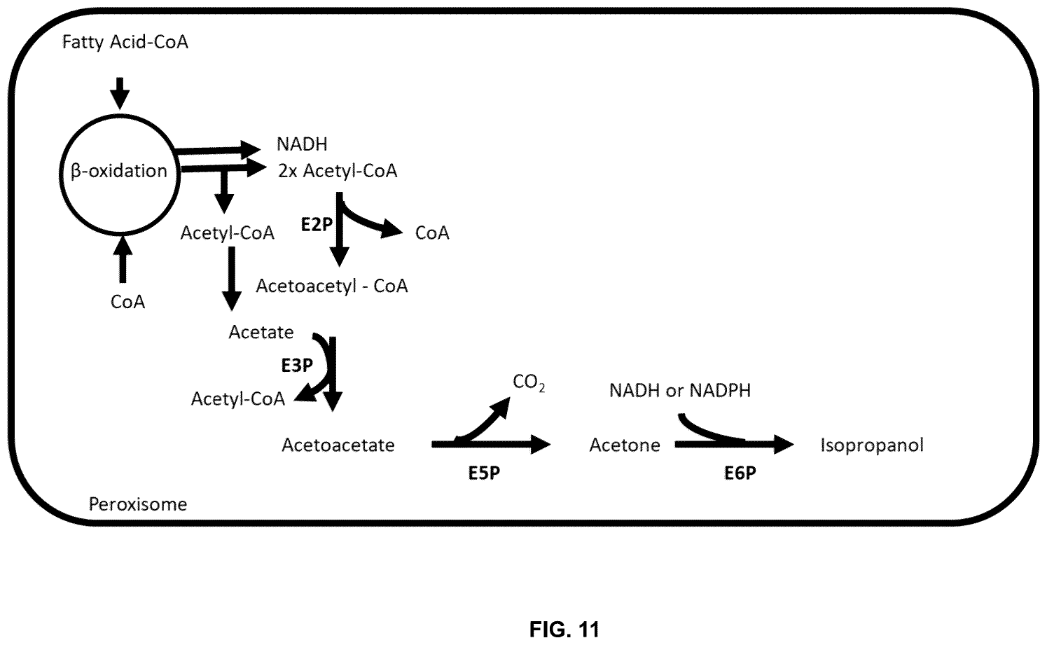

In another embodiment, as illustrated in FIG. 11, a recombinant yeast is constructed that expresses an acetoacetyl-CoA transferase (E3 activity), an acetoacetate decarboxylase (E5 activity), and an isopropanol dehydrogenase (E6 activity) in the peroxisome. These enzymes are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

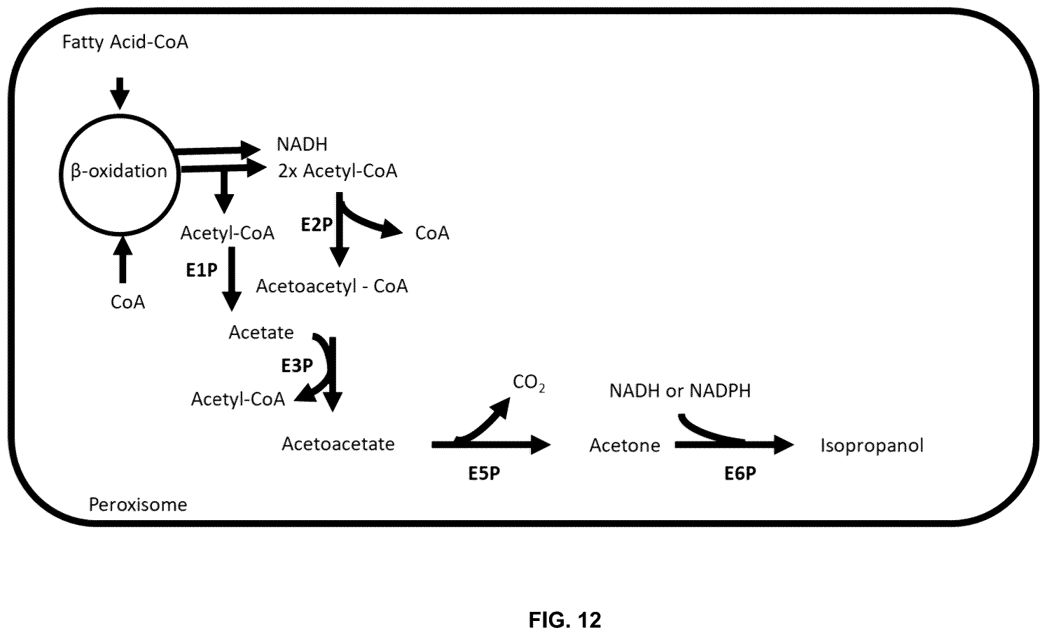

In another embodiment, as illustrated in FIG. 12, a recombinant yeast is constructed that expresses an acetyl-CoA thioesterase (E1 activity), an acetoacetyl-CoA transferase (E3 activity), an acetoacetate decarboxylase (E5 activity), and an isopropanol dehydrogenase (E6 activity) in the peroxisome. These enzymes are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

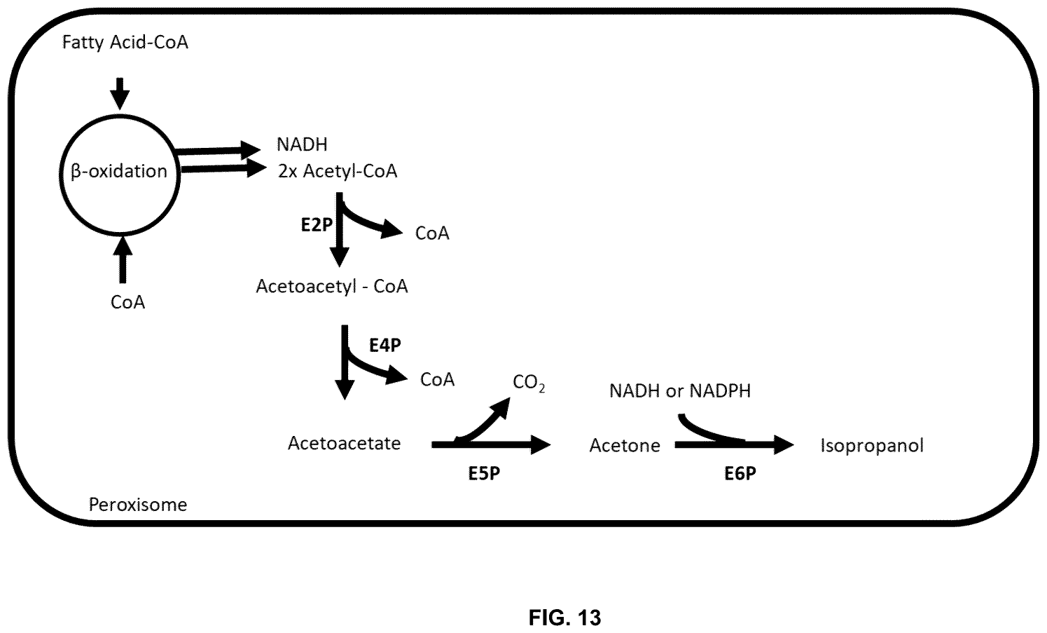

In another embodiment, as illustrated in FIG. 13, a recombinant yeast is constructed that expresses an acetoacetyl-CoA thioesterase (E4 activity), an acetoacetate decarboxylase (E5 activity), and an isopropanol dehydrogenase (E6 activity) in the peroxisome. These enzymes are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source. The protein is targeted to the peroxisome by adding an N-terminal or C-terminal signal.

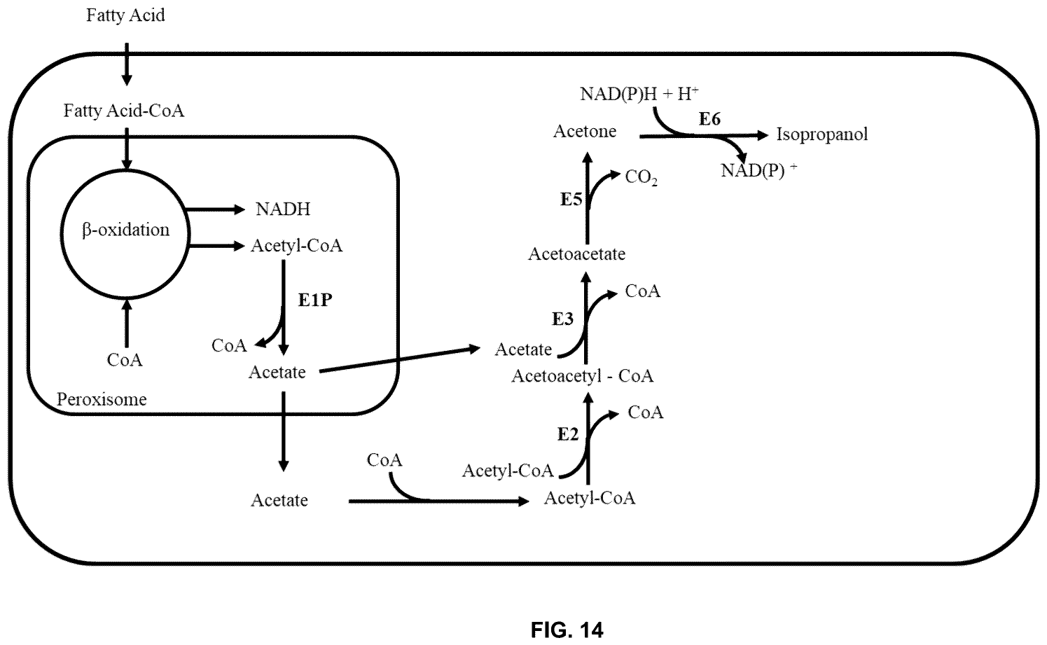

In another embodiment, as illustrated in FIG. 14, a recombinant yeast is constructed that expresses an acetyl-CoA thioesterase (E1 activity) in the peroxisome and expressing an acetoacetyl-CoA transferase (E3 activity), an acetoacetate decarboxylase (E5 activity), and an isopropanol dehydrogenase (E6 activity) in the cytoplasm. The acetyl-CoA thioesterase (E1 activity) is targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

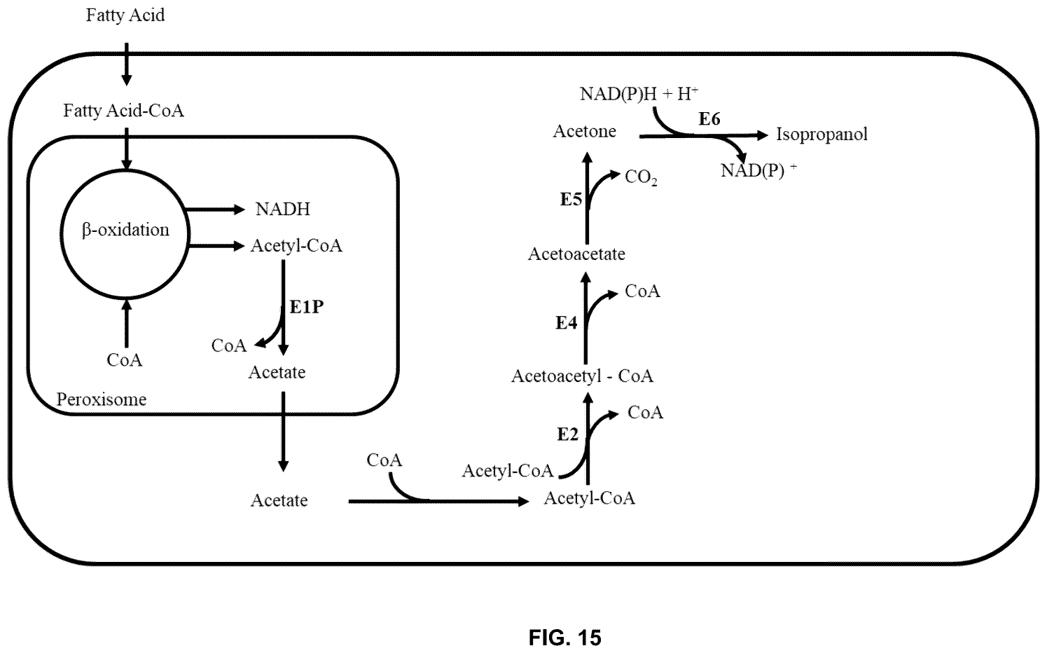

In another embodiment, as illustrated in FIG. 15, a recombinant yeast is constructed that expresses an acetyl-CoA thioesterase (E1 activity) in the peroxisome and expressing an acetoacetyl-CoA thioesterase (E4 activity), an acetoacetate decarboxylase (E5 activity), and an isopropanol dehydrogenase (E6 activity) in the cytoplasm. The acetyl-CoA thioesterase (E1 activity) is targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed. An endogenous enzyme or a similar enzyme from a different organism may be used as the enzyme source.

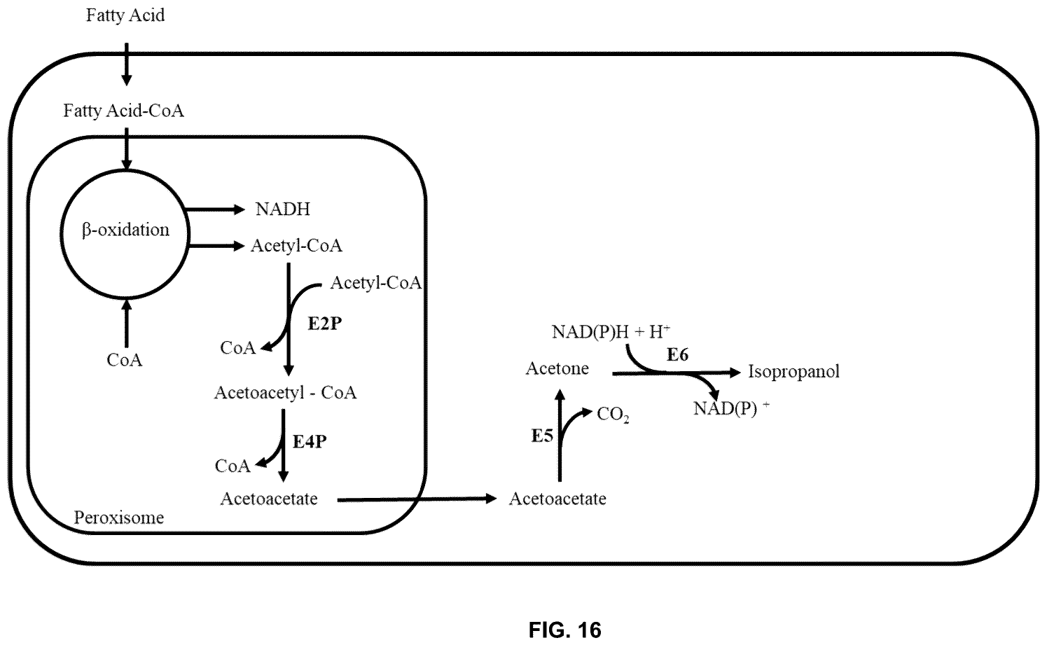

In another embodiment, as illustrated in FIG. 16, a recombinant yeast is constructed that expresses an acetoacetyl-CoA thioesterase (E4 activity) in the peroxisome and expressing an acetoacetate decarboxylase (E5 activity) and an isopropanol dehydrogenase (E6 activity) in the cytoplasm. The acetoacetyl-CoA thioesterase (E4 activity) is targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed in the peroxisome. An endogenous enzyme or a similar enzyme from a different organism may be used as the enzyme source.

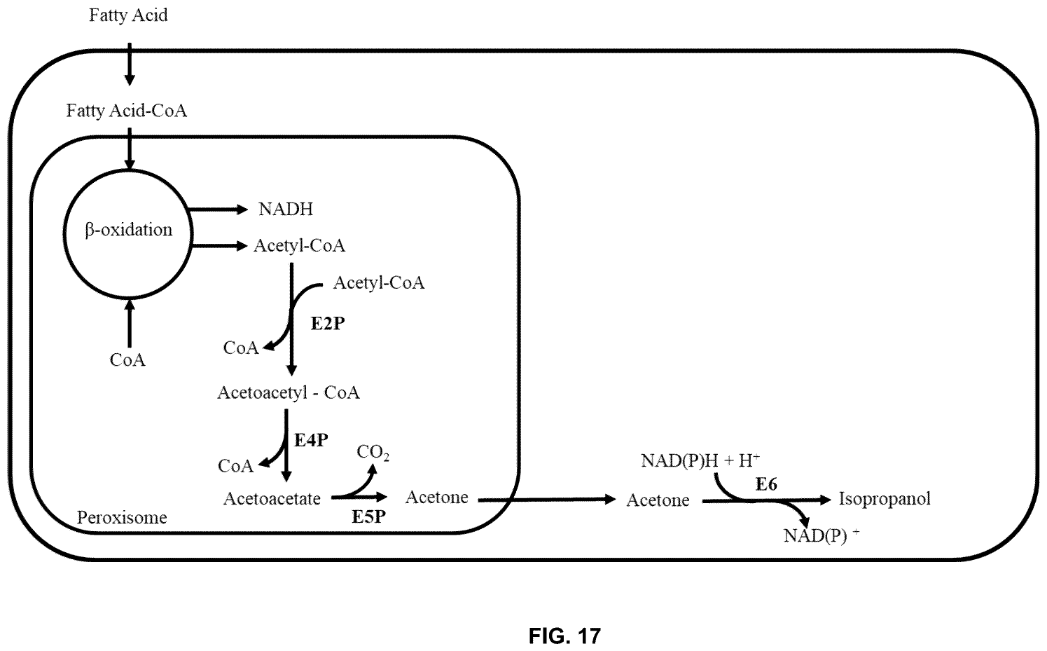

In another embodiment, as illustrated in FIG. 17, a recombinant yeast is constructed that expresses an acetoacetyl-CoA thioesterase (E4 activity) and an acetoacetate decarboxylase (E5 activity) in the peroxisome and expressing and an isopropanol dehydrogenase (E6 activity) in the cytoplasm. The acetoacetyl-CoA thioesterase (E4 activity) and acetoacetate decarboxylase (E5 activity) are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed in the peroxisome. An endogenous enzyme or a similar enzyme from a different organism may be used as the enzyme source.

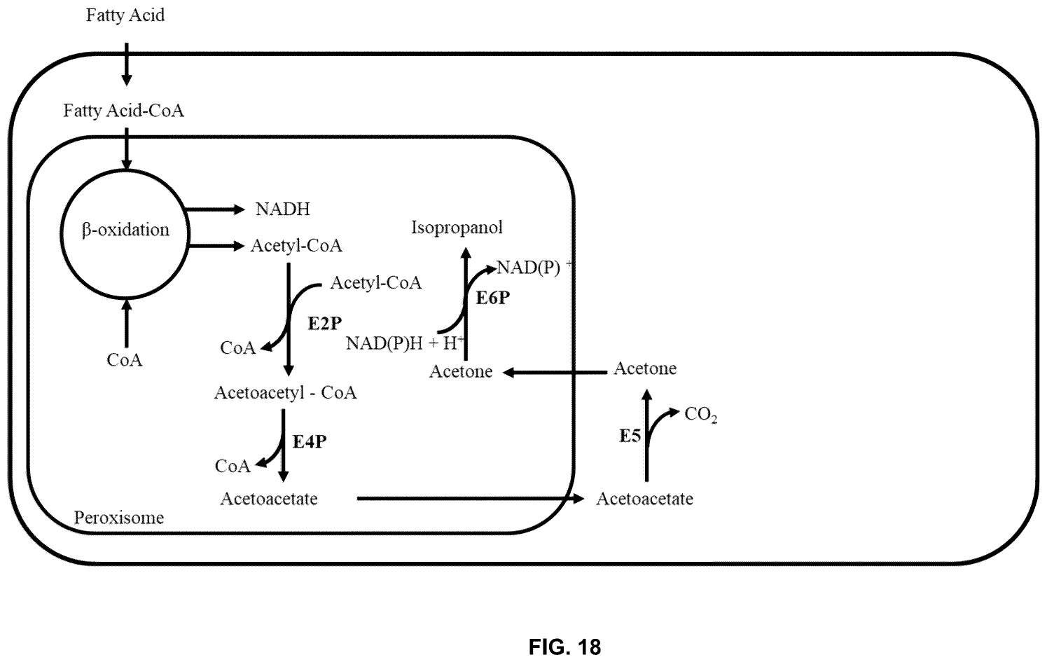

In another embodiment, as illustrated in FIG. 18, a recombinant yeast is constructed that expresses an acetoacetyl-CoA thioesterase (E4 activity) and an isopropanol dehydrogenase (E6 activity) in the peroxisome and expressing an acetoacetate decarboxylase (E5 activity) in the cytoplasm. The acetoacetyl-CoA thioesterase (E4 activity) and isopropanol dehydrogenase (E6 activity) are targeted to the peroxisome by adding an N-terminal or C-terminal signal. Genes encoding these enzymes can be placed under a strong or a fatty acid inducible promoter and integrated into the genome. Other genetic manipulations may increase flux towards the acetoacetyl-CoA intermediate. For example, an acetyl-CoA C-acetyltransferase (E2 activity) may be overexpressed in the peroxisome. An endogenous enzyme or a similar enzyme from a different organism may be used as the enzyme source.

Acetyl-CoA thioesterases (E1 activity) include enzymes falling under Enzyme Commission (EC) number 3.1.2.1. Exemplary acetyl-CoA thioesterases that may be expressed include ThACTI (SEQ ID NOs:1-3) encoded by TbACT1 (SEQ ID NOs:4-6) from Trypanosoma brucei. The recombinant yeasts of the invention in some versions can comprise one or more recombinant genes configured to express an acetyl-CoA thioesterase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs: 1-3.

Acetyl-CoA C-acetyltransferases (E2 activity) include enzymes falling under EC number 2.3.1.9. Exemplary acetyl-CoA C-acetyltransferases that may be expressed include CVERG10 (SEQ ID NOs:7-8) encoded by (VERG10 (SEQ ID NOs:11-12) from Candida viswanathii, YIERG10 (SEQ ID NO:9) encoded by YIERG10 (SEQ ID NO:13) from Yarrowia lipolytica CLIB122, and CjERG10 (SEQ ID NO:10) encoded by CjERG10 (SEQ ID NO:14) from Cyberlindnera jadinii. The recombinant yeasts of the invention in some versions can comprise one or more recombinant genes configured to express an acetyl-CoA C-acetyltransferase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs: 7-10.

Acetoacetyl-CoA transferases (E3 activity) include enzymes falling under EC number 2.8.3.9. Exemplary acetoacetyl-CoA transferases that may be expressed include CbCTF1 (SEQ ID NOs:15-16) encoded by CbCTF1 (SEQ ID NOs:27-28) and (bCTF2 (SEQ ID NOs:17-18) encoded by (bCTF2 (SEQ ID NOs:29-30) from a species of Clostridium, CsCTF1 (SEQ ID NO: 19) encoded by (SCTFI (SEQ ID NO:31) and CsCTF2 (SEQ ID NO:20) encoded by CsCTF2 (SEQ ID NO:32) from Clostridium saccharobutylicum, CaCTF1 (SEQ ID NO:21) encoded by CaCTF1 (SEQ ID NO:33) and CaCTF2 (SEQ ID NO:22) encoded by Ca(TF2 (SEQ ID NO:34) from Clostridium algidicarnis, (CTF1 (SEQ ID NO:23) encoded by (CTF1 (SEQ ID NO:35) and (CTF2 (SEQ ID NO:24) encoded by (CTF2 (SEQ ID NO:36) from Clostridium thermoalcaliphilum, and EcCTF1 (SEQ ID NO:25) encoded by EcCTF1 (SEQ ID NO:37) and EcCTF2 (SEQ ID NO:26) encoded by Ec(TF2 (SEQ ID NO:38) from Escherichia coli. The recombinant yeasts of the invention in some versions can comprise one or more recombinant genes configured to express an acetoacetyl-CoA transferase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs: 15-26.

Acetoacetyl-CoA thioesterases (E4 activity) include enzymes falling under EC number 3.1.2. Exemplary acetoacetyl-CoA thioesterases that may be expressed include TbACT1 (SEQ ID NOs:1-3) encoded by TbACT1 (SEQ ID NOs:4-6) from Trypanosoma brucei, HiybgC (SEQ ID NO:39) encoded by HiybgC (SEQ ID NO:51) from Haemophilus influenzae, HhybgC (SEQ ID NO:40) encoded by HhybgC (SEQ ID NO:52) from Haemophilus haemolyticus, HpybgC (SEQ ID NO:41) encoded by HpybgC (SEQ ID NO:53) from Haemophilus parainfluenzae, RtybgC (SEQ ID NO:42) encoded by RtybgC (SEQ ID NO:54) from Rodentibacter trehalosifermentans, RmybgC (SEQ ID NO:43) encoded by RmybgC (SEQ ID NO:55) from Rodentibacter myodis, RgYBGC (SEQ ID NO:44) encoded by RgYBGC′ (SEQ ID NO:56) from Rodentibacter genomosp. 2, BxSrfAD (SEQ ID NO:45) encoded by BxSrfAD) (SEQ ID NO:57) from a species of Bacillus, BaSrfAD (SEQ ID NO:46) encoded by BaSrfAD (SEQ ID NO:58) from Bacillus atrophaeus, BhSrfAD (SEQ ID NO:47) encoded by BhSrfAD) (SEQ ID NO:59) from Bacillus halotolerans, SmACL (SEQ ID NO:48) encoded by SmACL (SEQ ID NO:60) from Sinorhizobium meliloti, ExACL (SEQ ID NO:49) encoded by ExACI. (SEQ ID NO:61) from a species of Ensifer, SfACL (SEQ ID NO:50) encoded by SfACI. (SEQ ID NO:62) from Sinorhizobium fredii. The recombinant yeasts of the invention in some versions can comprise one or more recombinant genes configured to express an acetoacetyl-CoA thioesterase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs: 1-3 and 39-50.

Acetoacetate decarboxylases (E5 activity) include enzymes falling under EC number 4.1.1.4. Exemplary acetoacetate decarboxylases that may be expressed include (bADC1 (SEQ ID NOs:63-66) encoded by (bADC1 (SEQ ID NOs:83-86) from Clostridium beijerinckii, CxADC1 (SEQ ID NO:67) encoded by (xADC1 (SEQ ID NO:87) from Clostridium sp. BL-8, CgADC1 (SEQ ID NO:68) encoded by (‘xADC’I (SEQ ID NO:88) from Clostridium gasigenes, CaADC1 (SEQ ID NO:69) encoded by CaADC1 (SEQ ID NO:89) from Clostridium acidisoli, PxADC1 (SEQ ID NO:70) encoded by PxADC1 (SEQ ID NO:90) from Paenibacillus sp. ovo31, HsADC1 (SEQ ID NO:71) encoded by HsAD(1 (SEQ ID NO:91) from Heyndrickx sporothermodurans, WxADC1 (SEQ ID NO:72) encoded by WxADC1 (SEQ ID NO:92) from Variovorax sp. KK3, (cADC1 (SEQ ID NO:73) encoded by (cADC1 (SEQ ID NO:93) from Clostridium cagae, CpADC1 (SEQ ID NO:74) encoded by (pADC1 (SEQ ID NO:94) from Clostridium pasteurianum, PmADC1 (SEQ ID NO:75) encoded by PmADC1 (SEQ ID NO:95) from Priestia megaterium, BaADC1 (SEQ ID NO:76) encoded by BaADC1 (SEQ ID NO:96) from Bacillus acidicola, PfADC1 (SEQ ID NO:77) encoded by PfADC1 (SEQ ID NO:97) from Pelosinus fermentans, CeADC1 (SEQ ID NOs: 78-79) encoded by CeADC1 (SEQ ID NOs:98-99) from Clostridium estertheticum, BxADC1 (SEQ ID NO:80) encoded by BxADC1 (SEQ ID NO:100) from Brevibacterium, CdADC1 (SEQ ID NO:81) encoded by CdADC1 (SEQ ID NO: 101) from Clostridium sp DL-VIII, CsADC1 (SEQ ID NO:82) encoded by CsADC1 (SEQ ID NO:102) from Clostridium saccharoperbutylacetonicum. The recombinant yeasts of the invention in some versions can comprise one or more recombinant genes configured to express an acetoacetate decarboxylase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs:63-82.

Isopropanol dehydrogenases (E6 activity) include enzymes falling under EC number 1.1.1.80. Exemplary isopropanol dehydrogenases that may be expressed include CbADH1 (SEQ ID NOs:103-104) encoded by (bADHI (SEQ ID NOs:110-111) from Clostridium beijerinckii, NeADHI (SEQ ID NOs:105-106) encoded by NeADHI (SEQ ID NOs:112-113) from Neurospora crassa, CpADH1 (SEQ ID NOs:107-108) encoded by CpADH1 (SEQ ID NOs: 114-115) from Candida parapsilosis, and CvADH5 (SEQ ID NO:109) encoded by (vADHI (SEQ ID NO:116) from Candida viswanathii. The recombinant yeasts of the invention in some versions can comprise one or more recombinant genes configured to express isopropanol dehydrogenase comprising a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of SEQ ID NOs: 103-109.

Suitable enzymes that may be expressed from the recombinant genes of the invention include those comprising polypeptide sequences at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of sequences listed above or elsewhere herein. Other suitable enzymes that may be expressed include orthologs and paralogs of the enzymes listed above. Other suitable enzymes that may be expressed include those comprising polypeptide sequences at least about 80%, at least about 85%, at least about 90%, at least about 95%, at least about 97%, or at least about 99% identical to orthologs and paralogs of the enzymes listed above. The orthologs are preferably from yeasts, such as any of the yeasts described herein. The recombinant gene encoding the enzymes may include introns or be devoid of introns or any or all other non-coding regions in the native gene. Any nucleotide sequences capable of expressing the polypeptide sequences encompassed herein are acceptable. Tremendous variation from the exemplary nucleotide sequences described herein is possible due to the redundancy in the genetic code and codon optimization.

Coding sequences of the above-mentioned enzymes in the recombinant genes are preferably operably linked to a promoter. The promoter may be a heterologous promoter. The promoter may be a fatty acid inducible promoter or strong promoter. “Fatty acid promoter” refers to an inducible promoter that is induced by fatty acids. The promoter may be a constitutive promoter or an inducible promoter. The promoter can be heterologous to the coding sequence. Exemplary promoters that may be operably linked to the coding sequences of the above-mentioned enzymes include the POX4 (Acyl-CoA Oxidase 4) promoter (SEQ ID NO:117), the PEX11 (Peroxisome Membrane Protein) promoter (SEQ ID NO:118), the ETF1 (Elongation Transcription Factor 1 alpha) promoter (SEQ ID NO:119), the TDH3 (Glyceraldehyde-3-Phosphate Dehydrogenase, isozyme 3) promoter (SEQ ID NO:120), the ACTI (Actin) promoter (Da Silva & Srikrishnan, FEMS Yeast Res. 2012, 12: 197-214), the POX5 (Acyl-CoA Oxidase 5) promoter (Juretzek et al., Biotechnol. Bioprocess Eng. 2000, 5: 320-326) or sequence variants at least about at least about 80%, at least about 85%, at least about 90%, at least about 95%, at least about 97%, or at least about 99% identical thereto. A non-limiting list of fatty acid inducible promoters that may be operably lined to the coding sequences of the above-mentioned enzymes includes the POX1 (Acyl-CoA Oxidase 1) promoter, the POX2 (Acyl-CoA Oxidase 2) promoter, the POX4 (Acyl-CoA Oxidase 4) promoter, the POX5 (Acyl-CoA Oxidase 5) promoter, the G3P (Glycerol-3-Phosphate Dehydrogenase) promoter, the ICL1 (Isocitrate Lyase) promoter, the POT1 (3-Oxo-Acyl-CoA Thiolase) promoter, the LIP2 (Lipoyl ligase) promoter, the TDH1 (Glyceraldehyde-3-Phosphate Dehydrogenase, isozyme 1) promoter, the TDH3 (Glyceraldehyde-3-Phosphate Dehydrogenase, isozyme 3) promoter, and the PEX11 (Peroxisome Membrane Protein) promoter (Juretzek et al., Biotechnol. Bioprocess Eng. 2000, 5: 320-326; Trassaer et al. Microb Cell Fact 2017, 16: 141; Han et al., Eng Life Sci. 2020, 20: 186-196; Yazaw et al., Appl Environ Microbiol 2007, 73(21): 6965-6971; Dyer et al., Appl Microbiol Biotechnol 2002, 59: 224-230), and variants of any of the foregoing. The variants preferably comprise a sequence at least about 80%, at least about 85%, at least about 90%, at least about 95%, at least about 97%, or at least about 99% identical to any of the foregoing.

Coding sequences of the above-mentioned enzymes in the recombinant genes are preferably operably linked to a terminator. The terminator can be heterologous to the coding sequence. Exemplary terminators that may be operably linked to the coding sequences of the above-mentioned enzymes include the POX4 terminator (SEQ ID NO:121), the ETF1 terminator (SEQ ID NO:122), or sequence variants at least about at least about 80%, at least about 85%, at least about 90%, at least about 95%, at least about 97%, or at least about 99% identical thereto.

The recombinant yeasts of the invention with the modifications described herein preferably exhibit a property selected from the group consisting of increased acetone production, increased acetone secretion, increased isopropanol production, and increased isopropanol secretion relative to a non-recombinant control.

The recombinant yeasts of the invention may be genetically altered to express or overexpress any of the specific genes or gene products explicitly described herein or homologs thereof. Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Nucleic acid or gene product (amino acid) sequences of any known gene, including the genes or gene products described herein, can be determined by searching any sequence databases known in the art using the gene name or accession number as a search term. Common sequence databases include GenBank (www.ncbi.nlm.nih.gov), ExPASy (expasy.org), KEGG (www.genome.jp), among others. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 35% 40%, 45% 50%, 55%, 60%, 65%, 70%, 75%, 80%, 85%, 90%, 95%, or 99% or more, can also be used to establish homology. Accordingly, homologs of the genes or gene products described herein include genes or gene products having at least about 30%, 35%, 40%, 45%, 50%, 55%, 60%, 65%, 70%, 75%, 80%, 85%, 90%, 95%, or 99% identity to the genes or gene products described herein. Methods for determining sequence similarity percentages (e.g., BLASTP and BLASTN using default parameters) are described herein and are generally available. The homologous proteins should demonstrate comparable activities and, if an enzyme, participate in the same or analogous pathways. Homologs include orthologs and paralogs. “Orthologs” are genes and products thereof in different species that evolved from a common ancestral gene by speciation. Normally, orthologs retain the same or similar function in the course of evolution. Paralogs are genes and products thereof related by duplication within a genome. As used herein, “orthologs” and “paralogs” are included in the term “homologs.”

For sequence comparison and homology determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is a nucleic acid or amino acid sequence corresponding to the genes or gene products described herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described above (or other algorithms available to persons of skill) or by visual inspection.

The phrase “substantially identical” in the context of two nucleic acids or polypeptides refers to two or more sequences or subsequences that have at least about 60%, about 65%, about 70%, about 75%, about 80%, about 85%, about 90, about 95%, about 98%, or about 99% or more nucleotide or amino acid residue identity, when compared and aligned for maximum correspondence, as measured using a sequence comparison algorithm or by visual inspection. Such “substantially identical” sequences are typically considered to be “homologous”, without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, at least about 250 residues, or over the full length of the two sequences to be compared.

Terms used herein pertaining to genetic manipulation are defined as follows.

Deletion: The removal of one or more nucleotides from a nucleic acid molecule or one or more amino acids from a protein, the regions on either side being joined together.

Derived: When used with reference to a nucleic acid or protein, “derived” means that the nucleic acid or polypeptide is isolated from a described source or is at least 70%, 80%, 90%, 95%, 99%, or more identical to a nucleic acid or polypeptide included in the described source.

Endogenous: As used herein with reference to a nucleic acid molecule, genetic element (e.g., gene, promoter, etc.), or polypeptide in a particular cell, “endogenous” refers to a nucleic acid molecule, genetic element, or polypeptide that is in the cell and was not introduced into the cell or transferred within the genome of the cell using recombinant engineering techniques. For example, an endogenous genetic element is a genetic element that was present in a cell in its particular locus in the genome when the cell was originally isolated from nature.

Exogenous: As used herein with reference to a nucleic acid molecule, genetic element (e.g., gene, promoter, etc.), or polypeptide in a particular cell, “exogenous” refers to any nucleic acid molecule, genetic element, or polypeptide that was introduced into the cell or transferred within the genome of the cell using recombinant engineering techniques. For example, an exogenous genetic element is a genetic element that was not present in its particular locus in the genome when the cell was originally isolated from nature.

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Introduce: When used with reference to genetic material, such as a nucleic acid, and a cell, “introduce” refers to the delivery of the genetic material to the cell in a manner such that the genetic material is capable of being expressed within the cell. Introduction of genetic material includes both transformation and transfection. Transformation encompasses techniques by which a nucleic acid molecule can be introduced into cells such as prokaryotic cells or non-animal eukaryotic cells. Transfection encompasses techniques by which a nucleic acid molecule can be introduced into cells such as animal cells. These techniques include but are not limited to introduction of a nucleic acid via conjugation, electroporation, lipofection, infection, and particle gun acceleration.

Isolated: An “isolated” biological component (such as a nucleic acid molecule, polypeptide, or cell) has been substantially separated or purified away from other biological components in which the component naturally occurs, such as other chromosomal and extrachromosomal DNA and RNA and proteins. Nucleic acid molecules and polypeptides that have been “isolated” include nucleic acid molecules and polypeptides purified by standard purification methods. The term also includes nucleic acid molecules and polypeptides prepared by recombinant expression in a cell as well as chemically synthesized nucleic acid molecules and polypeptides. In one example, “isolated” refers to a naturally-occurring nucleic acid molecule that is not immediately contiguous with both of the sequences with which it is immediately contiguous (one on the 5′ end and one on the 3′ end) in the naturally-occurring genome of the organism from which it is derived.

Gene: Genes minmally include a promoter operationally linked to a coding sequence, and can include other elements that facilitate or regulate the transcription and/or translation of the coding sequence.

Heterologous: The term “heterologous” refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature. A secretion signal sequence that is heterologous to a given protein (such as an enzyme) is a secretion signal sequence that is not operably linked with the protein in nature.

Nucleic acid: Encompasses both RNA and DNA molecules including, without limitation, cDNA, genomic DNA, and mRNA. Nucleic acids also include synthetic nucleic acid molecules, such as those that are chemically synthesized or recombinantly produced. The nucleic acid can be double-stranded or single-stranded. Where single-stranded, the nucleic acid molecule can be the sense strand, the antisense strand, or both. In addition, the nucleic acid can be circular or linear.

Operably linked: A first element is operably linked with a second element when the first element is placed in a functional relationship with the second element. For instance, a promoter is operably linked to a coding sequence if the promoter affects the transcription or expression of the coding sequence. A peroxisome targeting sequence is operably linked to a protein (such as an enzyme) when the peroxisome targeting sequence targets the enzyme to the peroxisome.

Overexpress: When a gene is caused to be transcribed at an elevated rate compared to the endogenous or basal transcription rate for that gene. In some examples, overexpression additionally includes an elevated rate of translation of the gene compared to the endogenous translation rate for that gene. Methods of testing for overexpression are well known in the art, for example transcribed RNA levels can be assessed using RT-PCR and protein levels can be assessed using SDS-PAGE gel analysis.

Recombinant: A recombinant nucleic acid or polypeptide is one comprising a sequence that is not naturally occurring. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in a corresponding native cell. A recombinant cell (such as a recombinant yeast) is one that comprises a recombinant nucleic acid, a recombinant gene, or a recombinant polypeptide.

Vector or expression vector: An entity comprising a nucleic acid molecule that is capable of introducing the nucleic acid, or being introduced with the nucleic acid, into a cell for expression of the nucleic acid. A vector can include nucleic acid sequences that permit it to replicate in the cell, such as an origin of replication. A vector can also include one or more selectable marker genes and other genetic elements known in the art. Examples of suitable vectors are found below.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below.

Exogenous nucleic acids can be introduced stably or transiently into a cell using techniques well known in the art, including electroporation, lithium acetate transformation, calcium phosphate precipitation, DEAE-dextran mediated transfection, liposome-mediated transfection, conjugation, transduction, and the like. For stable transformation, a nucleic acid can further include a selectable marker. Suitable selectable markers include antibiotic resistance genes that confer, for example, resistance to phleomycin, nourseothricin, G418, hygromycin B, neomycin, tetracycline, chloramphenicol, or kanamycin, genes that complement auxotrophic deficiencies, and the like. (See below for more detail.)

Various embodiments of the invention use an expression vector that includes a recombinant nucleic acid encoding a protein involved in a metabolic or biosynthetic pathway. Suitable expression vectors include, but are not limited to viral vectors, phage vectors, bacteriophage vectors, plasmids, phagemids, cosmids, fosmids, bacterial artificial chromosomes, Pl-based artificial chromosomes, yeast plasmids, yeast artificial chromosomes, and any other vectors specific for cells of interest.

Useful vectors can include one or more selectable marker genes to provide a phenotypic trait for selection of transformed cells. The selectable marker gene encodes a protein necessary for the survival or growth of transformed cells grown in a selective culture medium. Cells not transformed with the vector containing the selectable marker gene will not survive in the culture medium. Typical selection genes encode proteins that (a) confer resistance to antibiotics or other toxins, e.g., nourseothricin, G418, hygromycin B, ampicillin, neomycin, methotrexate, or tetracycline, (b) complement auxotrophic deficiencies, or (c) supply critical nutrients not available from complex media. In alternative embodiments, the selectable marker gene is one that encodes orotidine 5′-phosphate decarboxylase, dihydrofolate reductase or confers neomycin resistance (for use in eukaryotic cell culture).

The coding sequence in the expression vector is operably linked to an appropriate expression control sequence (promoters, enhancers, and the like) to direct synthesis of the encoded gene product. Such promoters can be derived from endogenous or exogenous sources. Thus, the recombinant genes of the invention can comprise a coding sequence operably linked to a heterologous genetic element, such as a promoter, enhancer, ribosome binding site, etc. “Heterologous” in this context refers to a genetic element that is not operably linked to the coding sequence in nature. Depending on the cell/vector system utilized, any of a number of suitable transcription and translation control elements, including constitutive and inducible promoters, transcription enhancer elements, transcription terminators, etc. can be used in the expression vector (see e.g., Bitter et al. (1987) Methods in Enzymology, 153:516-544).

Non-limiting examples of suitable promoters for use within a eukaryotic cell are typically viral in origin and include the promoter of the mouse metallothionein I gene (Hamer et al. (1982) J. Mol. Appl. Gen. 1:273); the TK promoter of Herpes virus (McKnight (1982) Cell 31:355); the SV40 early promoter (Benoist et al. (1981) Nature (London) 290:304); the Rous sarcoma virus promoter; the cytomegalovirus promoter (Foecking et al. (1980) Gene 45:101); and the yeast ga14 gene promoter (Johnston et al. (1982) PNAS (USA) 79:6971; Silver et al. (1984) PNAS (USA) 81:5951.

Coding sequences can be operably linked to an inducible promoter. Inducible promoters are those wherein addition of an effector induces expression. Suitable effectors include proteins, metabolites, chemicals, or culture conditions capable of inducing expression.

Alternatively, a coding sequence can be operably linked to a repressible promoter. Repressible promoters are those wherein addition of an effector represses expression.

In some versions, the cell is genetically modified with a recombinant nucleic acid encoding a biosynthetic pathway gene product that is operably linked to a constitutive promoter. Suitable constitutive promoters are known in the art.

Nucleic acids encoding proteins desired to be expressed in a cell may be codon-optimized for that particular type of cell. Codon optimization can be performed for any nucleic acid by “OPTIMUMGENE”-brand gene design system by GenScript (Piscataway, N.J.).

Methods for transforming yeast cells with recombinant DNA and producing polypeptides therefrom are disclosed by Clontech Laboratories, Inc., Palo Alto, Calif., USA (in the product protocol for the “YEASTMAKER”-brand yeast transformation system kit); Reeves et al. (1992) FEMS Microbiology Letters 99:193-198; Manivasakam and Schiestl (1993) Nucleic Acids Research 21(18):4414-5; and Ganeva et al. (1994) FEMS Microbiology Letters 121:159-64. Expression and transformation vectors for transformation into many yeast strains are available. For example, expression vectors have been developed for the following yeasts: Candida albicans (Kurtz, et al. (1986) Mol. Cell. Biol. 6:142); Candida maltosa (Kunze et al. (1985) J. Basic Microbiol. 25:141); Hansenula polymorpha (Gleeson et al. (1986) J. Gen. Microbiol. 132:3459) and Roggenkamp et al. (1986) Mol. Gen. Genet. 202:302); Kluyveromyces fragilis (Das et al. (1984) J. Bacteriol. 158:1165); Kluyveromyces lactis (De Louvencourt et al. (1983) J. Bacteriol. 154:737) and Van den Berg et al. (1990) Bio/Technology 8:135); Pichia quillerimondii (Kunze et al. (1985) J. Basic Microbiol. 25:141); Pichia pastoris (Cregg et al. (1985) Mol. Cell. Biol. 5:3376; U.S. Pat. Nos. 4,837,148; and 4,929,555); Saccharomyces cerevisiae (Hinnen et al. (1978) Proc. Natl. Acad. Sci. USA 75:1929 and Ito et al. (1983) J. Bacteriol. 153:163); Schizosaccharomyces pombe (Beach et al. (1981) Nature 300:706); and Yarrowia lipolytica (Davidow et al. (1985) Curr. Genet. 10:380-471 and Gaillardin et al. (1985) Curr. Genet. 10:49). Genetic transformation systems for metabolic engineering have been developed specifically for a number of yeasts including Mucor circinelloides (Zhang et al. (2007) Microbiology-Sgm 153, 2013-2025), Yarrowia lipolytica (Xuan et al. (1988) Current Genetics 14, 15-21), Rhodotorula glutinis (Li et al. (2012) Appl Microbiol Biotechnol 97(11):4927-36), and Rhodosporidium toruloides (Zhu et al. (2012) Nature Communications, Vol. 3).

An aspect of the invention includes methods of producing a product. The methods in some embodiments comprise culturing a recombinant yeast of the invention in a culture medium for a time sufficient to produce the product. In some versions, the culture medium comprises fatty acids. In some versions, the product comprises one or more of acetone and isopropanol.

The fatty acids in the culture medium can be present in any of a variety of forms. Such forms include free fatty acids, fatty acid esters, fatty acid alcohols, vegetable oil, and other fatty acid-containing feedstocks.

In various versions of the invention, the fatty acids are present in the culture medium in an amount of at least 0.1% v/v, at least 0.5% v/v, at least 1% v/v, at least 2% v/v, at least 3% v/v, at least 4% v/v, at least 5% v/v, at least 6% v/v, at least 7% v/v, at least 8% v/v, at least 9% v/v, at least 10% v/v, at least 15% v/v, at least 20% v/v, at least 25% v/v, at least 30% v/v, at least 35% v/v, at least 40% v/v, or at least 45% v/v. In various versions of the invention, the fatty acids are present in the culture medium in an amount up to 10% v/v, up to 15% v/v, up to 20% v/v, up to 25% v/v, up to 30% v/v, up to 35% v/v, up to 40% v/v, up to 45% v/v, or up to 50% v/v.

In some versions of the invention, the fatty acids comprise free fatty acids present in the culture medium in an amount of at least 0.1% v/v, at least 0.5% v/v, at least 1% v/v, at least 2% v/v, at least 3% v/v, at least 4% v/v, at least 5% v/v, at least 6% v/v, at least 7% v/v, at least 8% v/v, at least 9% v/v, at least 10% v/v, at least 15% v/v, at least 20% v/v, at least 25% v/v, at least 30% v/v, at least 35% v/v, at least 40% v/v, or at least 45% v/v. In some versions of the invention, the fatty acids comprise free fatty acids present in the culture medium in an amount up to 10% v/v, up to 15% v/v, up to 20% v/v, up to 25% v/v, up to 30% v/v, up to 35% v/v, up to 40% v/v, up to 45% v/v, or up to 50% v/v.

In various versions of the invention, the fatty acids are present in the culture medium in an amount of at least 0.1% w/v, at least 0.5% w/v, at least 1% w/v, at least 2% w/v, at least 3% w/v, at least 4% w/v, at least 5% w/v, at least 6% w/v, at least 7% w/v, at least 8% w/v, at least 9% w/v, at least 10% w/v, at least 15% w/v, at least 20% w/v, at least 25% w/v, at least 30% w/v, at least 35% w/v, at least 40% w/v, or at least 45% w/v. In various versions of the invention, the fatty acids are present in the culture medium in an amount up to 10% w/v, up to 15% w/v, up to 20% w/v, up to 25% w/v, up to 30% w/v, up to 35% w/v, up to 40% w/v, up to 45% w/v, or up to 50% w/v.

In some versions of the invention, the fatty acids comprise free fatty acids present in the culture medium in an amount of at least 0.1% w/v, at least 0.5% w/v, at least 1% w/v, at least 2% w/v, at least 3% w/v, at least 4% w/v, at least 5% w/v, at least 6% w/v, at least 7% w/v, at least 8% w/v, at least 9% w/v, at least 10% w/v, at least 15% w/v, at least 20% w/v, at least 25% w/v, at least 30% w/v, at least 35% w/v, at least 40% w/v, or at least 45% w/v. In some versions of the invention, the fatty acids comprise free fatty acids present in the culture medium in an amount up to 10% w/v, up to 15% w/v, up to 20% w/v, up to 25% w/v, up to 30% w/v, up to 35% w/v, up to 40% w/v, up to 45% w/v, or up to 50% w/v.

In addition to the fatty acids, the culture medium can comprise other components suitable for the growth of the yeasts of the invention.

In some versions of the invention, the culture medium is devoid of dextrose or contains less than 50% w/v, such as less than 45% w/v, less than 40% w/v, less than 35% w/v, less than 30% w/v, less than 35% w/v, less than 30% w/v, less than 25% w/v, less than 20% w/v, less than 15% w/v, less than 10% w/v, less than 5% w/v, less than 1% w/v, less than 0.5% w/v, less than 0.1% w/v, less than 0.05% w/v, or less than 0.01% w/v dextrose.

In some versions of the invention, the culture medium is devoid of fermentable sugar or contains less than 50% w/v, such as less than 45% w/v, less than 40% w/v, less than 35% w/v, less than 30% w/v, less than 35% w/v, less than 30% w/v, less than 25% w/v, less than 20% w/v, less than 15% w/v, less than 10% w/v, less than 5% w/v, less than 1% w/v, less than 0.5% w/v, less than 0.1% w/v, less than 0.05% w/v, or less than 0.01% w/v fermentable sugar. Examples of fermentable sugars include adonitol, arabinose, arabitol, ascorbic acid, chitin, cellubiose, dextrose, dulcitol, erythrulose, fructose, fucose, galactose, glucose, gluconate, inositol, lactose, lactulose, lyxose, maltitol, maltose, maltotriose, mannitol, mannose, melezitose, melibiose, palatinose, pentaerythritol, raffinose, rhamnose, ribose, sorbitol, sorbose, starch, sucrose, trehalose, xylitol, xylose, and hydrates thereof, among others.

In certain versions of the invention, the medium is contacted with the recombinant yeasts in a fermentation device. The fermentation can be performed under conditions as described in U.S. Pat. No. 9,957,512 B2. The organics produced by the yeast may be separated or purified from any other component of the spent medium for downstream use in other applications. For example, acetone and isopropanol produced by the yeast may be used as solvent and as chemical intermediates to make other chemicals.

All references to volumes herein are understood to pertain to volumes at 25° C. and 1 ATM.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Genomic DNA Extraction

Yeast cells from a 1.5 ml overnight culture were spun down in a 1.7 ml screw-cap tube and resuspended in 200 μl of 2% Triton X-100, 1% SDS, 100 mM NaCl, 10 mM, 1 mM EDTA pH 8.0. 200 μl of 0.5 mm Zirconia Beads were added with 200 μl of phenol:chloroform:isoamyl alcohol (25:24:1) solution stabilized and saturated with Tris-HCl pH 8.0. The mix was then vortexed for at least 2 min and centrifuged at 13000 rpm for 5 min. The aqueous layer was moved to a new tube, and 200 μl of chloroform was added. The mix was vortexed for 10 see and spun for 13,000 rpm for 1 min. The aqueous layer was removed and placed in a new tube with 1.3 ml of 100% ethanol, and the DNA precipitated at −80° C. for at least 30 min. The DNA was spun down at 13000 rpm for 5 min, and the pellet was washed with 1 ml of 70% ethanol. The DNA pellet was air-dried and resuspended in 400 μl of water.

Transformation of Candida viswanathii 50 ml of YPD media was placed in a 250 ml Erlenmeyer flask and was inoculated with one colony from a freshly streaked plate of the uracil auxotrophic strain and grown overnight at 30° C. with shaking. Cells were collected, spun, and washed with 25 ml of sterile water. Cells were spun down and resuspended in 1 ml of sterile water. Cells were then sedimented again and washed with 1 ml of 100 mM lithium acetate, 10 mM Tris HCl, and 1 mM EDTA pH 8.0. Cells were resuspended in 300 μl of 100 mM lithium acetate, 10 mM Tris HCl, and 1 mM EDTA pH 8.0 and incubated at 30° C. for at least 15 min with shaking. 50 μl of the cells were combined with 5 μl of boiled and quickly cooled sheared salmon sperm DNA (10 mg/ml, Thermo Fisher Scientific, USA) and 10 to 20 μl (1-5 ug) of Pac1 (New England Biolabs) digested plasmid DNA. 300 μl of freshly made 40% polyethylene glycol 3350, 100 mM lithium acetate, 10 mM Tris HCl, and 1 mM EDTA pH 8.0 was added, and the yeast was resuspended by gently tapping the tube. The mix was then incubated at 30° C. for at least 15 min with shaking. Cells were spun down, washed with 1 ml of water, resuspended in 200 μl of water, and plated in ScD-ura plates. Plates were incubated at 30° C. or 32° C. until colonies appear.

### Media Preparation

YPD was made by adding 10 g of yeast extract, 20 g of peptone into 800 ml of water. 20 g of dextrose was added to 200 ml of water. Both solutions were autoclaved separately and combined. YPD agar was made similarly, except that 20 g of agar was added to the yeast extract and peptone solution.

ScD-ura media was made by adding 1.7 g of Yeast Nitrogen Base, 5 g of ammonium sulfate, 2 g of Sc-ura amino acid mix (Sunrise Science Products, USA), and 20 g of dextrose in 1 liter of water. The solution was then filtered sterilized. For ScD-ura plates, 1.7 g of Yeast Nitrogen Base, 5 g of ammonium sulfate, 2 g of Sc-ura amino acid mix (Sunrise Science Products, USA), and 20 g of dextrose was added to 250 ml of water. This solution is filter sterilized and heated to 60° C. and added to a 750 ml agar solution where 20 g of agar was added to 750 ml of water and heat sterilized.

SmP media was 1.7 g Yeast Nitrogen Base, 5 g of ammonium sulfate, 1 g of potassium phosphate monobasic, and 1 g of potassium phosphate dibasic to a final volume of 1 liter and filter sterilized.

### Acetone and Isopropanol Determination

Determination of acetone and isopropanol by GC and GC-MS analysis of by a solid phase micro extraction (SPME) head space method.

### Identification of Gene Targets

Several enzyme genes involved in acetone and isopropanol production from different species were chosen for overexpression, and activity analyses in Candida viswanathii. The genes were codon-optimized by using a proprietary algorithm based on the codon preferences of highly expressed genes in Candida viswanathii, and manually adjusted when necessary. A summary of the genes investigated, their species of origin, and corresponding SEQ ID NOs is provided in Table 1.

TABLE 1
Summary of targeted enzymes.

| | | | |
|---|---|---|---|
| | | Protein | DNA |
| | | SEQ | SEQ |
| Enzyme | Organism of Origin | ID NO | ID NO |

Acetyl-CoA thioesterases (E1 activity)

| | | | |
|---|---|---|---|
| TbACT1 | Trypanosoma brucei brucei | 1 | 4 |
| TbACT1 | Trypanosoma brucei gambiense | 2 | 5 |
| TbACT1 | Trypanosoma brucei equiperdum | 3 | 6 |

Acetyl-CoA C-acetyltransferases (E2 activity)

| | | | |
|---|---|---|---|
| CvERG10 IA | Candida viswanathii | 7 | 11 |
| CvERG10 IB | Candida viswanathii | 8 | 12 |
| Y1ERG10 | Yarrowia lipolytica CLIB122 | 9 | 13 |
| CjERG10 | Cyberlindnera jadinii | 10 | 14 |

Acetoacetyl-CoA transferases (E3 activity)

| | | | |
|---|---|---|---|
| CbCTF1 | Clostridium | 15 | 27 |
| CbCTF1-P* | Clostridium | 16 | 28 |
| CbCTF2 | Clostridium | 17 | 29 |
| CbCTF2-P* | Clostridium | 18 | 30 |
| CsCTF1 | Clostridium | 19 | 31 |
| | saccharobutylicum | | |
| CsCTF2 | Clostridium | 20 | 32 |
| | saccharobutylicum | | |
| CaCTF1 | Clostridium algidicarnis | 21 | 33 |
| CaCTF2 | Clostridium algidicarnis | 22 | 34 |
| CtCTF1 | Clostridium | 23 | 35 |
| | thermoalcaliphilum | | |
| CtCTF2 | Clostridium | 24 | 36 |
| | thermoalcaliphilum | | |
| EcCTF1 | Escherichia coli | 25 | 37 |
| EcCTF2 | Escherichia coli | 26 | 38 |

Acetoacetyl-CoA thioesterases (E4 activity)

| | | | |
|---|---|---|---|
| TbACT1 | Trypanosoma brucei brucei | 1 | 4 |
| TbACT1 | Trypanosoma brucei gambiense | 2 | 5 |
| TbACT1 | Trypanosoma brucei equiperdum | 3 | 6 |
| HiybgC | Haemophilus influenzae | 39 | 51 |
| HhybgC | Haemophilus haemolyticus | 40 | 52 |
| HpybgC | Haemophilus parainfluenzae | 41 | 53 |
| RtybgC | Rodentibacter | 42 | 54 |
| | trehalosifermentans | | |
| RmybgC | Rodentibacter myodis | 43 | 55 |
| RgYBGC | Rodentibacter genomosp. 2 | 44 | 56 |
| BxSrfAD | Bacillus | 45 | 57 |
| BaSrfAD | Bacillus atrophaeus | 46 | 58 |
| BhSrfAD | Bacillus halotolerans | 47 | 59 |
| SmACL | Sinorhizobium meliloti | 48 | 60 |
| ExACL | Ensifer | 49 | 61 |
| SfACL | Sinorhizobium fredii | 50 | 62 |

Acetoacetate decarboxylase (E5 activity)

| | | | |
|---|---|---|---|
| CbADC1 | Clostridium beijerinckii | 63-65 | 83-85 |
| CbADC1-P* | Clostridium beijerinckii | 66 | 86 |
| CxADC1 | Clostridium sp. BL-8 | 67 | 87 |
| CgADC1 | Clostridium gasigenes | 68 | 88 |
| CaADC1 | Clostridium acidisoli | 69 | 89 |
| PxADC1 | Paenibacillus sp. ov031 | 70 | 90 |
| HsADC1 | Heyndrickx sporothermodurans | 71 | 91 |
| VxADC1 | Variovorax sp. KK3 | 72 | 92 |
| CcADC1 | Clostridium cagae | 73 | 93 |
| CpADC1 | Clostridium pasteurianum | 74 | 94 |
| PmADC1 | Priestia megaterium | 75 | 95 |
| BaADC1 | Bacillus acidicola | 76 | 96 |
| PfADC1 | Pelosinus fermentans | 77 | 97 |
| CeADC1 | Clostridium estertheticum | 78-79 | 98-99 |
| BxADC1 | Brevibacterium | 80 | 100 |
| CdADC1 | Clostridium sp DL-VIII | 81 | 101 |
| CsADC1 | Clostridium | 82 | 102 |
| | saccharoperbutylacetonicum | | |

Isopropanol dehydrogenase (E6 activity)

| | | | |
|---|---|---|---|
| CbADH1 | Clostridium beijerinckii | 103 | 110 |
| CbADH1-P* | Clostridium beijerinckii | 104 | 111 |
| NcADH1 | Neurospora crassa | 105 | 112 |
| NcADH1-P* | Neurospora crassa | 106 | 113 |
| CpADH1 | Candida parapsilosis | 107 | 114 |
| CpADH1-P* | Candida parapsilosis | 108 | 115 |
| CvADH5 | Candida viswanathii | 109 | 116 |

*P represents enzymes targeted to the peroxisome by adding an N-terminal or C-terminal signal.

### Construction of a Multi-Copy Integration Vector

pVM plasmids in Table 2 were synthesized by Twist Biosciences (USA).

TABLE 2
pVM plasmids constructed.

| Plasmid | 5′ Addition | Sequence | 3′ Addition | Background |
|---|---|---|---|---|
| pVM1 | None | URA3::(P_{POX4}- | None | pTwist-Chlor- |
| | | T_{POX4}) | | High-Copy |
| | | (SEQ ID NO: 123) | | |
| pVM2 | ACAGCTCTTCC | CbCTF1 | TAAGAGGGAAGAGC | pTwist-Kan- |
| | (SEQ ID NO: 137) | (SEQ ID NO: 27) | TGTACA | High-Copy |
| | | | (SEQ ID NO: 141) | |
| pVM3 | ACAGCTCTTCC | CbCTF1-P | TAAGAGGGAAGAGC | pTwist-Kan- |
| | (SEQ ID NO: 137) | (SEQ ID NO: 28) | TGTACA | High-Copy |
| | | | (SEQ ID NO: 141) | |
| pVM4 | ACAGCTCTTCC | CbCTF2 | TAAGAGGGAAGAGC | pTwist-Kan- |
| | (SEQ ID NO: 137) | (SEQ ID NO: 29) | TGTACA | High-Copy |
| | | | (SEQ ID NO: 141) | |
| pVM5 | ACAGCTCTTCC | CbCTF2-P | TAAGAGGGAAGAGC | pTwist-Kan- |
| | (SEQ ID NO: 137) | (SEQ ID NO: 30) | TGTACA | High-Copy |
| | | | (SEQ ID NO: 141) | |
| pVM6 | ACAGCTCTTCC | CbADC1 | TAAGAGGGAAGAGC | pTwist-Kan- |
| | (SEQ ID NO: 137) | (SEQ ID NO: 83) | TGTACA | High-Copy |
| | | | (SEQ ID NO: 141) | |
| pVM8 | ACAGCTCTTCC | CbADH1-P | TAAGAGGGAAGAGC | pTwist-Kan- |
| | (SEQ ID NO: 137) | (SEQ ID NO: 111) | TGTACA | High-Copy |
| | | | (SEQ ID NO: 141) | |
| pVM9 | ACAGCTCTTCC | NcADH1-P | TAAGAGGGAAGAGC | pTwist-Kan- |
| | (SEQ ID NO: 137) | (SEQ ID NO: 113) | TGTACA | High-Copy |
| | | | (SEQ ID NO: 141) | |
| pVM10 | | CAT2a | | pTwist-Chlor- |
| | | (SEQ ID NO: 130) | | High-Copy |
| pVM11 | | CAT2b | | pTwist-Kan- |
| | | (SEQ ID NO: 131) | | High-Copy |
| pVM12 | | CAT2c | | pTwist-Kan- |
| | | (SEQ ID NO: 132) | | High-Copy |
| pVM13 | ACAGCTCTTCC | CvERG10 | TAAGAGGGAAGAGC | pTwist-Kan- |
| | (SEQ ID NO: 137) | (SEQ ID NO: 11) | TGTACA | High-Copy |
| | | | (SEQ ID NO: 141) | |
| pVM14 | ACAGCTCTTCC | CpADH1-P | TAAGAGGGAAGAGC | pTwist-Kan- |
| | (SEQ ID NO: 137) | (SEQ ID NO: 115) | TGTACA | High-Copy |
| | | | (SEQ ID NO: 141) | |
| pVM16 | ATGGTCTCAG | POX4 Promoter | AGAAGAGCGCTCTTC | pTwist-Amp- |
| | GAG | (SEQ ID NO: 117) | CGCTGTCATGAGACC | High-Copy |
| | (SEQ ID NO: 138) | | AT | |
| | | | (SEQ ID NO: 142) | |
| pVM20 | ATGGTCTCAG | PEX11 Promoter | AGAAGAGCGCTCTTC | pTwist-Amp- |
| | TCA | (SEQ ID NO: 118) | CGCTCGCTTGAGACC | High-Copy |
| | (SEQ ID NO: 139) | | AT | |
| | | | (SEQ ID NO: 143) | |
| pVM21 | AGAGCTCTTC | POX4 Terminator | GCTAGAAGAGCAGA | pTwist-Kan- |
| | CGAG | (SEQ ID NO: 121) | TCGACCCACAGGGA | High-Copy |
| | (SEQ ID NO: 140) | | GGAAGTAAACTGCTA | |
| | | | TTCGTCGCCGATGGT | |
| | | | GGTAACTAATTATGT | |
| | | | TCCTTGCCACTAT | |
| | | | (SEQ ID NO: 144) | |
| pVM22 | AGAGCTCTTC | ETF1 Terminator | GCTAGAAGAGCAGA | pTwist-Kan- |
| | CGAG | (SEQ ID NO: 122) | TCGACCCACAGGGA | High-Copy |
| | (SEQ ID NO: 140) | | GGAAGTAAACTGCTA | |
| | | | TTCGTCGCCGATGGT | |
| | | | GGTAACTAATTATGT | |
| | | | TCCTTGCCACTAT | |
| | | | (SEQ ID NO: 144) | |
| pVM23 | | URA3-A | | pTwist-Kan- |
| | | (SEQ ID NO: 124) | | High-Copy |
| pVM24 | | URA3-B | | pTwist-Kan- |
| | | (SEQ ID NO: 125) | | High-Copy |
| pVM25 | | URA3-C | | pTwist-Amp- |
| | | (SEQ ID NO: 126) | | High-Copy |
| pVM33 | | GRE3-A | | pTwist-Chlor- |
| | | (SEQ ID NO: 127) | | High-Copy |
| pVM34 | | GRE3-B | | pTwist-Kan- |
| | | (SEQ ID NO: 128) | | High-Copy |
| pVM35 | | GRE3-C | | pTwist-Kan- |
| | | (SEQ ID NO: 129) | | High-Copy |
| pVM61 | ATGGTCTCAG | ETF1 Promoter | AGAAGAGCGCTCTTC | pTwist-Amp- |
| | GAG | (SEQ ID NO: 119) | CGCTGTCATGAGACC | High-Copy |
| | (SEQ ID NO: 138) | | AT | |
| | | | (SEQ ID NO: 141) | |
| pVM65 | ACAGCTCTTCC | TbACT1 | TAAGAGGGAAGAGC | pTwist-Kan- |
| | (SEQ ID NO: 137) | (SEQ ID NO: 4) | TGTACA | High-Copy |
| | | | (SEQ ID NO: 141) | |
| pVM67 | ACAGCTCTTCC | CjERG10 | TAAGAGGGAAGAGC | pTwist-Kan- |
| | (SEQ ID NO: 137) | (SEQ ID NO: 14) | TGTACA | High-Copy |
| | | | (SEQ ID NO: 141) | |
| pVM71 | ATGGTCTCAG | TDH3 Promoter | AGAAGAGCGCTCTTC | pTwist-Amp- |
| | TCA | (SEQ ID NO: 120) | CGCTCGCTTGAGACCAT | High-Copy |
| | (SEQ ID NO: 139) | | (SEQ ID NO: 143) | |

### Construction of the pMW Vectors

Cloning was done using a GoldenBraid inspired method (Sarrion-Perdigones et al. 2013). In a 125 μl PCR tube, 5 ng of the receipt vector and 3× times equimolar of the one, two plasmids, two annealed oligos, or PCR fragment that contain the inserts were combined. One μl of T4 DNA Ligase Buffer (New England Biolabs), 0.5 μl of T4 DNA Ligase (New England Biolabs), 0.5 μl of Bsal or SapI enzyme (New England Biolabs), and water was added to a final volume of 10 μl. The reaction was then placed in a thermocycler and subjected to the following program:
1 cycle at 37° C. for 30 see
25 cycles of 37° ° C. for 2 min followed by 16° C. for 5 minutes
1 cycle of 37° ° C. for 10 min
1 cycle of 80° C. for 5 min

1 μl of the reaction was then transformed into DH5alpha cells (Monserate Biotechnology Group, USA) as recommended by the manufacturing and plated in selective media. The correct final construct was verified by restriction enzyme analysis. See Table 3 for specific inserts used for each pMW plasmid. o56/o57 means an annealed mixture of oligos o56 and o57.

TABLE 3
Specific inserts used for pMW plasmids.

| Plasmid | Recipient | Insert 1 | Insert 2 | Enzyme | Selection |
|---|---|---|---|---|---|
| pMW1 | pVM23 | pVM25 | | BsaI | Kanamycin |
| pMW2 | pVM24 | pVM25 | | BsaI | Kanamycin |
| pMW4 | pVM16 | pVM2 | pVM22 | SapI | Ampicillin |
| pMW6 | pVM16 | pVM3 | pVM22 | SapI | Ampicillin |
| pMW7 | pVM20 | pVM4 | pVM21 | SapI | Ampicillin |
| pMW8 | pVM20 | pVM5 | pVM21 | SapI | Ampicillin |
| pMW9 | pVM10 | pMW1 | pVM11 | SapI | Chloramphenicol |
| pMW10 | pVM10 | pMW2 | pVM12 | SapI | Chloramphenicol |
| pMW11 | pVM1 | pMW4 | pMW7 | BsaI | Chloramphenicol |
| pMW12 | pVM1 | pMW6 | pMW8 | BsaI | Chloramphenicol |
| pMW13 | pVM1 | pVM6 | | SapI | Chloramphenicol |
| pMW18 | pVM20 | pVM6 | pVM21 | SapI | Ampicillin |
| pMW19 | pVM16 | pVM13 | pVM22 | SapI | Ampicillin |
| pMW21 | pVM1 | pVM8 | | SapI | Chloramphenicol |
| pMW22 | pVM1 | pVM9 | | SapI | Chloramphenicol |
| pMW23 | pVM1 | pVM14 | | SapI | Chloramphenicol |
| pMW24 | pVM1 | pMW19 | pMW17 | BsaI | Chloramphenicol |
| pMW25 | pVM1 | pMW20 | pMW18 | BsaI | Chloramphenicol |
| pMW29 | pVM61 | pVM2 | pVM21 | SapI | Ampicillin |
| pMW30 | pVM61 | pVM6 | pVM21 | SapI | Ampicillin |
| pMW31 | pVM1 | pVM13 | | SapI | Chloramphenicol |
| pMW38 | pVM1 | pMW35 | o56/o57 | BsaI | Chloramphenicol |
| pMW39 | pVM1 | pMW36 | o56/o57 | BsaI | Chloramphenicol |
| pMW40 | pVM1 | pMW37 | o56/o57 | BsaI | Chloramphenicol |
| pMW48 | pVM71 | pVM4 | pVM22 | SapI | Ampicillin |
| pMW52 | pVM61 | pVM65 | pVM21 | SapI | Ampicillin |
| pMW54 | pVM1 | pMW29 | pMW48 | BsaI | Chloramphenicol |
| pMW55 | pVM1 | pMW30 | pMW50 | BsaI | Chloramphenicol |
| pMW58 | pVM1 | pMW52 | o56/o57 | BsaI | Chloramphenicol |
| pMW63 | pVM33 | pMW1 | pVM34 | SapI | Chloramphenicol |
| pMW64 | pVM33 | pMW2 | pVM35 | SapI | Chloramphenicol |

The following pMW plasmids had at least one insert from a PCR product.

The CBT1 open reading frame of pVM6 to make pMW14 and pMW17 was amplified with oligos o1 and o3 using Q5 High-Fidelity 2X Master Mix (New England Biolabs, USA) as recommended by the manufacturer. The PCR fragment was purified using DNA Clean & Concentrator-5, Capped Columns, as recommended by the Manufacturer (Zymo Research, USA). The concentration was determined using the DS DNA Broad Range kit by Denovix (USA) as recommended.

The PCR product then was used as an insert in a GoldenBraid ligation as described above with the other parts shown in the table below to make pMW14 and pMW17. Correct final construct was verified by restriction enzyme analysis and sequence verified of the amplified open reading frame.

In a similar manner as CBT1 open reading frame, CjERG10 open reading frame of pVM13 was amplified to make pMW20 was amplified with oligos o1 and o36, the CbADH1 open reading frame from pVM8 was amplified to make pMW26 and pMW35 using oligos o1 and o34, the NcADHI open reading frame from pVM9 was amplified to make pMW27 and pMW36 using oligos o1 and o35, and CpADH1 open reading frame from pVM14 was amplified to make pMW28 and pMW61 using oligos o1 and o37.

Table 4 shows specific inserts used for the pMW plasmids.

TABLE 4
Specific inserts used for pMW plasmids.

| Plasmid | Recipient | Insert 1 | Insert 2 | Enzyme | Selection |
|---|---|---|---|---|---|
| pMW14 | pVM1 | PCR o1- | None | SapI | Chloramphenicol |
| | | o3 (pVM6) | | | |
| pMW17 | pVM20 | PCR o1- | pVM21 | SapI | Ampicillin |
| | | o3(pVM6) | | | |
| pMW19 | pVM16 | pVM13 | pVM22 | SapI | Ampicillin |
| pMW20 | pVM16 | PCR o1- | pVM22 | SapI | Ampicillin |
| | | o36(pVM13) | | | |
| pMW26 | pVM1 | PCR o1- | None | SapI | Chloramphenicol |
| | | o34(pVM8) | | | |
| pMW27 | pVM1 | PCR o1- | None | SapI | Chloramphenicol |
| | | o35(pVM9) | | | |
| pMW28 | pVM1 | PCR o1- | None | SapI | Chloramphenicol |
| | | o37(pVM14) | | | |
| pMW35 | pVM61 | PCR o1- | pVM21 | SapI | Ampicillin |
| | | o34(pVM8) | | | |
| pMW36 | pVM61 | PCR o1- | pVM21 | SapI | Ampicillin |
| | | o35(pVM9) | | | |
| pMW37 | pVM61 | PCR o1- | pVM21 | SapI | Ampicillin |
| | | o37(pVM14) | | | |
| pMW50 | pVM71 | PCR o1- | pVM22 | SapI | Ampicillin |
| | | o34(pVM8) | | | |

### Construction of a Cat2 Deletion Strain

ATCC20913 (American Type Culture Collection, USA) was transformed with a PacI digested pMW9 and plated in ScD-ura plate. URA+ colonies were restreaked, and single colonies were used to start a 5 ml YPD overnight at 30° C. Genomic DNA was extracted, and deletion of the first allele of CAT1 was verified by PCR using Apex Taq RED Master Mix (Genesee Scientific) as recommended and using oligos o8 and o11, and oligos o9 and o10. A strain with the correct amplification bands was named YU10.

100 μl of the overnight of YU10 was plated in ScD+5-FOA plates and incubated at 30° C. for a couple of days. 5-FOA resistant colonies were restreaked in ScD+5-FOA plates, and single colonies were used to start a 5 ml YPD overnight. Genomic DNA was extracted, and the loop out of the URA3 in the CAT1 loci was verified by PCR using Apex Taq RED Master Mix (Genesee Scientific) as recommended and using oligos o8 and o9. A strain with the correct amplification bands was named YU14.

YU14 was transformed with a PacI digested pMW10 and plated in ScD-ura plate. URA+ colonies were restreaked, and single colonies were used to start a 5 ml YPD overnight at 30° C. Genomic DNA was extracted, and the deletion of the second allele of CAT2 was verified by PCR using Apex Taq RED Master Mix (Genesee Scientific) as recommended and using oligos o12, o13, o14, and o15. Oligos o12 and o13 amplifies an 803 bp nucleotide fragment of the actin gene, while o14 and o15 amplifies a 486 bp nucleotide fragment of the CAT2 gene. A strain with the correct amplification bands was named YU18 and YU19.

100 μl of the overnight of YU18 was plated in ScD+5-FOA plates and incubated at 30° C. for a couple of days. 5-FOA resistant colonies were restreaked in ScD+5-FOA plates, and single colonies were used to start a 5 ml YPD overnight. Genomic DNA was extracted, and the loop out of the URA3 in the CAT2 loci was verified by PCR using Apex Taq RED Master Mix (Genesee Scientific) as recommended and using oligos o8 and o9. A strain with the correct amplification bands was named YU20 and YU21.

### Verification of the Integration of Specific Genes

A four oligo PCR method was developed. It used amplification of an 803 bp fragment of the actin gene as a positive control and amplification of the gene of interest by two gene-specific oligos that resulted in a smaller amplicon. Apex Taq RED Master Mix (Genesee Scientific, USA) was used as recommended by the manufacturer. Table 5 shows the oligos used for each open reading frame. Table 6 shows the sequences of the oligos used in the present examples.

TABLE 5
Oligos used for open reading frame amplification.

| Gene | Oligo 1 | Oligo 2 | Fragment size (nt) |
|---|---|---|---|
| Actin | o12 | o13 | 803 |
| CbCTF1 | o16 | o17 | 486 |
| CbCTF2 | o18 | o19 | 487 |
| CbADC1 | o20 | o21 | 500 |
| CbCTF1-P | o22 | o23 | 501 |
| CbCTF2-P | o18 | o24 | 526 |
| CbADC1p | o20 | o25 | 600 |
| CbADH1 | o38 | o39 | 566 |
| NcADH1 | o40 | o41 | 533 |
| CvERG10 | o42 | o43 | 552 |
| CpADH1 | o44 | o45 | 536 |
| TbACT1 | o106 | o107 | 485 |

TABLE 6
Sequence of oligos used in the present examples.

| | | SEQ |
|---|---|---|
| Name | Sequence | ID NO |
| o1 | GGAAGTGCCATTCCGCCTGACCT | 145 |
| o3 | TGTACAGCTCTTCCCTCTTATCAAAGCTTTGCTCGTCTA | 146 |
| | CCCTTAACTGACAAGTAATCATGAACAACCTTTGGTG | |
| o8 | CCTTCGGAGTTTCGTATCCTTATCACC | 147 |
| o9 | CTTTTATTGTATCGTTGTTTCTGTGTGCATAACAC | 148 |
| o1o | GGATCGCAAGCAACGAGAACGCC | 149 |
| o11 | GGCGTTCTCGTTGCTTGCGATCC | 150 |
| o12 | CAAGAGCTGTTTTCCCATCTATCGTTGG | 151 |
| o13 | GTATAATTCTTTTCTGACATCCAAGTCACACTTC | 152 |
| o14 | GTTGTCACAACAAGTATTAAAGAATTCAACCAAATCC | 153 |
| o15 | CTGGGTCCAAGCTTTCATCCAAAACC | 154 |
| o16 | GATTTGAAAACTTGAGATCCTTCTTTAAGGATGG | 155 |
| o17 | CCCTTGTAAAATGTGTTTCCTGCTTCATCAAC | 156 |
| o18 | CAGAAAACGGAATTGTTGGAATGGGAGC | 157 |
| o19 | CCTCAATTCGTTTGAAATCAACAAATCTGCTGC | 158 |
| o20 | GGATAGAGCATACGTTAGATTTGAAATGATGGC | 159 |
| o21 | CCAAAACTGGCAAATCAGCCAATGGTG | 160 |
| o22 | CACAGGAATTGGAAAGTTGATTTCAAACAACCAAG | 161 |
| o23 | CAAAGCTTTGCTCGTCTACCTGCTGG | 162 |
| o24 | GCTCGTCTACCAACTGCCATTGGC | 163 |
| o25 | CAAAGCTTTGCTCGTCTACCCTTAACTG | 164 |
| o34 | TGTACAGCTCTTCCCTCTTACAAAATAACAACTGCCTTA | 165 |
| | ATCAAATCCTTGGG | |
| o35 | TGTACAGCTCTTCCCTCTTACTTTGATGTATCAACAACG | 166 |
| | TATCGTCCAACAATC | |
| o36 | TGTACAGCTCTTCCCTCTTAATCTGCATCAATCTTTTCA | 167 |
| | ATAACAACTGCTGATGC | |
| o37 | TGTACAGCTCTTCCCTCTTATGGGTTAAAAACAACTCTT | 168 |
| | CCTTCGTATGC | |
| o38 | GAAGCAGTTGGAGAAGTTGTTGAAGTTGG | 169 |
| o39 | CGATCAACTCCCTTTCCGTTTGTCAAC | 170 |
| o4o | CTCAGGAGTTTGTCATACAGATTTGCATGC | 171 |
| o41 | CCTTTTCTTCTCCTCCATCAATTGCAATTGC | 172 |
| o42 | GCAAGCAAACGTTGGACAAGCACCAG | 173 |
| o43 | GACTTCAATCTTTCTTCGTTAAACTTTCCAATTTCTTC | 174 |
| o44 | GAGAAACGATTTGCCAGTTCATAAGCCAAAG | 175 |
| o45 | CTTGAATTGCGTTTCCTCCCAATCCTC | 176 |
| o56 | ATGGTCTCAGTCAAGTAGAGCGATCTTCCGCTCGCTTGA | 177 |
| | GACCAT | |
| o57 | ATGGTCTCAAGCGAGCGGAAGATCGCTCTACTTGACTGA | 178 |
| | GACCAT | |
| o106 | GATCTCGTTGGGAAGGAATTATGGAGTG | 179 |
| o107 | CACTCGACGTCTGAGCACGTTATGG | 180 |
| oF1 | CAGGAGACAAGATCATTCGCTGCGG | 181 |
| oF2 | GCTCGGCGTGTTGTGGGCG | 182 |
| OF3 | GGGAAATATTGTGCGGCAGCCGC | 183 |
| oF4 | CTACAGAACCGACATTCCTGTCCGC | 184 |
| oF5 | GGGACCTTAACCAAGTTTCTTGGTCTTC | 185 |

### Construction of Strains YU3, YU4, YU5, YU6, YU8, and YU9

YU1 was transformed with Pac1 digested pVM1, pMW11+pMW13, or pMW12+pMW14 plasmids and plated in ScD-ura plate. URA+colonies were restreaked, and single colonies were used to start a 5 ml ScD-ura overnight at 30° C. Genomic DNA was extracted, and the integration was verified with the PCR method described above.

Characteristics of the resulting strains are shown in Table 7.

TABLE 7
Strains YU3, YU4, YU5, YU6, YU8, and YU9.

| | | Parental | Plasmid |
|---|---|---|---|
| Strain | Genotype | Strain | Transformed |
| YU3 | ura3/ura3 + 3′URA3-P.POX4-T.POX4- | YU1 | pVM1 |
| | 5′URA3 | | |
| YU4 | ura3/ura3 + 3′URA3-P.POX4-T.POX4- | YU1 | pVM1 |
| | 5′URA3 | | |
| YU5 | 3′ URA3-P.POX4-CbCTF1-T.ETF1- | YU1 | pMW11 + |
| | P.PEX11-CbCTF2-T.POX4-5′ URA3 + | | pMW13 |
| | 3′ URA3-P.POX4-CbADC1-T.POX4-5′ | | |
| | URA3 ura3/ura3 | | |
| YU6 | 3′ URA3-P.POX4-CbCTF1-T.ETF1- | YU1 | pMW11 + |
| | P.PEX11-CbCTF2-T.POX4-5′ URA3 + | | pMW13 |
| | 3′ URA3-P.POX4-CbADC1-T.POX4-5′ | | |
| | URA3 ura3/ura3 | | |
| YU7 | 3′ URA3-P.POX4-CbCTF1-T.ETF1- | YU1 | pMW11 + |
| | P.PEX11-CbCTF2-T.POX4-5′ URA3 + | | pMW13 |
| | 3′ URA3-P.POX4-CbADC1-T.POX4-5′ | | |
| | URA3 ura3/ura3 | | |
| YU8 | 3′ URA3-P.POX4-CbCTF1-P-T.ETF1- | YU1 | pMW12 + |
| | P.PEX11-CbCTF2-P-T.POX4-5′ URA3 + | | pMW14 |
| | 3′ URA3-P.POX4-CbADC1-P-T.POX4-5′ | | |
| | URA3 ura3/ura3 | | |
| YU9 | 3′ URA3-P.POX4-CbCTF1-P-T.ETF1- | YU1 | pMW12 + |
| | P.PEX11-CbCTF2-P-T.POX4-5′ URA3 + | | pMW14 |
| | 3′ URA3-P.POX4-CbADC1-P-T.POX4-5′ | | |
| | URA3 ura3/ura3 | | |

### Fermentation of YU3, YU5, YU6, YU8, and YU9

Overnights of YU3, YU5, YU6, YU8, and YU9 in 3 ml of ScD-ura media were started from a single colony from freshly streaked ScD-ura plates and incubated at 30° C. overnight. 1 ml was used to inoculate 30 ml of YPD in a 250 ml flask and shaken for 24 hours at 30° C.

Cultures were spun down, and the cells resuspended in 15 ml of SmP media. The culture was then transferred to a deep baffled 250 ml Erlenmeyer flask. 670 μl of oleic acid and 70 μl of a 50% glycerol solution were added and incubated for 48 hours at 30° C. Samples were taken, and the amount of acetone was determined by GC.

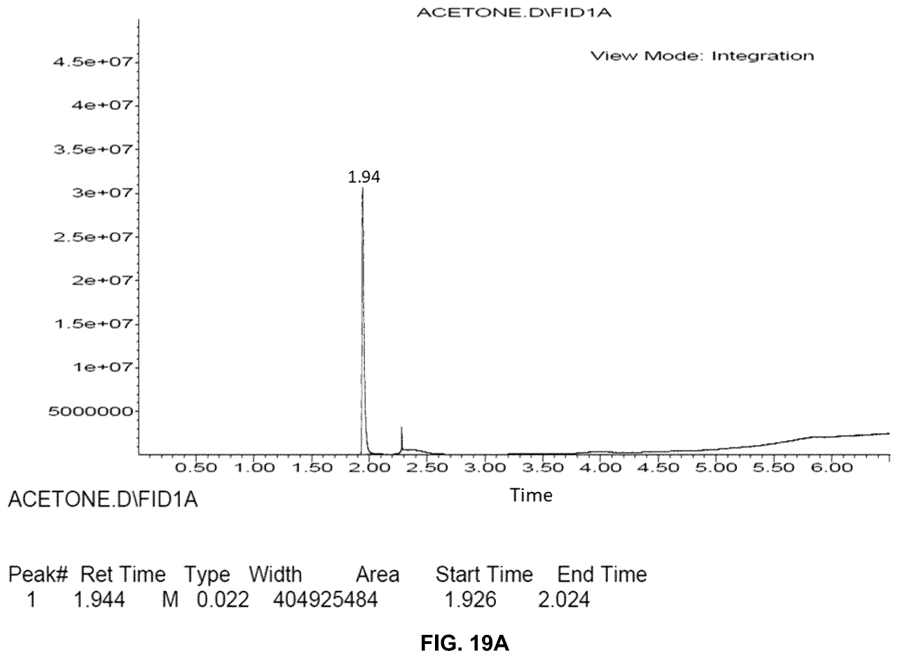

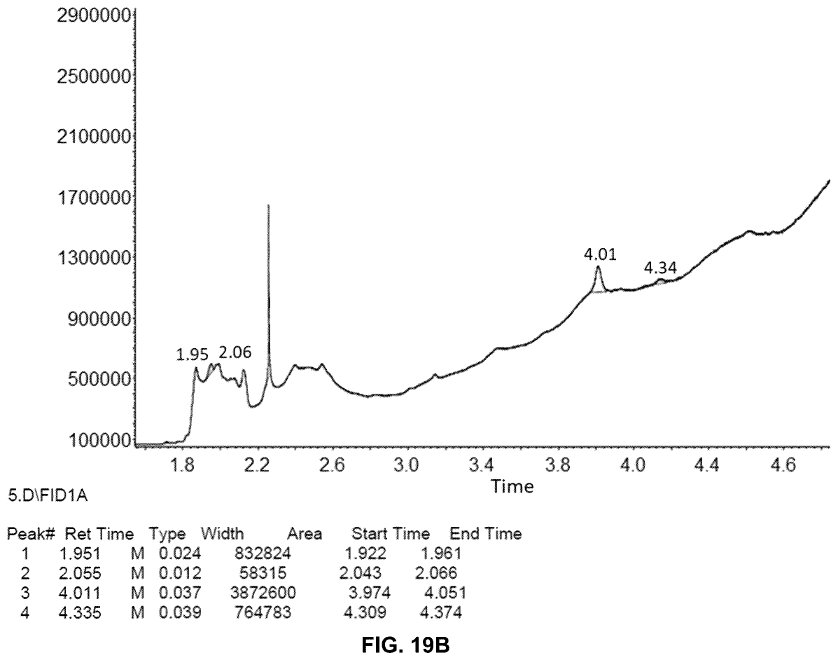

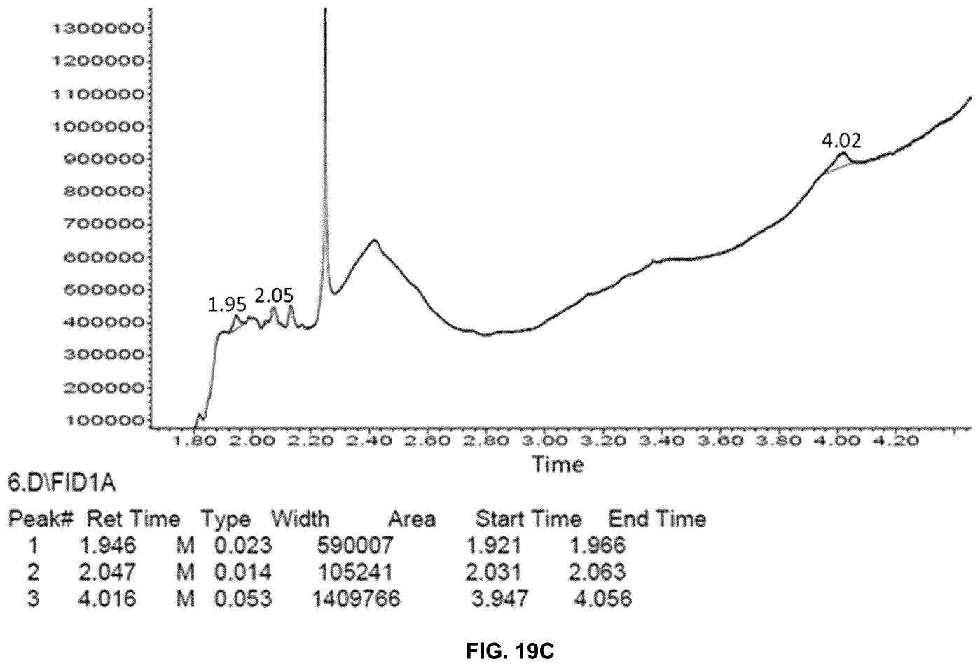

YU5 and YU6 had a small amount of acetone as determined by GC (FIGS. 19A-19C). GC-MS analysis verified the presence of acetone in YU5. These data demonstrated that CbCTF1, CbCTF2, and CbADC1 were active in the yeast and can produce acetone from fatty acids.

Construction of Strains YU32, YU33, YU34, YU35, YU36, YU38, YU39, YU42, and YU43

YU1 was transformed with Pac1 digested of either pMW11+pMW25, or pMW11+pMW25+pMW26 and plated in ScD-ura plate. URA+colonies were restreaked, and single colonies were used to start a 5 ml ScD-ura overnight at 30° ° C. Genomic DNA was extracted, and the integration was verified with the PCR method described above.

YU20 was transformed with Pac1 digested of either pMW12+pMW24, or pMW12+pMW21+pMW24 and plated in ScD-ura plate. URA+colonies were restreaked, and single colonies were used to start a 5 ml ScD-ura overnight at 30° ° C. Genomic DNA was extracted, and the integration was verified with the PCR method described above.

Characteristics of the resulting strains are shown in Table 8.

TABLE 8
Strains YU32, YU33, YU34, YU35,
YU36, YU38, YU39, YU42, and YU43.

| | | Parental | Plasmid |
|---|---|---|---|
| Strain | Genotype | Strain | Transformed |
| YU32 | 3′ URA3-P.POX4-CbCTF1-T.ETF1- | YU1 | pMW11 + |
| | P.PEX11-CbCTF2-T.POX4-5′ URA3 + | | pMW25 |
| | 3′ URA3-P.POX4-CvERG10ΔPTS1- | | |
| | T.ETF1-P.PEX11-CbADC1-T.POX4-5′ | | |
| | URA3 + ura3/ura3 | | |
| YU33 | 3′ URA3-P.POX4-CbCTF1-T.ETF1- | YU1 | pMW11 + |
| | P.PEX11-CbCTF2-T.POX4-5′ URA3 + | | pMW25 |
| | 3′ URA3-P.POX4-CvERG10ΔPTS1- | | |
| | T.ETF1-P.PEX11-CbADC1-T.POX4-5′ | | |
| | URA3 + ura3/ura3 | | |
| YU34 | 3′ URA3-P.POX4-CbCTF1-P-T.ETF1- | YU20 | pMW12 + |
| | P.PEX11-CbCTF2-P-T.POX4-5′ | | pMW24 |
| | URA3 + 3′ URA3-P.POX4-CvERG10- | | |
| | T.ETF1-P.PEX11-CbADC1-P-T.POX4- | | |
| | 5′ URA3 + cat2/cat2 ura3/ura3 | | |
| YU35 | 3′ URA3-P.POX4-CbCTF1-P-T.ETF1- | YU20 | pMW12 + |
| | P.PEX11-CbCTF2-P-T.POX4-5′ | | pMW24 |
| | URA3 + 3′ URA3-P.POX4-CvERG10- | | |
| | T.ETF1-P.PEX11-CbADC1-P-T.POX4- | | |
| | 5′ URA3 + cat2/cat2 ura3/ura3 | | |
| YU37 | 3′ URA3-P.POX4-CbCTF1-P-T.ETF1- | YU20 | pMW12 + |
| | P.PEX11-CbCTF2-P-T.POX4-5′ | | pMW24 + |
| | URA3 + 3′ URA3-P.POX4-CvERG10- | | pMW21 |
| | T.ETF1-P.PEX11-CbADC1-P-T.POX4- | | |
| | 5′ URA3 + 3′ URA3-P.POX4- | | |
| | CbADH1-P-T.POX4-5′ URA3 cat2/ | | |
| | cat2 ura3/ura3 | | |
| YU39 | 3′ URA3-P.POX4-CbCTF1-P-T.ETF1- | YU20 | pMW12 + |
| | P.PEX11-CbCTF2-P-T.POX4-5′ | | pMW24 + |
| | URA3 + 3′ URA3-P.POX4-CvERG10- | | pMW21 |
| | T.ETF1-P.PEX11-CbADC1-P-T.POX4- | | |
| | 5′ URA3 + 3′ URA3-P.POX4- | | |
| | CbADH1-P-T.POX4-5′ URA3 cat2/ | | |
| | cat2 ura3/ura3 | | |
| YU42 | 3′ URA3-P.POX4-CbCTF1-T.ETF1- | YU1 | pMW11 + |
| | P.PEX11-CbCTF2-T.POX4-5′ | | pMW26 + |
| | URA3 + 3′ URA3-P.POX4- | | pMW25 |
| | CvERG10ΔPTS1-T.ETF1-P.PEX11- | | |
| | CbADC1-T.POX4-5′ URA3 + | | |
| | 3′ URA3-P.POX4-CbADH1-T.POX4- | | |
| | 5′ URA3 ura3/ura3 | | |
| YU43 | 3′ URA3-P.POX4-CbCTF1-T.ETF1- | YU1 | pMW11 + |
| | P.PEX11-CbCTF2-T.POX4-5′ URA3 + | | pMW26 + |
| | 3′ URA3-P.POX4-CvERG10ΔPTS1- | | pMW25 |
| | T.ETF1-P.PEX11-CbADC1-T.POX4-5′ | | |
| | URA3 + 3′ URA3-P.POX4-CbADH1- | | |
| | T.POX4-5′ URA3 ura3/ura3 | | |

### Fermentation of YU3, YU32, YU33, YU34, YU35, YU37, YU39, YU42, and YU43

Overnight of YU3, YU18, YU32, YU33, YU34, YU35, YU37, YU39, YU42, and YU43 in 3 ml of ScD-ura media was started from a single colony from freshly streaked ScD-ura plates and incubated at 30° ° C. overnight. 1 ml was used to inoculate 30 ml of YPD in a 250 ml flask and shake for 24 hours at 30° C.

Cultures were spun down, and the cells resuspended in 15 ml of SmP media. The culture was then transferred to a deep baffled 250 ml flask. 670 μl of oleic acid and 70 μl of a 50% glycerol solution were added and incubated for 48 hours at 30° C. Samples were taken, and the amount of acetone was determined by GC.

### Construction of Strains YU22, YU23, YU24, YU25, YU26, YU27, YU28, YU29, YU30, YU31, YU40, and YU41

YU1 was transformed with Pac1 digested pMW21, pMW22, pMW23, pMW26, pMW27, or pMW28 plasmid. URA+colonies were restreaked, and single colonies were used to start a 5 ml ScD-ura overnight at 30° C. Genomic DNA was extracted, and integration was verified with the PCR method described above.

Characteristics of the resulting strains are shown in Table 9.

TABLE 9
Strains YU22, YU23, YU24, YU25, YU26, YU27,
YU28, YU29, YU30, YU31, YU40, and YU41.

| | | Parental | |
|---|---|---|---|
| Strain | Genotype | Strain | Plasmid |
| YU22 | 3′ URA3-P.POX4-CbADH1-P-T.POX4-5′ | YU1 | pMW21 |
| | URA3 ura3/ura3 | | |
| YU23 | 3′ URA3-P.POX4-CbADH1-P-T.POX4-5′ | YU1 | pMW21 |
| | URA3 ura3/ura3 | | |
| YU24 | 3′ URA3-P.POX4-NcADH1-P-T.POX4-5′ | YU1 | pMW22 |
| | URA3 ura3/ura3 | | |
| YU25 | 3′ URA3-P.POX4-NcADH1-P-T.POX4-5′ | YU1 | pMW22 |
| | URA3 ura3/ura3 | | |
| YU26 | 3′ URA3-P.POX4-CpADH1-P-T.POX4-5′ | YU1 | pMW23 |
| | URA3 ura3/ura3 | | |
| YU27 | 3′ URA3-P.POX4-CpADH1-P-T.POX4-5′ | YU1 | pMW23 |
| | URA3 ura3/ura3 | | |
| YU28 | 3′ URA3-P.POX4-NcADH1-T.POX4-5′ | YU1 | pMW27 |
| | URA3 ura3/ura3 | | |
| YU29 | 3′ URA3-P.POX4-NcADH1-T.POX4-5′ | YU1 | pMW27 |
| | URA3 ura3/ura3 | | |
| YU30 | 3′ URA3-P.POX4-CpADH1-T.POX4-5′ | YU1 | pMW28 |
| | URA3 ura3/ura3 | | |
| YU31 | 3′ URA3-P.POX4-CpADH1-T.POX4-5′ | YU1 | pMW28 |
| | URA3 ura3/ura3 | | |
| YU40 | 3′ URA3-P.POX4-CbADH1-T.POX4-5′ | YU1 | pMW26 |
| | URA3 ura3/ura3 | | |
| YU41 | 3′ URA3-P.POX4-CbADH1-T.POX4-5′ | YU1 | pMW26 |
| | URA3 ura3/ura3 | | |

### Fermentation of Strains YU3, YU22, YU23, YU24, YU25, YU26, YU27, YU28, YU29, YU30, YU31, YU40 and YU41

Overnight of YU3, YU22, YU23, YU24, YU25, YU26, YU27, YU28, YU29, YU30, YU31, YU40, and YU41 in 3 ml of ScD-ura media was started from a single colony of freshly streaked ScD-ura plate and incubated at 32° C. overnight. 3 ml was used to inoculate 30 ml of YPD in a 250 ml flask and incubated for 24 hours at 32° C.

Cultures were spun down, and the cells resuspended in 15 ml of SmP media. The culture was then transferred to a deep baffled 250 ml flask. 670 μl of oleic acid and 70 μl of a 50% glycerol solution were added and incubated for 6 hours at 32° C. After six hours, 150 μl of acetone was added and incubated for 24 hours. Samples were taken, and the amount of acetone was determined by GC.

### Construction of Strains YU45, YU50, YU53, YU54, and YU55

YU20 was transformed with Pac1 digested either pMW12 or pMW12+pMW31 plasmids and plated in ScD-ura plate. URA+colonies were restreaked, and single colonies were used to start a 5 ml ScD-ura overnight at 30° ° C. Genomic DNA was extracted, and the integration was verified with the PCR method described above.

Characteristics of the resulting strains are shown in Table 10.

TABLE 10
Strains YU45, YU50, YU53, YU54, and YU55.

| | | Parental | |
|---|---|---|---|
| Strain | Genotype | Strain | Plasmid |
| YU45 | 3′ URA3-P.POX4-CbCTF1-P-TETF1- | YU20 | pMW12 |
| | P.PEX11-CbCTF2-P-T.POX4-5′ | | |
| | URA3 + cat2/cat2 ura3/ura3 | | |
| YU50 | 3′ URA3-P.POX4-CbCTF1-P-T.ETF1- | YU20 | pMW12 |
| | P.PEX11-CbCTF2-P-T.POX4-5′ | | |
| | URA3 + cat2/cat2 ura3/ura3 | | |
| YU53 | 3′ URA3-P.POX4-CbCTF1-P-T.ETF1- | YU20 | pMW12 + |
| | P.PEX11-CbCTF2-P-T.POX4-5′ URA3 + | | pMW31 |
| | 3′ URA3-P.POX4-CvERG10-T.POX4-5′ | | |
| | URA3 + cat2/cat2 ura3/ura3 | | |
| YU54 | 3′ URA3-P.POX4-CbCTF1-P-T.ETF1- | YU20 | pMW12 + |
| | P.PEX11-CbCTF2-P-T.POX4-5′ URA3 + | | pMW31 |
| | 3′ URA3-P.POX4-CvERG10-T.POX4-5′ | | |
| | URA3 + cat2/cat2 ura3/ura3 | | |
| YU55 | 3′ URA3-P.POX4-CbCTF1-P-T.ETF1- | YU20 | pMW12 + |
| | P.PEX11-CbCTF2-P-T.POX4-5′ URA3 + | | pMW31 |
| | 3′ URA3-P.POX4-CvERG10-T.POX4-5′ | | |
| | URA3 + cat2/cat2 ura3/ura3 | | |

### Fermentation of YU3, YU4, YU18, YU19 YU45, YU50, YU53, YU54, and YU55

Overnight of YU3, YU4, YU18, YU19, YU45, YU50, YU53, YU54, and YU55 in 3 ml of ScD-ura media was started from a single colony of freshly streaked ScD-ura plate and incubated at 30° ° C. overnight. 3 ml was used to inoculate 30 ml of YPD in a 250 ml flask and shake for 24 hours at 30° C.

Cultures were spun down, and the cells resuspended in 15 ml of SmP media. The culture was then transferred to a deep baffled 250 ml flask. 670 μl of oleic acid and 70 μl of a 50% glycerol solution were added and incubated for 48 hours at 30° C. 1.5 ml samples were taken and placed in a 1.7 ml microcentrifuge tube. Cells were spun down and the supernatant filtered through a 0.22 um PTFE filter. Samples were diluted 10-fold with water and acetate and acetoacetate determined using an acetate colorimetric assay (Biovision, USA) or acetoacetate colorimetric assay (Biovision, USA), as recommended by the manufacturer. No apparent increase of acetic acid and acetoacetate was observed in any samples. Results are shown in Table 11.

TABLE 11
Acetate and acetoacetate concentrations in fermented cultures.

Concentration mg/L

| | Acetate | Acetoacetate |
|---|---|---|
| YU3 | −9.1 | 226.7 |
| YU4 | −6.9 | 292.6 |
| YU18 | 2.9 | 384.8 |
| YU19 | 7.3 | 42.3 |
| YU45 | 9.5 | 108.2 |
| YU50 | 13.9 | 424.3 |
| YU53 | 24.8 | 239.9 |
| YU54 | 7.3 | 700.9 |
| YU55 | −4.7 | 266.2 |

### Construction of Strains YU72, YU73, YU74, YU75, YU76, and YU77

YU1 was transformed with Pac1 digested of either pMW54+pMW55, or pMW55+pMW58 plasmids and plated in ScD-ura plate. URA+colonies were restreaked and single colonies used to start a 5 ml ScD-ura overnight at 30° ° C. Genomic DNA was extracted and verification of the integration was performed with the PCR method described above.

Characteristics of the resulting strains are shown in Table 12.

TABLE 12
Strains YU72, YU73, YU74, YU75, YU76, and YU77.

| | | Parental | |
|---|---|---|---|
| Strain | Genotype | Strain | Plasmid |
| YU72 | 3′ URA3 -P.ETF1-CbCTF1-T.POX4 + | YU1 | pMW54 + |
| | P.TDH3-CbCTF2-T.ETF1- 5′ URA3 3′ | | pMW55 |
| | URA3 -P.ETF1-CbADC1-T.POX4 + | | |
| | P.TDH3-CbADH1-T.ETF1- 5′ URA3 | | |
| | ura3/ura3 | | |
| YU73 | 3′ URA3 -P.ETF1-CbCTF1-T.POX4 + | YU1 | pMW54 + |
| | P.TDH3-CbCTF2-T.ETF1- 5′ URA3 3′ | | pMW55 |
| | URA3 -P.ETF1-CbADC1-T.POX4 + | | |
| | P.TDH3-CbADH1-T.ETF1- 5′ URA3 | | |
| | ura3/ura3 | | |
| YU74 | 3′ URA3 -P.ETF1-CbCTF1-T.POX4 + | YU1 | pMW54 + |
| | P.TDH3-CbCTF2-T.ETF1- 5′ URA3 3′ | | pMW55 |
| | URA3 -P.ETF1-CbADC1-T.POX4 + | | |
| | P.TDH3-CbADH1-T.ETF1- 5′ URA3 | | |
| | ura3/ura3 | | |
| YU75 | 3′ URA3 -P.ETF1-CbADC1-T.POX4 + | YU1 | pMW55 + |
| | P.TDH3-CbADH1-T.ETF1- 5′ URA3 3′ | | pMW58 |
| | URA3 − P.ETF1-TbACT1-T.POX4 - 5′ | | |
| | URA3 ura3/ura3 | | |
| YU76 | 3′ URA3 -P.ETF1-CbADC1-T.POX4 + | YU1 | pMW55 + |
| | P.TDH3-CbADH1-T.ETF1- 5′ URA3 3′ | | pMW58 |
| | URA3 − P.ETF1-TbACT1-T.POX4 - 5′ | | |
| | URA3 ura3/ura3 | | |
| YU77 | 3′ URA3 -P.ETF1-CbADC1-T.POX4 + | YU1 | pMW55 + |
| | P.TDH3-CbADH1-T.ETF1- 5′ URA3 3′ | | pMW58 |
| | URA3 − P.ETF1-TbACT1-T.POX4 - 5′ | | |
| | URA3 ura3/ura3 | | |

### Fermentation of YU3, YU72, YU73, YU74, YU75, YU76, and YU77

Three overnight of YU3, YU72, YU73, YU74, YU75, YU76, and YU77 in 3 ml of ScD-ura media were started from a single colony from freshly streaked ScD-ura plate and incubated at 30° ° C. overnight. 3 ml was used to inoculate 30 ml of YPD in a 250 ml flask and shake for 24 hours at 30° C.

Cultures were spun down, and the cells resuspended in 15 ml of SmP media with 6% dextrose. The culture was then transferred to a deep baffled 250 ml Erlenmeyer flask and incubated for two days at 30° C. Samples were taken, the cells were centrifuged, and the supernatant was filtered through a 0.22 um PTFE filter. The concentration of isopropanol and acetone was determined.

### Fermentation of YU3, YU72, YU73, and YU74

Three overnight of YU3, YU72, YU73, and Y74 in 3 ml of ScD-ura media was started from a single colony of freshly streaked ScD-ura plate and incubated at 30° C. overnight. 3 ml was used to inoculate 30 ml of YPD in a 250 ml flask and shake for 24 hours at 30° C.

Cultures were spun down, and the cells resuspended in 15 ml of SmP media with 4% dextrose. The culture was then transferred to a deep baffled 250 ml Erlenmeyer flask and incubated for six hours at 30° C. After six hours, 74 μl of acetone was added to one set and 1.5 ml of a lithium acetate solution (50 g/L) to another. After one and 3 hours, samples were taken. The cells were centrifuged and the supernatant filtered through a 0.22 um PTFE filter. The concentration of isopropanol and acetone was determined. Results are shown in Table 13.

TABLE 13
Concentrations of acetone and isopropanol in fermented cultures.

| | | 1 hr | | 3 hr | |
|---|---|---|---|---|---|
| | Addition | Acetone | Isopropanol | Acetone | Isopropanol |
| YU3 | — | 0 | 0 | 0 | 0 |
| YU72 | — | 0 | 0 | 0 | 0 |
| YU73 | — | 0 | 0 | 0 | 0 |
| YU74 | — | 0 | 0 | N/A | N/A |
| YU3 | Acetone | 0.387 | 0.033 | N/A | N/A |
| YU72 | Acetone | 0.266 | 0.188 | 0.154 | 0.224 |
| YU73 | Acetone | 0.322 | 0.155 | 0.248 | 0.27 |
| YU74 | Acetone | 0.214 | 0.11 | 0.183 | 0.159 |
| YU3 | Li-acetoacetate | 0.025 | 0.052 | 0.04 | 0 |
| YU72 | Li-acetoacetate | 0.02 | 0.084 | 0.033 | 0.066 |
| YU73 | Li-acetoacetate | 0.02 | 0.031 | 0.021 | 0.052 |
| YU74 | Li-acetoacetate | 0.012 | 0.028 | 0.023 | 0.042 |

No acetone or isopropanol was detected when no acetone or lithium acetoacetate was added. In contrast, an increase of isopropanol produced when acetone was added was observed in YU72, YU73, and YU74 versus the YU3 control. This result is consistent with the activity of the CbADH1 gene. No constant increase of acetone or isopropanol was observed when lithium acetoacetate was added.

### Construction of Strains YU58, YU59, YU61, YU62, and YU63

YU1 was transformed with Pac1 digested of either pMW38, pMW39, or pMW40 plasmids and plated in ScD-ura plate. URA+colonies were restreaked, and single colonies were used to start a 5 ml ScD-ura overnight at 30° C. Genomic DNA was extracted, and the integration was verified with the PCR method described above.

Characteristics of the resulting strains are shown in Table 14.

TABLE 14
Strains YU58, YU59, YU61, YU62, and YU63.

| | | Parental | |
|---|---|---|---|
| Strain | Genotype | Strain | Plasmid |
| YU58 | 3′ URA3-P.ETF1-CbADH1-T.POX4-5′ | YU1 | pMW38 |
| | URA3 ura3/ura3 | | |
| YU59 | 3′ URA3-P.ETF1-CbADH1-T.POX4-5′ | YU1 | pMW38 |
| | URA3 ura3/ura3 | | |
| YU60 | 3′ URA3-P.ETF1-NcADH1-T.POX4-5′ | YU1 | pMW39 |
| | URA3 ura3/ura3 | | |
| YU61 | 3′ URA3-P.ETF1-NcADH1-T.POX4-5′ | YU1 | pMW39 |
| | URA3 ura3/ura3 | | |
| YU62 | 3′ URA3-P.ETF1-CpADH1-T.POX4-5′ | YU1 | pMW40 |
| | URA3 ura3/ura3 | | |
| YU63 | 3′ URA3-P.ETF1-CpADH1-T.POX4-5′ | YU1 | pMW40 |
| | URA3 ura3/ura3 | | |

### Fermentation of YU3, YU58, YU59, YU61, YU62 and YU63.

Overnight of YU3, YU58, YU59, YU61, YU62, and YU63 in 3 ml of ScD-ura media was started from a single colony of freshly streaked ScD-ura plate and incubated at 30° C. overnight. 3 ml was used to inoculate 30 ml of YPD in a 250 ml flask and shake for 24 hours at 30° C. Cells were spun down, washed with 25 ml of water, and resuspended in 15 ml of SmP. The cells were transferred to a deep-baffled 250 ml shake flask, and 670 μl of oleic acid and 100 μl of a 50% glycerol solution were added. After six hours of incubation, 75 μl of isopropanol or acetone was added. Samples were taken at 1 and 3 hours, and the concentration of acetone and isopropanol was determined by GC. Results are shown in Table 15.

TABLE 15
Concentrations of acetone and isopropanol in fermented cultures.

| | Acetone --> Isopropanol | | | | Isopropanol --> Acetone | | | |
|---|---|---|---|---|---|---|---|---|
| | 1 hr | | 3 hr | | 1 hr | | 3 hr | |
| | Acetone | Isopropanol | Acetone | Isopropanol | Isopropanol | Acetone | Isopropanol | Acetone |
| YU3 | 0.290 | 0.010 | 0.294 | 0.019 | 0.145 | 0.151 | 0.028 | 0.238 |
| YU58 | 0.141 | 0.051 | 0.217 | 0.073 | 0.227 | 0.121 | 0.135 | 0.218 |
| YU59 | 0.180 | 0.176 | 0.123 | 0.241 | 0.252 | 0.075 | 0.190 | 0.107 |
| YU61 | 0.287 | 0.017 | 0.286 | 0.022 | 0.175 | 0.154 | 0.026 | 0.261 |
| YU62 | 0.198 | 0.016 | 0.272 | 0.031 | 0.172 | 0.156 | 0.014 | 0.231 |
| YU63 | 0.301 | 0.026 | 0.245 | 0.019 | 0.170 | 0.150 | 0.035 | 0.268 |

YU58 and YU59 produced more isopropanol from acetone than any other strain. That is consistent with activity from CbADH1. In contrast, we saw high production of acetone from isopropanol in all the strains except YU58 and YU59. This result is consistent with an elevated endogenous secondary alcohol dehydrogenase. The most likely alcohol dehydrogenase is CvADH5, which has a high identity to the CpADH1, a well-characterized secondary alcohol. CpADH1 and NcADHI utilize NADH as a cofactor to reduce acetone to isopropanol.

In contrast, CbADH1 used NADPH. These results are consistent with a high level of NADPH and a low level of NADH. When CbADH1 is present, it pushes the conversion of acetone to isopropanol. In the absence of it, the endogenous activity pushes the inverse conversion, isopropanol to acetone.

### Construction of Additional pVM Plasmids

pVM plasmids in Table 16 are synthesized by Twist Biosciences (USA).

TABLE 16
Additional pVM plasmids constructed.

| Plasmid | 5′ Addition | Sequence | 3′ Addition | Background |
|---|---|---|---|---|
| pVM88 | ACAGCTCTTCC | HiybgC | TAAGAGGGAAGAGC | pTwist-Kan- |
| | (SEQ ID NO: 137) | (SEQ ID NO: 51) | TGTACA | High-Copy |
| | | | (SEQ ID NO: 141) | |
| pVM89 | ACAGCTCTTCC | RgybgC | TAAGAGGGAAGAGC | pTwist-Kan- |
| | (SEQ ID NO: 137) | (SEQ ID NO: 56) | TGTACA | High-Copy |
| | | | (SEQ ID NO: 141) | |
| pVM90 | ACAGCTCTTCC | Hhybgc | TAAGAGGGAAGAGC | pTwist-Kan- |
| | (SEQ ID NO: 137) | (SEQ ID NO: 52) | TGTACA | High-Copy |
| | | | (SEQ ID NO: 141) | |
| pVM92 | ACAGCTCTTCC | CdADC1 | TAAGAGGGAAGAGC | pTwist-Kan- |
| | (SEQ ID NO: 137) | (SEQ ID NO: 101) | TGTACA | High-Copy |
| | | | (SEQ ID NO: 141) | |
| pVM93 | ACAGCTCTTCC | CsADC1 | TAAGAGGGAAGAGC | pTwist-Kan- |
| | (SEQ ID NO: 137) | (SEQ ID NO: 102) | TGTACA | High-Copy |
| | | | (SEQ ID NO: 141) | |
| pVM94 | ACAGCTCTTCC | ADH5 | TAAGAGGGAAGAGC | pTwist-Kan- |
| | (SEQ ID NO: 137) | (SEQ ID NO: 116) | TGTACA | High-Copy |
| | | | (SEQ ID NO: 141) | |
| pVM95 | | 5′ ADH5a | | pTwist- |
| | | (SEQ ID NO: 133) | | Chlor-High- |
| | | | | Copy |
| pVM96 | | 3′ADH5a | | pTwist-Kan- |
| | | (SEQ ID NO: 134) | | High-Copy |
| pVM97 | | 5′ ADH5b | | pTwist- |
| | | (SEQ ID NO: 135) | | Chlor-High- |
| | | | | Copy |
| pVM98 | | 3′ ADH5b | | pTwist-Kan- |
| | | (SEQ ID NO: 136) | | High-Copy |
| pVM99 | ACAGCTCTTCC | CsCTF1 | TAAGAGGGAAGAGC | pTwist-Kan- |
| | (SEQ ID NO: 137) | (SEQ ID NO: 31) | TGTACA | High-Copy |
| | | | (SEQ ID NO: 141) | |
| pVM100 | ACAGCTCTTCC | CsCTF2 | TAAGAGGGAAGAGC | pTwist-Kan- |
| | (SEQ ID NO: 137) | (SEQ ID NO: 32) | TGTACA | High-Copy |
| | | | (SEQ ID NO: 141) | |
| pVM101 | ACAGCTCTTCC | CaCTF1 | TAAGAGGGAAGAGC | pTwist-Kan- |
| | (SEQ ID NO: 137) | (SEQ ID NO: 33) | TGTACA | High-Copy |
| | | | (SEQ ID NO: 141) | |
| pVM102 | ACAGCTCTTCC | CaCTF2 | TAAGAGGGAAGAGC | pTwist-Kan- |
| | (SEQ ID NO: 137) | (SEQ ID NO: 34) | TGTACA | High-Copy |
| | | | (SEQ ID NO: 141) | |
| pVM103 | ACAGCTCTTCC | CtCTF1 | TAAGAGGGAAGAGC | pTwist-Kan- |
| | (SEQ ID NO: 137) | (SEQ ID NO: 35) | TGTACA | High-Copy |
| | | | (SEQ ID NO: 141) | |
| pVM104 | ACAGCTCTTCC | CtCTF2 | TAAGAGGGAAGAGC | pTwist-Kan- |
| | (SEQ ID NO: 137) | (SEQ ID NO: 36) | TGTACA | High-Copy |
| | | | (SEQ ID NO: 141) | |
| pVM105 | ACAGCTCTTCC | EcCTF1 | TAAGAGGGAAGAGC | pTwist-Kan- |
| | (SEQ ID NO: 137) | (SEQ ID NO: 37) | TGTACA | High-Copy |
| | | | (SEQ ID NO: 141) | |
| pVM106 | ACAGCTCTTCC | EcCTF2 | TAAGAGGGAAGAGC | pTwist-Kan- |
| | (SEQ ID NO: 137) | (SEQ ID NO: 38) | TGTACA | High-Copy |
| | | | (SEQ ID NO: 141) | |
| pVM107 | ACAGCTCTTCC | HpybgC | TAAGAGGGAAGAGC | pTwist-Kan- |
| | (SEQ ID NO: 137) | (SEQ ID NO: 53) | TGTACA | High-Copy |
| | | | (SEQ ID NO: 141) | |
| pVM108 | ACAGCTCTTCC | RtybgC | TAAGAGGGAAGAGC | pTwist-Kan- |
| | (SEQ ID NO: 137) | (SEQ ID NO: 54) | TGTACA | High-Copy |
| | | | (SEQ ID NO: 141) | |
| pVM109 | ACAGCTCTTCC | RmybgC | TAAGAGGGAAGAGC | pTwist-Kan- |
| | (SEQ ID NO: 137) | (SEQ ID NO: 55) | TGTACA | High-Copy |
| | | | (SEQ ID NO: 141) | |
| pVM111 | ACAGCTCTTCC | BxSrfAD | TAAGAGGGAAGAGC | pTwist-Kan- |
| | (SEQ ID NO: 137) | (SEQ ID NO: 57) | TGTACA | High-Copy |
| | | | (SEQ ID NO: 141) | |
| pVM112 | ACAGCTCTTCC | BaSrfAD | TAAGAGGGAAGAGC | pTwist-Kan- |
| | (SEQ ID NO: 137) | (SEQ ID NO: 58) | TGTACA | High-Copy |
| | | | (SEQ ID NO: 141) | |
| pVM113 | ACAGCTCTTCC | BhSrfAD | TAAGAGGGAAGAGC | pTwist-Kan- |
| | (SEQ ID NO: 137) | (SEQ ID NO: 59) | TGTACA | High-Copy |
| | | | (SEQ ID NO: 141) | |
| pVM114 | ACAGCTCTTCC | SmACL | TAAGAGGGAAGAGC | pTwist-Kan- |
| | (SEQ ID NO: 137) | (SEQ ID NO: 60) | TGTACA | High-Copy |
| | | | (SEQ ID NO: 141) | |
| pVM115 | ACAGCTCTTCC | ExACL | TAAGAGGGAAGAGC | pTwist-Kan- |
| | (SEQ ID NO: 137) | (SEQ ID NO: 61) | TGTACA | High-Copy |
| | | | (SEQ ID NO: 141) | |
| pVM116 | ACAGCTCTTCC | SfACL | TAAGAGGGAAGAGC | pTwist-Kan- |
| | (SEQ ID NO: 137) | (SEQ ID NO: 62) | TGTACA | High-Copy |
| | | | (SEQ ID NO: 141) | |
| pVM117 | ACAGCTCTTCC | CcADC1 | TAAGAGGGAAGAGC | pTwist-Kan- |
| | (SEQ ID NO: 137) | (SEQ ID NO: 93) | TGTACA | High-Copy |
| | | | (SEQ ID NO: 141) | |
| pVM118 | ACAGCTCTTCC | CpADC1 | TAAGAGGGAAGAGC | pTwist-Kan- |
| | (SEQ ID NO: 137) | (SEQ ID NO: 94) | TGTACA | High-Copy |
| | | | (SEQ ID NO: 141) | |
| pVM119 | ACAGCTCTTCC | PmADC1 | TAAGAGGGAAGAGC | pTwist-Kan- |
| | (SEQ ID NO: 137) | (SEQ ID NO: 95) | TGTACA | High-Copy |
| | | | (SEQ ID NO: 141) | |
| pVM120 | ACAGCTCTTCC | BaADC1 | TAAGAGGGAAGAGC | pTwist-Kan- |
| | (SEQ ID NO: 137) | (SEQ ID NO: 96) | TGTACA | High-Copy |
| | | | (SEQ ID NO: 141) | |
| pVM121 | ACAGCTCTTCC | PfADC1 | TAAGAGGGAAGAGC | pTwist-Kan- |
| | (SEQ ID NO: 137) | (SEQ ID NO: 97) | TGTACA | High-Copy |
| | | | (SEQ ID NO: 141) | |
| pVM122 | ACAGCTCTTCC | CeADC1 | TAAGAGGGAAGAGC | pTwist-Kan- |
| | (SEQ ID NO: 137) | (SEQ ID NO: 98) | TGTACA | High-Copy |
| | | | (SEQ ID NO: 141) | |
| pVM123 | ACAGCTCTTCC | BxADC1 | TAAGAGGGAAGAGC | pTwist-Kan- |
| | (SEQ ID NO: 137) | (SEQ ID NO: 100) | TGTACA | High-Copy |
| | | | (SEQ ID NO: 141) | |

### Construction of pFP Plasmids

Cloning is done using a GoldenBraid inspired method (Sarrion-Perdigones et al. 2013). In a 125 μl PCR tube, 5 ng of the receipt vector and 3× times equimolar of the one, two plasmids, two oligos, or PCR fragment that contain the inserts is combined. One μl of T4 DNA Ligase Buffer (New England Biolabs, USA), 0.5 μl of T4 DNA Ligase (New England Biolabs, USA), 0.5 μl of Bsal or SapI enzyme (New England Biolabs, USA), and water is added to a final volume of 10 μl. The reaction is then placed in a thermocycler and subjected to the following program:
1 cycle at 37° C. for 30 see
25 cycles of 37° ° C. for 2 min followed by 16° C. for 5 minutes
1 cycle of 37° ° C. for 10 min
1 cycle of 80° ° C. for 5 min

One μl of the reaction is then transformed into DH5alpha cells (Monserate Biotechnology Group, USA) as recommended by the manufacturing and plated in selective media. The final construct is verified by restriction enzyme analysis. See Table 17 for specific inserts used for each pFP plasmid.

The open reading frame of CvERG10 from pVM13 to make pFP4 is amplified with oligos o1 and o3 using Q5 High-Fidelity 2X Master Mix (New England Biolabs, USA) as recommended by the manufacturer. The PCR fragment is purified using DNA Clean & Concentrator-5, Capped Columns, as recommended by the Manufacturer (Zymo Research, USA). The concentration is determined using the DS DNA Broad Range kit by Denovix (USA) as recommended.

The PCR product is used as an insert in a GoldenBraid ligation as described above with the other parts shown in the table below to make pFP4. The final construct is verified by restriction enzyme analysis, and the sequence verified of the amplified open reading frame.

TABLE 17
Specific inserts for constructing pFP plasmids.

| Plasmid | Recipient | Insert 1 | Insert 2 | Enzyme | Selection |
|---|---|---|---|---|---|
| pFP1 | pVM95 | pMW1 | pMW96 | SapI | Kanamycin |
| pFP2 | pVM95 | pMW1 | pMW96 | SapI | Kanamycin |
| pFP3 | pVM20 | pVM13 | pVM21 | SapI | Ampicillin |
| pFP4 | pVM20 | PCR o1- | pVM21 | SapI | Ampicillin |
| | | o36(pVM13) | | | |
| pFP5 | pFP1 | pMW19 | pFP3 | BsaI | Chloramphenicol |
| pFP6 | pFP2 | pMW19 | pFP3 | BsaI | Chloramphenicol |
| pFP7 | pFP1 | pMW20 | pFP4 | BsaI | Chloramphenicol |
| pFP8 | pFP2 | pMW20 | pFP4 | BsaI | Chloramphenicol |
| pFP9 | pMW63 | pMW19 | pFP3 | BsaI | Chloramphenicol |
| pFP10 | pMW64 | pMW19 | pFP3 | BsaI | Chloramphenicol |

### Construction of Adh5 Deletion Strain

ATCC20913 (American Type Culture Collection, USA) is transformed with a PacI digested pFP1 and plated in ScD-ura plate. URA+colonies are restreaked, and single colonies are used to start a 5 ml YPD overnight at 30° C. Genomic DNA is extracted, and verification of deletion of the first allele of ADH5 is verified by PCR using Apex Taq RED Master Mix (Genesee Scientific) as recommended and using oligos of1 and o11, and oligos of2 and o10. A strain with the correct amplification bands is named FY1.

100 μl of the overnight of FY1 is plated in ScD+5-FOA plates and is incubated at 30° C. for a couple of days. 5-FOA resistant colonies are restreaked in ScD+5-FOA plates, and single colonies are used to start a 5 ml YPD overnight. Genomic DNA is extracted, and the loop out of the URA3 in the ADH5 loci is verified by PCR using Apex Taq RED Master Mix (Genesee Scientific) as recommended and using oligos of1 and of2. A strain with the correct amplification bands is named FY2.

FY2 is transformed with a PacI digested pFP2 and plated in ScD-ura plate. URA+colonies are restreaked, and single colonies are used to start a 5 ml YPD overnight at 30° C. Genomic DNA is extracted, and the deletion of the second allele of ADH5 is verified by PCR using Apex Taq RED Master Mix (Genesee Scientific) as recommended and using oligos o12, o13, of4, and of5. Oligos o12 and o13 amplify an 803 bp nucleotide fragment of the actin gene, while of4 and of5 amplify a 366 nucleotide fragment of the ADH5 gene. A strain with the correct amplification bands is named FY3.

100 μl of the overnight of FY3 is plated in ScD+5-FOA plates and incubated at 30 ºC for a couple of days. 5-FOA resistant colonies are restreaked in ScD+5-FOA plates, and single colonies are used to start a 5 ml YPD overnight. Genomic DNA is extracted, and the loop out of the URA3 in the ADH5 loci is verified by PCR using Apex Taq RED Master Mix (Genesee Scientific) as recommended and using oligos o1 and o3, and oligos o1 and o2. A strain with the correct amplification bands is named FY4.

This strain is then used to test different secondary alcohol dehydrogenase. If the endogenous activity is detrimental, this strain then becomes the base strain for all genetic manipulations.

### Construction of Strain Overexpressing CvERG10

ATCC20913 (American Type Culture Collection, USA) is transformed with a PacI digested pFP5 and plated in ScD-ura plate. URA+colonies are restreaked, and single colonies are used to start a 5 ml YPD overnight at 30° C. Genomic DNA is extracted, and deletion of the first allele of ADH5 is verified by PCR using Apex Taq RED Master Mix (Genesee Scientific) as recommended and using oligos that bind outside to the area complementary to the flanking regions and the inserted DNA piece. A strain with the correct amplification bands is used for the next step.

100 μl of the overnight of the correct strain is plated in ScD+5-FOA plates and is incubated at 30° C. for a couple of days. 5-FOA resistant colonies are restreaked in ScD+5-FOA plates, and single colonies are used to start a 5 ml YPD overnight. Genomic DNA is extracted, and the loop out of the URA3 in the ADH5 loci is verified by PCR using Apex Taq RED Master Mix (Genesee Scientific). A strain with the correct amplification bands is used for the next step.

The correct strain is transformed with a PacI digested pFP6 and plated in ScD-ura plate. URA+colonies are restreaked, and single colonies are used to start a 5 ml YPD overnight at 30° C. Genomic DNA is extracted, and the deletion of the second allele of ADH5 is verified by PCR using Apex Taq RED Master Mix (Genesee Scientific) as recommended and using oligos o12, o13, o4, and o5. Oligos o12 and o13 amplifies a 803 nucleotide fragment of the actin gene, while o4 and o5 amplify a 366 nucleotide fragment of the ADH5 gene. A strain with the correct amplification bands is used for the next step.

If the URA3 is needed for another genetic manipulation, 100 μl of the overnight of correct strain is plated in ScD+5-FOA plates and incubated at 30° C. for a couple of days. 5-FOA resistant colonies are restreaked in ScD+5-FOA plates, and single colonies are used to start a 5 ml YPD overnight. Genomic DNA is extracted, and loop out of the URA3 in the ADH5 loci is verified by PCR using Apex Taq RED Master Mix (Genesee Scientific) as recommended. A strain with the correct amplification bands is used for the next step.

More copies could be inserted in other regions of the genome in addition to the ADH5 loci.

Insertion of Two Copies of CvERG10 into the GRE3 Loci.

A similar method is used as the previous example, except pFP7 and pFP8 are used instead of pFP5 and pFP6, respectively.

### Expression of Other ERG10 Genes.

Other ERG10 genes may also increase acetone and isopropanol production yield from fatty acids. Similar examples to the one described are repeated, except the open reading of CvERG10 is replaced with the open reading frame of CjERG10 from pVM67.

### Expression of Acetoacetyl-CoA Transferase Enzymes

In addition to the acetoacetyl-CoA transferase from Clostridium, other acetoacetyl-CoA transferases can be used to catalyze the acetoacetyl-CoA to acetoacetate step. These genes include genes from Clostridium saccharobutylicum, Clostridium algidicarnis, Clostridium thermoalcaliphilum, and Escherichia coli.

Expression of these proteins are done is a similar manner as expression of CbCTF1 and CbCTF2. The CbCTF1 and CbCTF2 open reading frames are replaced with the open reading frames of CsCTF1 and CsCTF2, CaCTF1 and CaCTF2, CtCTF1 and CtCTF2, or EcCTF1 and EcCTF2 found in plasmids pVM99, pVM100, pVM101, pVM102, pVM103, pVM104, pVM105, and pVM106, respectively (Table 16). The effect of the expression is then determined by fermentation as described before.

### Expression of Thioesterases Enzymes

Another option to catalyze acetoacetyl-CoA to acetoacetate step is to use a thioesterase. Some examples with potential thioesterase activity are HiybgC, HhybgC, HpybgC, RtybgC, RmybgC, RgYBGC, BxSrfAD, BaSrfAD, BhSrfAD, SmACL, ExACL, and SfACL from Haemophilus influenzae, Haemophilus haemolyticus, Haemophilus parainfluenzae, Rodentibacter trehalosifermentans, Rodentibacter myodis, Rodentibacter genomosp. 2, Bacillus, Bacillus atrophaeus, Bacillus halotolerans, Sinorhizobium meliloti, Ensifer, or Sinorhizobium fredii, respectively.

Expression of these proteins are done a similar manner as the expression of CbCTF1 and CbCTF2, except you replace CbCTF1 with the open reading frame of the thioesterases, and you do not use CbCTF2. You replace the open reading frame of CbCTF1 with HiybgC, HhybgC, HpybgC, RtybgC, RmybgC, RgYBGC, BxSrfAD, BaSrfAD, BhSrfAD, SmACL, ExACL, or SfACL found in plasmids pVM88, pVM90, pVM107, pVm108, pVM109, pVM89, pVM111, pVM112, pVM113, PVM114, pVM115, and pVM116, respectively (Table 16). The effect of the expression is then determined by fermentation, as described before.

### Expression of Acetoacetate Decarboxylase

In addition to the acetoacetate decarboxylase from Clostridium, other acetoacetate decarboxylases can be used to catalyze the acetoacetate to acetone step. This includes genes from Clostridium cagae, Clostridium pasteurianum, Priestia megaterium, Bacillus acidicola, Pelosinus fermentans, Clostridium estertheticum, Brevibacterium, Clostridium sp DL-VII and Clostridium saccharoperbutylacetonicum.

Expression of these proteins is done similarly to the expression of CbADC1. You replace the open reading frame of CbADC1 with CdADC1, CsADC1, CcADC1, CpADC1, PmADC1, BaADC1, PfADC1, CeADC1, or BxADC1 found in plasmids pVM92, pVM93, pVM117, pVM118, pVM119, pVM120, pVM121, pVM122, or pVM123, respectively (Table 16). The effect of the expression is then determined by fermentation, as described before.

### Other Promoters

Other promoters can be used to drive the acetoacetate-CoA C-transferase, thioesterase, acetoacetate decarboxylase, or secondary alcohol dehydrogenase encoding genes. These promoters will have high-level expression; constitutive or fatty-acid inducible promoters. Promoters from genes encoding enzymes involved in central carbon metabolism, general transcription or general translation are a good candidate for high expression constitutive promoters. Promoters that encode genes responsible for the oxidation or degradation of fatty acids could be suitable inducible promoters. 500 to 1000 bp upstream from the starting codon of the protein are cloned in fusion with the open reading frame to be expressed similarly as the ETF1, POX4, or PEX11 promoter were fused.

### Localization of Enzymes to the Peroxisome.

There may be a benefit from localizing all or some of the enzymes of the isopropanol pathway in the peroxisome. Enzymes are targeted to the peroxisome by the addition of a peroxisomal targeting signal (PTS). An additional eighteen base pairs encoding GRRAKL are added to the open reading frame at the carboxyl-terminus of the protein. The peroxisomal targeted open reading frame replaces the non-peroxisomal targeted open reading frame to the cloning described above, and they get integrated into the genome as described. The effect of the expression is then determined by fermentation, as described before. See Table 18 for different options for the localization of the enzymes.

TABLE 18
Options for localization of the enzymes in the peroxisome.

| | Activity | | | |
|---|---|---|---|---|
| | | Acetoacetyl-CoA | | Secondary |
| | Acetyl-CoA | transferase | | Alcohol |
| | C-acetyl- | (E3 activity)/ | Acetoacetate | Dehydro- |
| | transferase | Thioesterase | decarboxylase | genase |
| | (E2 activity) | (E4 activity) | (E5 activity) | (E6 activity) |
| FIGs. 9, | Cytoplasmic | Cytoplasmic | Cytoplasmic | Cytoplasmic |
| 10, 14, | | | | |
| and 15 | | | | |
| FIG. 16 | Peroxisomal | Peroxisomal | Cytoplasmic | Cytoplasmic |
| FIG. 17 | Peroxisomal | Peroxisomal | Peroxisomal | Cytoplasmic |
| FIG. 18 | Peroxisomal | Peroxisomal | Cytoplasmic | Peroxisomal |

### Expression of an Acetyl-CoA Thioesterase

Expression of acetyl-CoA thioesterase in addition to the acetoacetate-CoA C-transferase may improve the production of acetone or isopropanol by moving the acetate from the peroxisome or providing the acetate for an acetoacetyl-CoA transferase. The open reading frame encoding an enzyme such as the one encoded by TbACT1 is placed under the control of a constitutive or fatty-acid promoter as described for other genes. Then the gene is inserted either randomly or integrated into a specific genome site. The effect of the expression is then determined by fermentation, as described before.

### REFERENCES

Dellomonaco, C., Rivera, C., Campbell, P., & Gonzalez, R. (2010). Engineered Respiro-Fermentative Metabolism for the Production of Biofuels and Biochemicals from Fatty Acid-Rich Feedstocks. Applied and Environmental Microbiology, 76(15), 5067-5078.
Tamakawa, H., Mita, T., Yokoyama, A., Ikushima, S., & Yoshida, S. (2013). Metabolic engineering of Candida utilis for isopropanol production. Applied Microbiology and Biotechnology, 97(14), 6231-6239.

1. A recombinant yeast comprising one or more recombinant genes, wherein the one or more recombinant genes are configured to express any one or more enzymes selected from the group consisting of an acetyl-CoA thioesterase, an acetyl-CoA C-acetyltransferase, an acetoacetyl-CoA transferase, an acetoacetyl-CoA thioesterase, an acetoacetate decarboxylase, and an isopropanol dehydrogenase.

2. The recombinant yeast of claim 1, wherein the one or more recombinant genes are configured to express any one or more enzymes selected from the group consisting of an acetyl-CoA thioesterase comprising a sequence at least 90% identical to any one of SEQ ID NOs:1-3; an acetyl-CoA C-acetyltransferase comprising a sequence at least 90% identical to any one of SEQ ID NOs:7-10; an acetoacetyl-CoA transferase comprising a sequence at least 90% identical to any one of SEQ ID NOs:15-26; an acetoacetyl-CoA thioesterase comprising a sequence at least 90% identical to any one of SEQ ID NOs:1-3 and 39-50; an acetoacetate decarboxylase comprising a sequence at least 90% identical to any one of SEQ ID NOs:63-82; and an isopropanol dehydrogenase comprising a sequence at least 90% identical to any one of SEQ ID NOs: 103-109.

3. The recombinant yeast of claim 1, wherein the one or more recombinant genes are configured to express any one or more enzymes selected from the group consisting of the acetoacetyl-CoA transferase, the acetoacetate decarboxylase, and the isopropanol dehydrogenase.

4. The recombinant yeast of claim 1, wherein the one or more recombinant genes are configured to express the acetoacetyl-CoA transferase.

5. The recombinant yeast of claim 1, wherein the acetoacetyl-CoA transferase comprises a sequence at least 90% identical to any one of SEQ ID NOs: 15 and 17.

6. The recombinant yeast of claim 1, wherein the one or more recombinant genes are configured to express the acetoacetate decarboxylase.

7. The recombinant yeast of claim 1, wherein the acetoacetate decarboxylase comprises a sequence at least 90% identical to SEQ ID NO:63.

8. The recombinant yeast of claim 1, wherein the one or more recombinant genes are configured to express the isopropanol dehydrogenase.

9. The recombinant yeast of claim 1, wherein the isopropanol dehydrogenase comprises a sequence at least 90% identical to SEQ ID NO:103.

10. The recombinant yeast of claim 1, wherein at least one of the one or more recombinant genes comprises a heterologous promoter.

11. The recombinant yeast of claim 1, wherein each of the one or more recombinant genes comprises a heterologous promoter.

12. The recombinant yeast of claim 1, wherein at least one of the one or more recombinant genes comprises a fatty acid inducible promoter.

13. The recombinant yeast of claim 1, wherein one of the one or more recombinant genes is configured to express the acetyl-CoA thioesterase and comprises a fatty acid inducible promoter.

14. The recombinant yeast of claim 1, wherein one of the one or more recombinant genes is configured to express the acetyl-CoA C-acetyltransferase and comprises a fatty acid inducible promoter.

15. The recombinant yeast of claim 1, wherein one of the one or more recombinant genes is configured to express the acetoacetyl-CoA transferase and comprises a fatty acid inducible promoter.

16. The recombinant yeast of claim 1, wherein one of the one or more recombinant genes is configured to express the acetoacetyl-CoA thioesterase and comprises a fatty acid inducible promoter.

17. The recombinant yeast of claim 1, wherein one of the one or more recombinant genes is configured to express the acetoacetate decarboxylase and comprises a fatty acid inducible promoter.

18. The recombinant yeast of claim 1, wherein one of the one or more recombinant genes is configured to express the isopropanol dehydrogenase and comprises a fatty acid inducible promoter.

19. The recombinant yeast of claim 1, wherein each fatty acid inducible promoter is independently selected from POX1 (Acyl-CoA Oxidase 1) promoter, POX2 (Acyl-CoA Oxidase 2) promoter, POX4 (Acyl-CoA Oxidase 4) promoter, POX5 (Acyl-CoA Oxidase 5) promoter, G3P (Glycerol-3-Phosphate Dehydrogenase) promoter, ICL1 (Isocitrate Lyase) promoter, POT1 (3-Oxo-Acyl-CoA Thiolase) promoter, LIP2 (Lipoyl ligase) promoter, TDH1 (Glyceraldehyde-3-Phosphate Dehydrogenase, isozyme 1) promoter, TDH3 (Glyceraldehyde-3-Phosphate Dehydrogenase, isozyme 3) promoter, PEX11 (Peroxisome Membrane Protein) promoter, or a variant of any of the foregoing.

20. The recombinant yeast of claim 1, wherein each fatty acid inducible promoter independently comprises a sequence at least 95% identical to any one of SEQ ID NOs: 117, 118, and 120.

21. The recombinant yeast of claim 1, wherein the yeast is a species of Candida.

22. The recombinant yeast of claim 1, wherein the yeast is Candida viswanathii.

23. The recombinant yeast of claim 1, wherein the yeast is Candida viswanathii and wherein the one or more recombinant genes comprise:
a recombinant gene configured to express the acetoacetyl-CoA transferase and comprising a fatty acid inducible promoter, wherein the acetoacetyl-CoA transferase comprises a sequence at least 95% identical to any one of SEQ ID NOs: 15 and 17, wherein the fatty acid inducible promoter comprises a sequence at least 95% identical to any one of SEQ ID NOs:117, 118, and 120; and
a recombinant gene configured to express the acetoacetate decarboxylase and comprising a fatty acid inducible promoter, wherein the acetoacetate decarboxylase comprises a sequence at least 95% identical to SEQ ID NO:63, wherein the fatty acid inducible promoter comprises a sequence at least 95% identical to any one of SEQ ID NOs:117, 118, and 120.

24. The recombinant yeast of claim 23, wherein the one or more recombinant genes comprise a recombinant gene configured to express the isopropanol dehydrogenase, wherein the isopropanol dehydrogenase comprises a sequence at least 95% identical to SEQ ID NO:103.

25. A method of producing a product, the method comprising culturing the yeast of claim 1 in a culture medium comprising fatty acids for a time sufficient to produce the product, wherein the product comprises one or more of acetone and isopropanol.

26. The method of claim 25, wherein the fatty acids are present in the culture medium in an amount of at least 1% v/v.

27. The method of claim 25, wherein the culture medium is devoid of fermentable sugar or contains less than 25% w/v fermentable sugar.
